SEQUENCE LISTING (11 sequences)

SEQ ID NO: 1 - protein, 450 aa, Artificial Sequence
  Heavy chain of humanized anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Light chain of humanized anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 8 aa, Artificial Sequence
  CDRH1 of heavy chain of humanized anti-HER2 antibody
GFNIKDTY

SEQ ID NO: 4 - protein, 8 aa, Artificial Sequence
  CDRH2 of heavy chain of humanized anti-HER2 antibody
IYPTNGYT

SEQ ID NO: 5 - protein, 13 aa, Artificial Sequence
  CDRH3 of heavy chain of humanized anti-HER2 antibody
SRWGGDGFYAMDY

SEQ ID NO: 6 - protein, 6 aa, Artificial Sequence
  CDRL1 of light chain of humanized anti-HER2 antibody
QDVNTA

SEQ ID NO: 7 - protein, 7 aa, Artificial Sequence
  Sequence comprising CDRL2 of light chain of humanized anti-HER2 antibody
SASFLYS

SEQ ID NO: 8 - protein, 9 aa, Artificial Sequence
  CDRL3 of light chain of humanized anti-HER2 antibody
QQHYTTPPT

SEQ ID NO: 9 - protein, 120 aa, Artificial Sequence [sequence omitted]

SEQ ID NO: 10 - protein, 107 aa, Artificial Sequence [sequence omitted]

SEQ ID NO: 11 - protein, 449 aa, Artificial Sequence
  Heavy chain of humanized anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2023/0256110 A1
(43) Pub. Date: Aug. 17, 2023

(54) COMBINATION OF ANTIBODY-DRUG CONJUGATE AND ATM INHIBITOR

(71) Applicants: AstraZeneca UK Limited, Cambridge (GB); DAIICHI SANKYO COMPANY, LIMITED, Tokyo (JP)

(72) Inventors: Jerome Thomas METTETAL, II, Wilmington,, DE (US); Stephen Thomas DURANT, Cambridge (GB); Azadeh Cheraghchi Bashi ASTANEH, Cambridge (GB); Alan Yin Kai LAU, Cambridge (GB); Yann WALLEZ, Cambridge (GB)

(73) Assignees: AstraZeneca UK Limited, Cambridge (GB); DAIICHI SANKYO COMPANY, LIMITED, Tokyo (JP)

(21) Appl. No.: 18/012,900

(22) PCT Filed: Jun. 23, 2021
(86) PCT No.: PCT/IB2021/055549

§ 371 Date: Dec. 23, 2022

Related U.S. Application Data
(60) Provisional application No. 63/043,474, filed on Jun. 24, 2020.

(51) Int. Cl. A61K 47/68; A61K 31/4745; C07K 16/22; A61P 35/00

(52) U.S. Cl. CPC A61K 47/6803; A61K 47/6855; A61K 47/6889; A61K 31/4745; C07K 16/22; A61P 35/00

(57) ABSTRACT

A pharmaceutical product for administration of an anti HER2 antibody-drug conjugate in combination with an ATM inhibitor is provided. The anti-HER2 antibody-drug conjugate is an antibody-drug conjugate in which a drug linker represented by the following formula (wherein A represents the connecting position to an antibody) is conjugated to an anti-HER2 antibody via a thioether bond. Also provided is a therapeutic use and method wherein the antibody-drug conjugate and the ATM inhibitor are administered in combination to a subject: Formula (I).

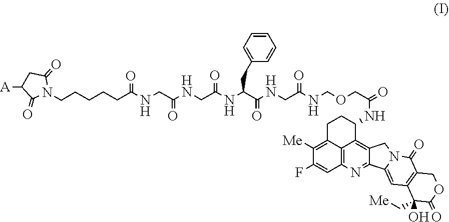

### TECHNICAL FIELD

The present disclosure relates to a pharmaceutical product for administration of a specific antibody-drug conjugate, having an antitumor drug conjugated to an anti-HER2 antibody via a linker structure, in combination with an ATM inhibitor, and to a therapeutic use and method wherein the specific antibody-drug conjugate and the ATM inhibitor are administered in combination to a subject.

### BACKGROUND

ATM (ataxia telangiectasia mutated kinase) is a serine/threonine protein kinase originally identified as the product of the gene mutated in ataxia telangiectasia. Ataxia telangiectasia is located on human chromosome 11q22-23 and codes for a large protein of about 350 kDa, which is characterized by the presence of a phosphatidylinositol (“PI”) 3-kinase-like serine/threonine kinase domain flanked by FRAP-ATM-TRRAP and FATC domains which modulate ATM kinase activity and function. ATM kinase has been identified as a major player of the DNA damage response elicited by double strand breaks. It primarily functions in S/G2/M cell cycle transitions and at collapsed replication forks to initiate cell cycle checkpoints, chromatin modification, HR repair and pro-survival signalling cascades in order to maintain cell integrity after DNA damage (Lavin, M. F.; Rev. Mol. Cell Biol. 2008, 759-769). ATM kinase responds to direct double strand breaks caused by common anti-cancer treatments such as ionising radiation and topoisomerase-II inhibitors (doxorubicin, etoposide) but also to topoisomerase-I inhibitors (for example irinotecan and topotecan) via single strand break to double strand break conversion during replication. ATM kinase inhibition can potentiate the activity of any these agents, and as a result ATM kinase inhibitors are expected to be of use in the treatment of cancer. Examples of ATM inhibitors are disclosed, for example, in WO2017/046216.

Antibody-drug conjugates (ADCs), which are composed of a cytotoxic drug conjugated to an antibody, can deliver the drug selectively to cancer cells, and are therefore expected to cause accumulation of the drug within cancer cells and to kill the cancer cells (Ducry, L., et al., Bioconjugate Chem. (2010) 21, 5-13; Alley, S. C., et al., Current Opinion in Chemical Biology (2010) 14, 529-537; Damle N. K. Expert Opin. Biol. Ther. (2004) 4, 1445-1452; Senter P. D., et al., Nature Biotechnology (2012) 30, 631-637; Burris H A., et al., J. Clin. Oncol. (2011) 29(4): 398-405).

One such antibody-drug conjugate is trastuzumab deruxtecan, which is composed of a HER2-targeting antibody and a derivative of exatecan (Ogitani Y. et al., Clinical Cancer Research (2016) 22(20), 5097-5108; Ogitani Y. et al., Cancer Science (2016) 107, 1039-1046).

Despite the therapeutic potential of antibody-drug conjugates and ATM inhibitors, no literature is published that describes a test result demonstrating an excellent effect of combined use of the antibody-drug conjugate and an ATM inhibitor or any scientific basis suggesting such a test result. Moreover, in the absence of test results, a possibility exists that combined administration of the antibody-drug conjugate together with another cancer treating agent such as an ATM inhibitor could lead to negative interactions and/or sub-additive therapeutic outcomes, and thus an excellent or superior effect obtained by such combination treatment could not be expected.

Accordingly, a need remains for improved therapeutic compositions and methods, that can enhance efficacy of existing cancer treating agents, increase durability of therapeutic response and/or reduce dose-dependent toxicity.

### SUMMARY OF DISCLOSURE

The antibody-drug conjugate used in the present disclosure (an anti-HER2 antibody-drug conjugate that includes a derivative of the topoisomerase I inhibitor exatecan) has been confirmed to exhibit an excellent antitumor effect in the treatment of certain cancers such as breast cancer and gastric cancer, when administered singly. However, it is desired to provide a medicine and treatment which can obtain a superior antitumor effect in the treatment of cancers, such as enhanced efficacy, increased durability of therapeutic response and/or reduced dose-dependent toxicity. By inhibiting the DNA damage response to double strand breaks introduced by the antibody-drug conjugate of the present disclosure, an ATM inhibitor may further enhance antitumor efficacy when administered in combination with the antibody-drug conjugate.

The present disclosure provides a pharmaceutical product which can exhibit an excellent antitumor effect in the treatment of cancers, through administration of an anti-HER2 antibody-drug conjugate in combination with an ATM inhibitor. The present disclosure also provides a therapeutic use and method wherein the anti-HER2 antibody-drug conjugate and ATM inhibitor are administered in combination to a subject.

Specifically, the present disclosure relates to the following [1] to [52]:

[1] a pharmaceutical product comprising an anti-HER2 antibody-drug conjugate and an ATM inhibitor for administration in combination, wherein the anti-HER2 antibody-drug conjugate is an antibody-drug conjugate in which a drug-linker represented by the following formula:

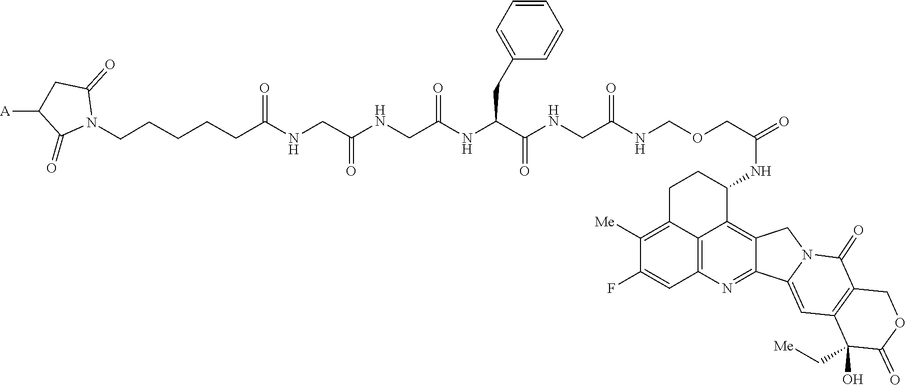

wherein A represents the connecting position to an antibody, is conjugated to an anti-HER2 antibody via a thioether bond;
[2] the pharmaceutical product according to [1], wherein the ATM inhibitor is a compound represented by the following formula (I):

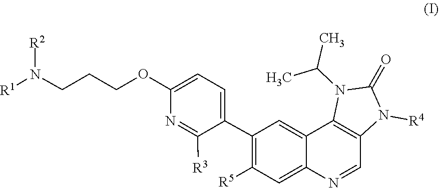

wherein:

R¹ is methyl;

R² is hydrogen or methyl; or R¹ and R² together with the nitrogen atom to which they are bonded form an azetidinyl, pyrrolidinyl or piperidinyl ring;

R³ is hydrogen or fluoro;

R⁴ is hydrogen or methyl; and

R⁵ is hydrogen or fluoro,

or a pharmaceutically acceptable salt thereof;
[3] the pharmaceutical product according to [2] wherein, in formula (I), R¹ and R² are both methyl; or R¹ and R² together with the nitrogen atom to which they are bonded form an azetidinyl, pyrrolidinyl or piperidinyl ring;
[4] the pharmaceutical product according to [2] or [3] wherein, in formula (I), R¹ and R² together with the nitrogen atom to which they are bonded form an azetidinyl, pyrrolidinyl or piperidinyl ring;
[5] the pharmaceutical product according to any one of [2] to [4] wherein, in formula (I), R³ is hydrogen;
[6] the pharmaceutical product according to any one of [2] to [5] wherein, in formula (I), R⁴ is methyl;
[7] the pharmaceutical product according to any one of [2] to [6] wherein the compound of formula (I) is R⁵ is fluoro;
[8] the pharmaceutical product according to [2] wherein, in formula (I):

R¹ is methyl;

R² is methyl; or R¹ and R² together with the nitrogen atom to which they are bonded form an azetidinyl, pyrrolidinyl or piperidinyl ring;

R³ is hydrogen or fluoro;

R⁴ is methyl; and

R⁵ is hydrogen or fluoro;

[9] the pharmaceutical product according to [2], wherein the ATM inhibitor is AZD1390, also known as AZ13791971, represented by the following formula:

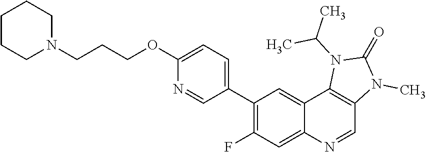 [SMILES: CC(C)n1c(=O)n(C)c2cnc3cc(F)c(-c4ccc(OCCCN5CCCCC5)nc4)cc3c21]

or a pharmaceutically acceptable salt thereof;
[10] the pharmaceutical product according to any one of [1] to [9], wherein the anti-HER2 antibody is an antibody comprising a heavy chain comprising CDRH1 consisting of an amino acid sequence represented by SEQ ID NO: 3 [=amino acid residues 26 to 33 of SEQ ID NO: 1], CDRH2 consisting of an amino acid sequence represented by SEQ ID NO: 4 [=amino acid residues 51 to 58 of SEQ ID NO: 1] and CDRH3 consisting of an amino acid sequence represented by SEQ ID NO: 5 [=amino acid residues 97 to 109 of SEQ ID NO: 1], and a light chain comprising CDRL1 consisting of an amino acid sequence represented by SEQ ID NO: 6 [=amino acid residues 27 to 32 of SEQ ID NO: 2], CDRL2 consisting of an amino acid sequence consisting of amino acid residues 1 to 3 of SEQ ID NO: 7 [=amino acid residues 50 to 52 of SEQ ID NO: 2] and CDRL3 consisting of an amino acid sequence represented by SEQ ID NO: 8 [=amino acid residues 89 to 97 of SEQ ID NO: 2];
[11] the pharmaceutical product according to any one of [1] to [9], wherein the anti-HER2 antibody is an antibody comprising a heavy chain comprising a heavy chain variable region consisting of an amino acid sequence represented by SEQ ID NO: 9 [=amino acid residues 1 to 120 of SEQ ID NO: 1] and a light chain comprising a light chain variable region consisting of an amino acid sequence represented by SEQ ID NO: 10 [=amino acid residues 1 to 107 of SEQ ID NO: 2];
[12] the pharmaceutical product according to any one of [1] to [9], wherein the anti-HER2 antibody is an antibody comprising a heavy chain consisting of an amino acid sequence represented by SEQ ID NO: 1 and a light chain consisting of an amino acid sequence represented by SEQ ID NO: 2;
[13] the pharmaceutical product according to any one of [1] to [9], wherein the anti-HER2 antibody is an antibody comprising a heavy chain consisting of an amino acid sequence represented by SEQ ID NO: 11 [=amino acid residues 1 to 449 of SEQ ID NO: 1] and a light chain consisting of an amino acid sequence represented by SEQ ID NO: 2;
[14] the pharmaceutical product according to any one of [1] to [13], wherein the anti-HER2 antibody-drug conjugate is represented by the following formula:

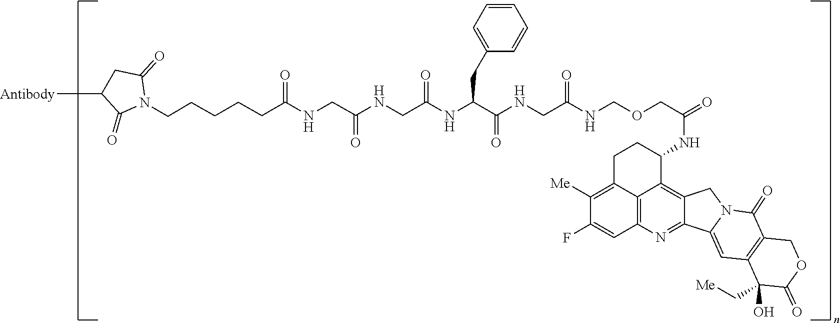

wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8;
[15] the pharmaceutical product according to any one of [1] to [14], wherein the anti-HER2 antibody-drug conjugate is trastuzumab deruxtecan (DS-8201);
[16] the pharmaceutical product according to any one of [1] to [15] wherein the product is a composition comprising the anti-HER2 antibody-drug conjugate and the ATM inhibitor, for simultaneous administration;
[17] the pharmaceutical product according to any one of [1] to [15] wherein the product is a combined preparation comprising the anti-HER2 antibody-drug conjugate and the ATM inhibitor, for sequential or simultaneous administration;
[18] the pharmaceutical product according to any one of [1] to [17], wherein the product is for treating cancer;
[19] the pharmaceutical product according to [18], wherein the cancer is at least one selected from the group consisting of breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioma, glioblastoma multiforme, osteosarcoma, sarcoma, and melanoma;
[20] the pharmaceutical product according to [18], wherein the cancer is breast cancer;
[21] the pharmaceutical product according to [20], wherein the breast cancer has a HER2 status score of IHC 3+;
[22] the pharmaceutical product according to [20], wherein the breast cancer is HER2 low-expressing breast cancer;
[23] the pharmaceutical product according to [20], wherein the breast cancer has a HER2 status score of IHC 2+;
[24] the pharmaceutical product according to [20], wherein the breast cancer has a HER2 status score of IHC 1+;
[25] the pharmaceutical product according to [20], wherein the breast cancer has a HER2 status score of IHC >0 and <1+;
[26] the pharmaceutical product according to [20], wherein the breast cancer is triple-negative breast cancer;
[27] the pharmaceutical product according to [18], wherein the cancer is gastric cancer;
[28] the pharmaceutical product according to [18], wherein the cancer is colorectal cancer;
[29] the pharmaceutical product according to [18], wherein the cancer is lung cancer;
[30] the pharmaceutical product according to [29], wherein the lung cancer is non-small cell lung cancer;
[31] the pharmaceutical product according to [18], wherein the cancer is pancreatic cancer;
[32] the pharmaceutical product according to [18], wherein the cancer is ovarian cancer;
[33] the pharmaceutical product according to [18], wherein the cancer is prostate cancer;
[34] the pharmaceutical product according to [18], wherein the cancer is kidney cancer;
[35] a pharmaceutical product as defined in any one of [1] to [17], for use in treating cancer;
[36] the pharmaceutical product for the use according to [35], wherein the cancer is as defined in any one of [19] to [34];
[37] use of an anti-HER2 antibody-drug conjugate or an ATM inhibitor in the manufacture of a medicament for administration of the anti-HER2 antibody-drug conjugate and the ATM inhibitor in combination, wherein the anti-HER2 antibody-drug conjugate and the ATM inhibitor are as defined in any one of [1] to [15], for treating cancer;
[38] the use according to [37], wherein the cancer is as defined in any one of [19] to [34];
[39] the use according to [37] or [38] wherein the medicament is a composition comprising the anti-HER2 antibody-drug conjugate and the ATM inhibitor, for simultaneous administration;
[40] the use according to [37] or [38] wherein the medicament is a combined preparation comprising the anti-HER2 antibody-drug conjugate and the ATM inhibitor, for sequential or simultaneous administration;
[41] an anti-HER2 antibody-drug conjugate for use, in combination with an ATM inhibitor, in the treatment of cancer, wherein the anti-HER2 antibody-drug conjugate and the ATM inhibitor are as defined in any one of [1] to [15];
[42] the anti-HER2 antibody-drug conjugate for the use according to [41], wherein the cancer is as defined in any one of [19] to [34];
[43] the anti-HER2 antibody-drug conjugate for the use according to [41] or [42], wherein the use comprises administration of the anti-HER2 antibody-drug conjugate and the ATM inhibitor sequentially;
[44] the anti-HER2 antibody-drug conjugate for the use according to [41] or [42], wherein the use comprises administration of the anti-HER2 antibody-drug conjugate and the ATM inhibitor simultaneously;
[45] an ATM inhibitor for use, in combination with an anti-HER2 antibody-drug conjugate, in the treatment of cancer, wherein the anti-HER2 antibody-drug conjugate and the ATM inhibitor are as defined in any one of [1] to [15];
[46] the ATM inhibitor for the use according to [45], wherein the cancer is as defined in any one of [19] to [34];
[47] the ATM inhibitor for the use according to [45] or [46], wherein the use comprises administration of the anti-HER2 antibody-drug conjugate and the ATM inhibitor sequentially;
[48] the ATM inhibitor for the use according to [45] or
[46], wherein the use comprises administration of the anti-HER2 antibody-drug conjugate and the ATM inhibitor simultaneously;
[49] a method of treating cancer comprising administering an anti-HER2 antibody-drug conjugate and an ATM inhibitor as defined in any one of [1] to [15] in combination to a subject in need thereof;
[50] the method according to [49], wherein the cancer is as defined in any one of [19] to [34];
[51] the method according to [49] or [50], wherein the method comprises administering the anti-HER2 antibody-drug conjugate and the ATM inhibitor sequentially; and
[52] the method according to [49] or [50], wherein the method comprises administering the anti-HER2 antibody-drug conjugate and the ATM inhibitor simultaneously.

### Advantageous Effects of Disclosure

The present disclosure provides a pharmaceutical product wherein an anti-HER2 antibody-drug conjugate, having an antitumor drug conjugated to an anti-HER2 antibody via a linker structure, and an ATM inhibitor are administered in combination, and a therapeutic use and method wherein the specific antibody-drug conjugate and the ATM inhibitor are administered in combination to a subject. Thus, the present disclosure can provide a medicine and treatment which can obtain a superior antitumor effect in the treatment of cancers.

### BRIEF DESCRIPTION OF DRAWINGS

FIG. 1 is a diagram showing the amino acid sequence of a heavy chain of an anti-HER2 antibody (SEQ ID NO: 1).

FIG. 2 is a diagram showing the amino acid sequence of a light chain of an anti-HER2 antibody (SEQ ID NO: 2).

FIG. 3 is a diagram showing the amino acid sequence of a heavy chain CDRH1 (SEQ ID NO: 3 [=amino acid residues 26 to 33 of SEQ ID NO: 1]).

FIG. 4 is a diagram showing the amino acid sequence of a heavy chain CDRH2 (SEQ ID NO: 4 [=amino acid residues 51 to 58 of SEQ ID NO: 1]).

FIG. 5 is a diagram showing the amino acid sequence of a heavy chain CDRH3 (SEQ ID NO: 5 [=amino acid residues 97 to 109 of SEQ ID NO: 1]).

FIG. 6 is a diagram showing the amino acid sequence of a light chain CDRL1 (SEQ ID NO: 6 [=amino acid residues 27 to 32 of SEQ ID NO: 2]).

FIG. 7 is a diagram showing an amino acid sequence comprising the amino acid sequence of a light chain CDRL2 (SAS) (SEQ ID NO: 7 [=amino acid residues 50 to 56 of SEQ ID NO: 2]).

FIG. 8 is a diagram showing the amino acid sequence of a light chain CDRL3 (SEQ ID NO: 8 [=amino acid residues 89 to 97 of SEQ ID NO: 2]).

FIG. 9 is a diagram showing the amino acid sequence of a heavy chain variable region (SEQ ID NO: 9 [=amino acid residues 1 to 120 of SEQ ID NO: 1]).

FIG. 10 is a diagram showing the amino acid sequence of a light chain variable region (SEQ ID NO: 10 [=amino acid residues 1 to 107 of SEQ ID NO: 2]).

FIG. 11 is a diagram showing the amino acid sequence of a heavy chain (SEQ ID NO: 11 [=amino acid residues 1 to 449 of SEQ ID NO: 1]).

[FIGS. 12A and 12B] FIGS. 12A and 12B are diagrams showing combination matrices obtained with high-throughput screens combining DS-8201 with AZD1390 (AZ13791971; ATM inhibitor) in three breast cancer cell lines with diverse HER2 expression and one gastric cell line with high HER2 expression.

FIG. 13 is a diagram showing synergy matrices for combinations with DS-8201 and AZD1390 in HER2-high KPL4 cell line, in terms of (A) relative total cell counts as percentage of control, and (B) Loewe, Bliss and HSA scores.

FIG. 14 is a diagram showing change in total cells remaining after treatment compared to time zero for combinations of DS-8201 with AZD1390 in (A) HER2-high KPL4 cell line and (B) HER2-negative MDA-MB-468 cell line.

FIG. 15 is a diagram showing induction of ATM-dependent KAP1 pSer824 signalling, DNA double strand break damage (γH2AX) biomarkers or percentage of cell number (vs solvent control) for combinations of DS-8201 with AZD1390 in (A) HER2-high KPL4 cell line or (B) HER2-low MDA-MB-468 cell line.

FIG. 16 is a diagram showing change in tumour volume over time for treatment groups of female nude mice having NCI-N87 tumours implanted subcutaneously, treated with DS-8201 at 1 mg/kg or 3 mg/kg alone and in combination with AZD1390 at 10 mg/kg.

FIG. 17 is a diagram showing antibody blot images combining DS-8201 or exatecan mesylate, with AZD1390 in (A) NCI-N87 (gastric cancer) and (B) KPL4 (breast carcinoma) cell lines.

FIG. 18 In FIG. 18, (A) and (B) are diagrams showing combination matrices obtained with high-throughput screens combining DS-8201 with AZD1390 (ATM inhibitor) in HER2-high NCI-H2170 (lung cancer) cell line.

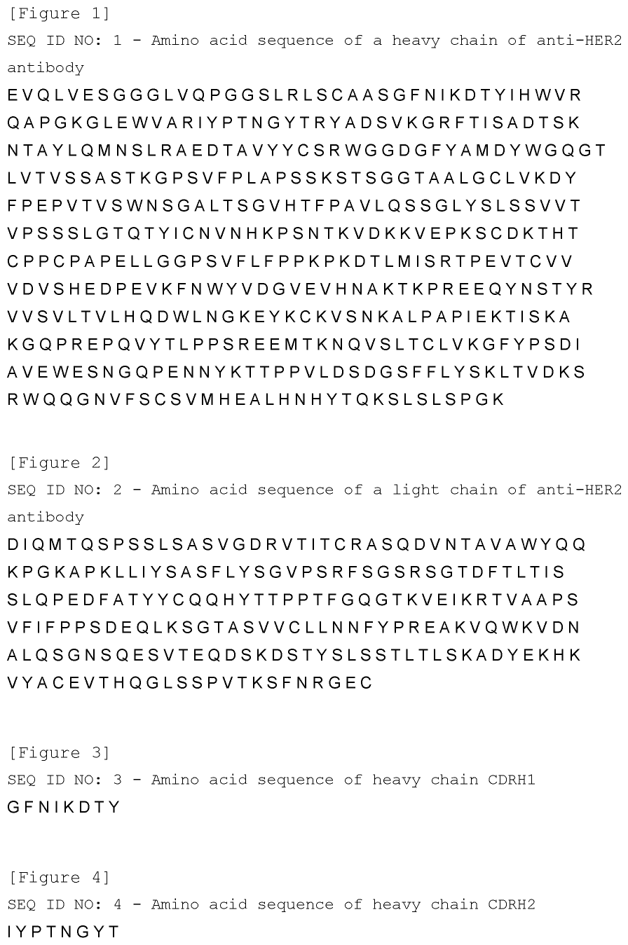

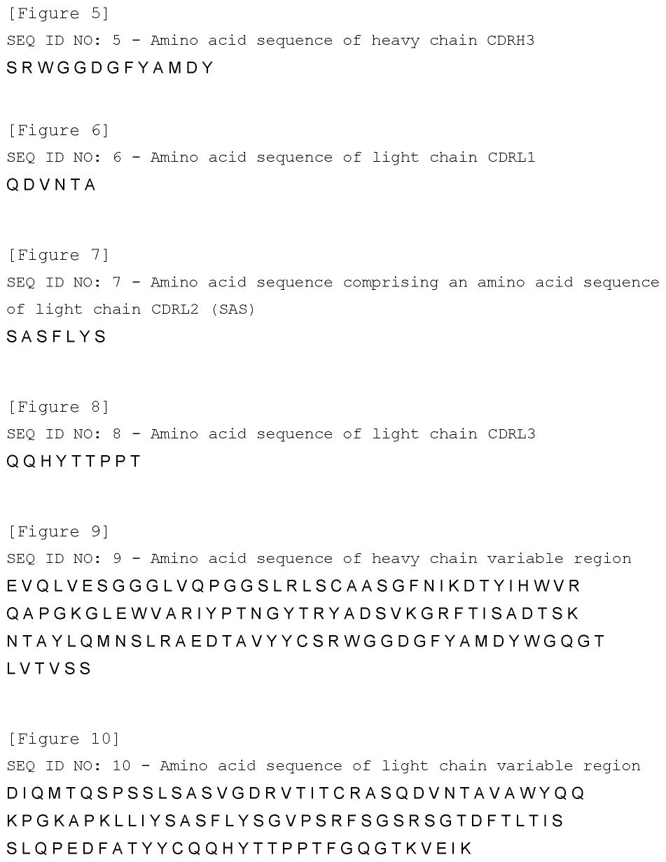

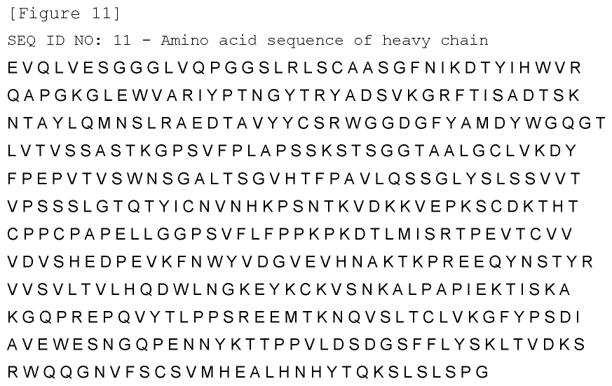

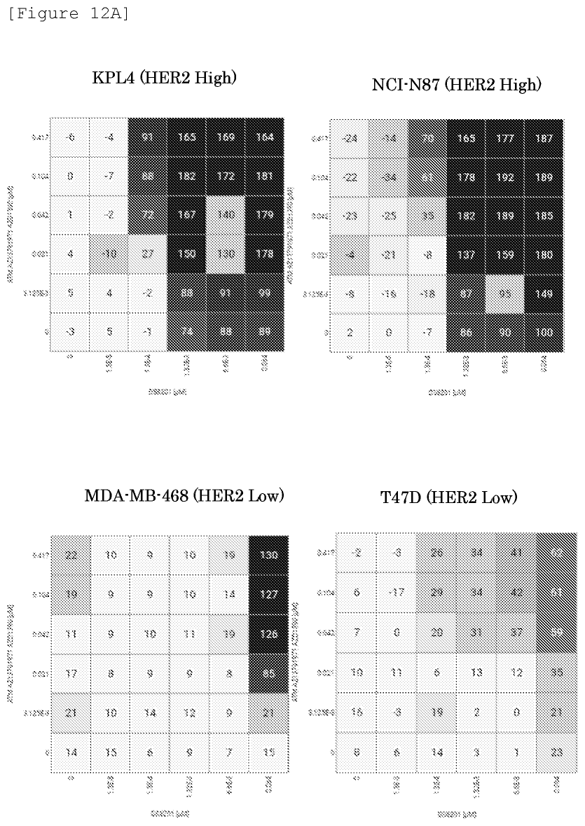

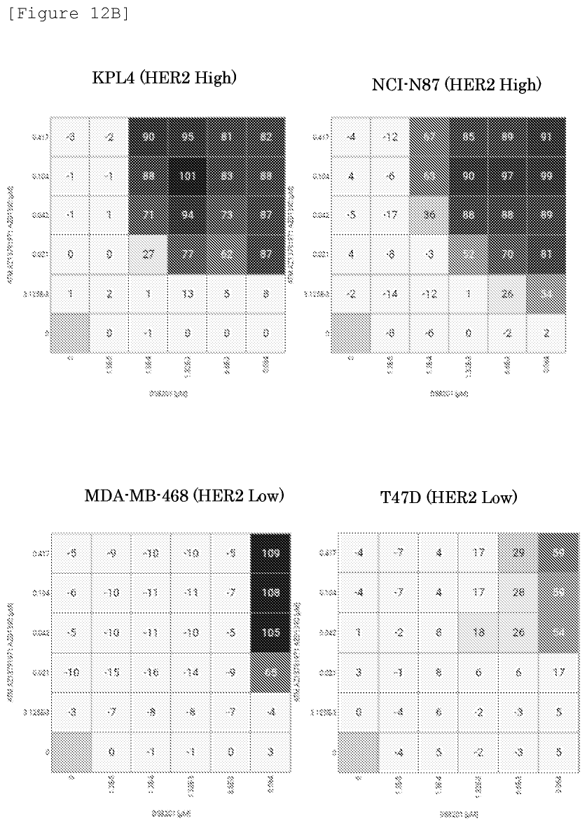

In order that the present disclosure can be more readily understood, certain terms are first defined. Additional definitions are set forth throughout the detailed description.

Before describing the present disclosure in detail, it is to be understood that this disclosure is not limited to specific compositions or method steps, as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

It is understood that wherever aspects are described herein with the language “comprising”, otherwise analogous aspects described in terms of “consisting of” and/or “consisting essentially of” are also provided. The terms “inhibit”, “block”, and “suppress” are used interchangeably herein and refer to any statistically significant decrease in biological activity, including full blocking of the activity. For example, “inhibition” can refer to a decrease of about 10%, 20%, 30%, 40%, 50%, 60%, 70%, 80%, 90% or 100% in biological activity. Cellular proliferation can be assayed using art recognized techniques which measure rate of cell division, and/or the fraction of cells within a cell population undergoing cell division, and/or rate of cell loss from a cell population due to terminal differentiation or cell death (e.g., thymidine incorporation).

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical product” refers to a preparation which is in such form as to permit the biological activity of the active ingredients, either as a composition containing all the active ingredients (for simultaneous administration), or as a combination of separate compositions (a combined preparation) each containing at least one but not all of the active ingredients (for administration sequentially or simultaneously), and which contains no additional components which are unacceptably toxic to a subject to which the product would be administered. Such product can be sterile. By “simultaneous administration” is meant that the active ingredients are administered at the same time. By “sequential administration” is meant that the active ingredients are administered one after the other, in either order, at a time interval between the individual administrations. The time interval can be, for example, less than 24 hours, preferably less than 6 hours, more preferably less than 2 hours.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” for cancer according to the methods of the present disclosure if the patient shows, e.g., total, partial, or transient remission of a certain type of cancer.

The terms “cancer”, “tumor”, “cancerous”, and “malignant” refer to or describe the physiological condition in mammals that is typically characterized by unregulated cell growth. Examples of cancers include but are not limited to, breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioma, glioblastoma multiforme, osteosarcoma, sarcoma, and melanoma. Cancers include hematological malignancies such as acute myeloid leukemia, multiple myeloma, chronic lymphocytic leukemia, diffuse large B cell lymphoma, Burkitt's lymphoma, follicular lymphoma and solid tumors such as breast cancer, lung cancer, neuroblastoma and colon cancer.

The term “cytotoxic agent” as used herein is defined broadly and refers to a substance that inhibits or prevents the function of cells and/or causes destruction of cells (cell death), and/or exerts anti-neoplastic/anti-proliferative effects. For example, a cytotoxic agent prevents directly or indirectly the development, maturation, or spread of neoplastic tumor cells. The term includes also such agents that cause a cytostatic effect only and not a mere cytotoxic effect. The term includes chemotherapeutic agents as specified below, as well as other HER2 antagonists, anti-angiogenic agents, tyrosine kinase inhibitors, protein kinase A inhibitors, members of the cytokine family, radioactive isotopes, and toxins such as enzymatically active toxins of bacterial, fungal, plant or animal origin.

The term “chemotherapeutic agent” is a subset of the term “cytotoxic agent” comprising natural or synthetic chemical compounds.

In accordance with the methods or uses of the present disclosure, compounds of the present disclosure may be administered to a patient to promote a positive therapeutic response with respect to cancer. The term “positive therapeutic response” with respect to cancer treatment refers to an improvement in the symptoms associated with the disease. For example, an improvement in the disease can be characterized as a complete response. The term “complete response” refers to an absence of clinically detectable disease with normalization of any previous test results. Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of compounds of the present disclosure. In specific aspects, such terms refer to one, two or three or more results following the administration of compounds of the instant disclosure: (1) a stabilization, reduction or elimination of the cancer cell population;

(2) a stabilization or reduction in cancer growth;
(3) an impairment in the formation of cancer;
(4) eradication, removal, or control of primary, regional and/or metastatic cancer;
(5) a reduction in mortality;
(6) an increase in disease-free, relapse-free, progression-free, and/or overall survival, duration, or rate;
(7) an increase in the response rate, the durability of response, or number of patients who respond or are in remission;
(8) a decrease in hospitalization rate,
(9) a decrease in hospitalization lengths,
(10) the size of the cancer is maintained and does not increase or increases by less than 10%, preferably less than 5%, preferably less than 4%, preferably less than 2%, and
(11) an increase in the number of patients in remission.
(12) a decrease in the number of adjuvant therapies (e.g., chemotherapy or hormonal therapy) that would otherwise be required to treat the cancer.

Clinical response can be assessed using screening techniques such as PET, magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy can experience the beneficial effect of an improvement in the symptoms associated with the disease.

As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical product will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In particular, an effective amount of a compound of formula (I) for use in the treatment of cancer in combination with the antibody-drug conjugate is an amount such that the combination is sufficient to symptomatically relieve in a warm-blooded animal such as man, the symptoms of cancer, to slow the progression of cancer, or to reduce in patients with symptoms of cancer the risk of getting worse.

Where the term “optionally” is used, it is intended that the subsequent feature may or may not occur. As such, use of the term “optionally” includes instances where the feature is present, and also instances where the feature is not present. For example, a group “optionally substituted by one methoxy group” includes groups with and without a methoxy substituent.

The term “substituted” means that one or more hydrogens (for example 1 or 2 hydrogens, or alternatively 1 hydrogen) on the designated group is replaced by the indicated substituent(s) (for example 1 or 2 substituents, or alternatively 1 substituent), provided that any atom(s) bearing a substituent maintains a permitted valency. Substituent combinations encompass only stable compounds and stable synthetic intermediates. “Stable” means that the relevant compound or intermediate is sufficiently robust to be isolated and have utility either as a synthetic intermediate or as an agent having potential therapeutic utility. If a group is not described as “substituted”, or “optionally substituted”, it is to be regarded as unsubstituted (i.e. that none of the hydrogens on the designated group have been replaced).

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich:Wiley-VCH/VHCA, 2002. A suitable pharmaceutically acceptable salt of a compound of formula (I) or (II) is, for example, an acid-addition salt. An acid addition salt of a compound of formula (I) or (II) may be formed by bringing the compound into contact with a suitable inorganic or organic acid under conditions known to the skilled person. An acid addition salt may for example be formed using an inorganic acid selected from the group consisting of hydrochloric acid, hydrobromic acid, sulphuric acid and phosphoric acid. An acid addition salt may also be formed using an organic acid selected from the group consisting of trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid.

Therefore, in one embodiment of a compound of formula (I) or (II) or a pharmaceutically acceptable salt thereof, the pharmaceutically acceptable salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt. In another embodiment of a compound of formula (I) or (II) or a pharmaceutically acceptable salt thereof, the pharmaceutically acceptable salt is a methanesulfonic acid salt. In another embodiment of a compound of formula (I) or (II) or a pharmaceutically acceptable salt thereof, the pharmaceutically acceptable salt is a mono-methanesulfonic acid salt, i.e. the stoichiometry of the compound of the compound of formula (I) or (II) to methanesulfonic acid is 1:1.

Compounds and salts described in this specification may exist in solvated forms and unsolvated forms. For example, a solvated form may be a hydrated form, such as a hemi-hydrate, a mono-hydrate, a di-hydrate, a tri-hydrate or an alternative quantity thereof. The disclosure encompasses all such solvated and unsolvated forms of compounds of formula (I) or (II), particularly to the extent that such forms possess ATM kinase inhibitory activity.

Atoms of the compounds and salts described in this specification may exist as their isotopes. The disclosure encompasses all compounds of formula (I) or (II) where an atom is replaced by one or more of its isotopes (for example a compound of formula (I) or (II) where one or more carbon atom is an ¹¹C or ¹³C carbon isotope, or where one or more hydrogen atoms is a ²H or ³H isotope, or where one of more fluorine atoms is an ¹⁸F isotope).

Compounds and salts described in this specification may exist as a mixture of tautomers. “Tautomers” are structural isomers that exist in equilibrium resulting from the migration of a hydrogen atom. The disclosure includes all tautomers of compounds of formula (I) or (II) particularly to the extent that such tautomers possess ATM kinase inhibitory activity.

Compounds and salts described in this specification may exist in optically active or racemic forms by virtue of one or more asymmetric carbon atoms. The disclosure includes any optically active or racemic form of a compound of formula (I) or (II) which possesses ATM kinase inhibitory activity. The synthesis of optically active forms may be carried out by standard techniques of organic chemistry well known in the art, for example by synthesis using optically active materials or by resolution of a racemic form.

Therefore, in one embodiment of a compound of formula (I) or (II), or a pharmaceutically acceptable salt thereof, the compound is a single optical isomer being in an enantiomeric excess (% ee) of ≥95%, ≥98% or ≥99%. In another embodiment, the single optical isomer is present in an enantiomeric excess (% ee) of ≥99%.

Compounds and salts described in this specification may be crystalline, and may exhibit one or more crystalline forms. The disclosure encompasses any crystalline or amorphous form of a compound of formula (I) or (II), or mixture of such forms, which possesses ATM kinase inhibitory activity.

### Description of Embodiments

Hereinafter, preferred modes for carrying out the present disclosure are described. The embodiments described below are given merely for illustrating one example of a typical embodiment of the present disclosure and are not intended to limit the scope of the present disclosure.

### 1. Antibody-Drug Conjugate

The antibody-drug conjugate used in the present disclosure is an antibody-drug conjugate in which a drug-linker represented by the following formula:

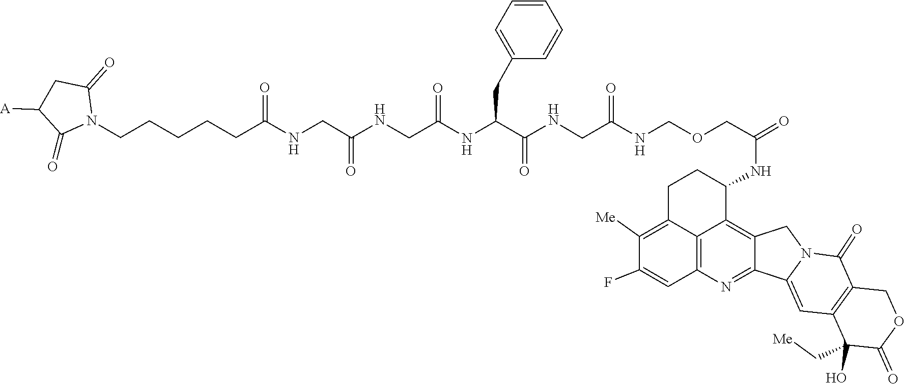

wherein A represents the connecting position to an antibody,
is conjugated to an anti-HER2 antibody via a thioether bond.

In the present disclosure, the partial structure consisting of a linker and a drug in the antibody-drug conjugate is referred to as a “drug-linker”. The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of the present disclosure includes exatecan (IUPAC name: (1S,9S)-1-amino-9-ethyl-5-fluoro-1,2,3,9,12,15-hexahydro-9-hydroxy-4-methyl-10H,13H-benzo[de]pyrano[3′,4′:6,7]indolizino[1,2-b]quinolin-10,13-dione, (also expressed as chemical name: (1S,9S)-1-amino-9-ethyl-5-fluoro-2,3-dihydro-9-hydroxy-4-methyl-1H,12H-benzo[de]pyrano[3′,4′:6,7]indolizino[1,2-b]quinolin-10,13(9H,15H)-dione)), which is a topoisomerase I inhibitor, as a component. Exatecan is a camptothecin derivative having an antitumor effect, represented by the following formula:

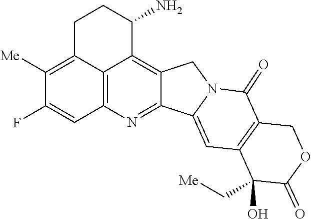 [SMILES: CC[C@@]1(O)C(=O)OCc2c1cc1n(c2=O)Cc2c-1nc1cc(F)c(C)c3c1c2[C@@H](N)CC3]

The anti-HER2 antibody-drug conjugate used in the present disclosure can be also represented by the following formula:

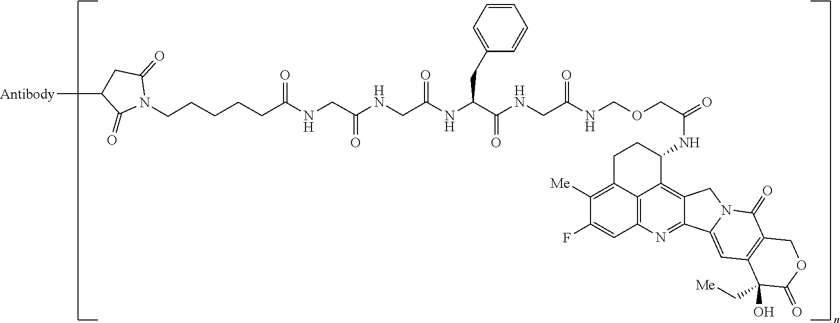

Here, the drug-linker is conjugated to an anti-HER2 antibody (‘Antibody-’) via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, the anti-HER2 antibody-drug conjugate used in the present disclosure is cleaved at the linker portion to release a compound represented by the following formula:

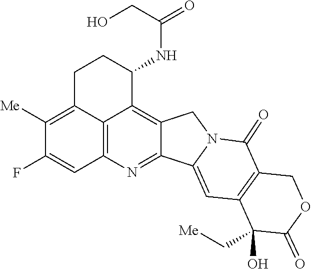 [SMILES: CC[C@@]1(O)C(=O)OCc2c1cc1n(c2=O)Cc2c-1nc1cc(F)c(C)c3c1c2[C@@H](NC(=O)CO)CC3]

This compound is inferred to be the original source of the antitumor activity of the antibody-drug conjugate used in the present disclosure, and has been confirmed to have a topoisomerase I inhibitory effect (Ogitani Y. et al., Clinical Cancer Research, 2016, Oct. 15;22(20):5097-5108, Epub 2016 Mar. 29).

The anti-HER2 antibody-drug conjugate used in the present disclosure is known to have a bystander effect (Ogitani Y. et al., Cancer Science (2016) 107, 1039-1046). The bystander effect is exerted through a process whereby the antibody-drug conjugate used in the present disclosure is internalized in cancer cells expressing the target and the compound released then exerts an antitumor effect also on cancer cells which are present therearound and not expressing the target. This bystander effect is exerted as an excellent antitumor effect even when the anti-HER2 antibody-drug conjugate is used in combination with an ATM inhibitor according to the present disclosure.

### 2. Antibody in Antibody-Drug Conjugate

The anti-HER2 antibody in the antibody-drug conjugate used in the present disclosure may be derived from any species, and is preferably an anti-HER2 antibody derived from a human, a rat, a mouse, or a rabbit. In cases when the antibody is derived from species other than human species, it is preferably chimerized or humanized using a well known technique. The anti-HER2 antibody may be a polyclonal antibody or a monoclonal antibody and is preferably a monoclonal antibody.

The antibody in the antibody-drug conjugate used in the present disclosure is an anti-HER2 antibody preferably having a characteristic of being capable of targeting cancer cells, and is preferably an antibody possessing, for example, a property of recognizing a cancer cell, a property of binding to a cancer cell, a property of internalizing in a cancer cell, and/or cytocidal activity against cancer cells.

The binding activity of the anti-HER2 antibody against cancer cells can be confirmed using flow cytometry. The internalization of the antibody into cancer cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282, December 2004), or (3) a Mab-ZAP assay using an immunotoxin binding to the therapeutic antibody wherein the toxin is released upon incorporation into cells to inhibit cell growth (Bio Techniques 28: 162-165, January 2000). As the immunotoxin, a recombinant complex protein of a diphtheria toxin catalytic domain and protein G may be used.

The antitumor activity of the anti-HER2 antibody can be confirmed in vitro by determining inhibitory activity against cell growth. For example, a cancer cell line overexpressing HER2 as a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining change in the cancer cell.

Since the compound conjugated in the anti-HER2 antibody-drug conjugate exerts an antitumor effect, it is preferred but not essential that the anti-HER2 antibody itself should have an antitumor effect. For the purpose of specifically and selectively exerting the cytotoxic activity of the antitumor compound against cancer cells, it is important and also preferred that the anti-HER2 antibody should have the property of internalizing to migrate into cancer cells.

The anti-HER2 antibody in the antibody-drug conjugate used in the present disclosure can be obtained by a procedure known in the art. For example, the antibody of the present disclosure can be obtained using a method usually carried out in the art, which involves immunizing animals with an antigenic polypeptide and collecting and purifying antibodies produced in vivo.

The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen are fused with myeloma cells according to a method known in the art (e.g., Kohler and Milstein, Nature (1975) 256, p. 495-497; and Kennet, R. ed., Monoclonal Antibodies, p. 365-367, Plenum Press, N.Y. (1980)) to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The anti-HER2 antibody in the antibody-drug conjugate used the present disclosure is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), and an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p.133-143; Kuroiwa, Y. et. al., Nucl. Acids Res. (1998) 26, p.3447-3448; Yoshida, H. et. al., Animal Cell Technology:Basic and Applied Aspects vol. 10, p.69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p.722-727, etc.) can be exemplified. As an alternative, an antibody obtained by phage display, the antibody being selected from a human antibody library (see Wormstone, I. M. et. al, Investigative Ophthalmology & Visual Science. (2002)43 (7), p.2301-2308; Carmen, S. et. al., Briefings in Functional Genomics and Proteomics (2002), 1(2), p.189-203; Siriwardena, D. et. al., Ophthalmology (2002) 109(3), p.427-431, etc.) can be exemplified.

In the present disclosure, modified variants of the anti-HER2 antibody in the antibody-drug conjugate used in the present disclosure are also included. The modified variant refers to a variant obtained by subjecting the antibody according to the present disclosure to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present disclosure, for example, an enzyme-labeled antibody, a fluorescence-labeled antibody, and an affinity-labeled antibody are also included in the meaning of the modified variant. Such a modified variant of the antibody according to the present disclosure is useful for improving the stability and blood retention of the antibody, reducing the antigenicity thereof, detecting or isolating an antibody or an antigen, and so on.

Further, by regulating the modification of a glycan which is linked to the antibody according to the present disclosure (glycosylation, defucosylation, etc.), it is possible to enhance antibody-dependent cellular cytotoxic activity. As the technique for regulating the modification of a glycan of antibodies, those disclosed in WO99/54342, WO00/61739, WO02/31140, WO2007/133855, WO2013/120066, etc. are known. However, the technique is not limited thereto. In the anti-HER2 antibody according to the present disclosure, antibodies in which the modification of a glycan is regulated are also included.

It is known that a lysine residue at the carboxyl terminus of the heavy chain of an antibody produced in a cultured mammalian cell is deleted (Journal of Chromatography A, 705: 129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360: 75-83 (2007)). However, such deletion and modification of the heavy chain sequence do not affect the antigen-binding affinity and the effector function (the activation of complement, antibody-dependent cellular cytotoxicity, etc.) of the antibody. Therefore, in the anti-HER2 antibody according to the present disclosure, antibodies subjected to such modification and functional fragments of the antibody are also included, and deletion variants in which one or two amino acids have been deleted at the carboxyl terminus of the heavy chain, variants obtained by amidation of deletion variants (for example, a heavy chain in which the carboxyl terminal proline residue has been amidated), and the like are also included. The type of deletion variant having a deletion at the carboxyl terminus of the heavy chain of the anti-HER2 antibody according to the present disclosure is not limited to the above variants as long as the antigen-binding affinity and the effector function are conserved. The two heavy chains constituting the antibody according to the present disclosure may be of one type selected from the group consisting of a full-length heavy chain and the above-described deletion variant, or may be of two types in combination selected therefrom. The ratio of the amount of each deletion variant can be affected by the type of cultured mammalian cells which produce the anti-HER2 antibody according to the present disclosure and the culture conditions; however, an antibody in which one amino acid residue at the carboxyl terminus has been deleted in both of the two heavy chains in the antibody according to the present disclosure can be exemplified as preferred.

As isotypes of the anti-HER2 antibody according to the present disclosure, for example, IgG (IgG1, IgG2, IgG3, IgG4) can be exemplified, and IgG1 or IgG2 can be exemplified as preferred.

In the present disclosure, the term “anti-HER2 antibody” refers to an antibody which specifically binds to HER2 (Human Epidermal Growth Factor Receptor Type 2; ErbB-2), and preferably has an activity of internalizing in HER2-expressing cells by binding to HER2.

Examples of the anti-HER2 antibody include trastuzumab (U.S. Pat. No. 5,821,337) and pertuzumab (WO01/00245), and trastuzumab can be exemplified as preferred.

### 3. Production of Antibody-Drug Conjugate

A drug-linker intermediate for use in production of the anti-HER2 antibody-drug conjugate according to the present disclosure is represented by the following formula:

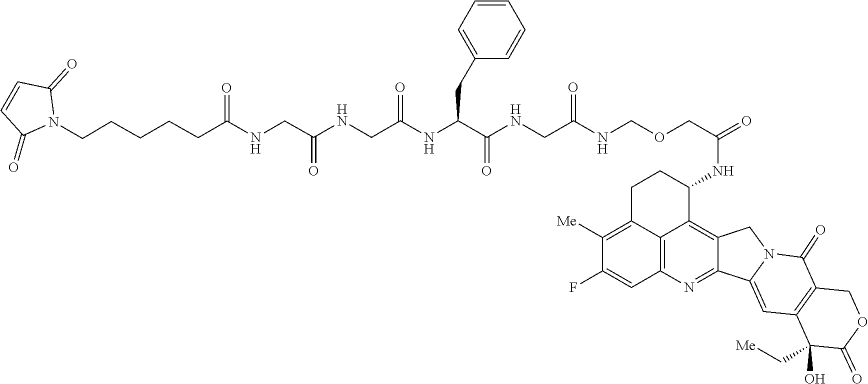 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl)hexanoyl]glycylglycyl-L-phenylalanyl-N-[(2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethoxy)methyl]glycinamide, and can be produced with reference to descriptions in WO2014/057687, WO2015/098099, WO2015/115091, WO2015/155998, WO2019/044947 and so on.

The anti-HER2 antibody-drug conjugate used in the present disclosure can be produced by reacting the above-described drug-linker intermediate and an anti-HER2 antibody having a thiol group (also referred to as a sulfhydryl group).

The anti-HER2 antibody having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl)phosphine hydrochloride (TCEP) per interchain disulfide within the antibody and reacting with the antibody in a buffer solution containing a chelating agent such as ethylenediamine tetraacetic acid (EDTA), an anti-HER2 antibody having a sulfhydryl group with partially or completely reduced interchain disulfides within the antibody can be obtained.

Further, by using 2 to 20 molar equivalents of the drug-linker intermediate per anti-HER2 antibody having a sulfhydryl group, an anti-HER2 antibody-drug conjugate in which 2 to 8 drug molecules are conjugated per antibody molecule can be produced.

The average number of conjugated drug molecules per anti-HER2 antibody molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the anti-HER2 antibody and the drug-linker intermediate and calculation of the average number of conjugated drug molecules per antibody molecule of the antibody-drug conjugate can be performed with reference to descriptions in WO2014/057687, WO2015/098099, WO2015/115091, WO2015/155998, WO2017/002776, WO2018/212136, and so on.

In the present disclosure, the term “anti-HER2 antibody-drug conjugate” refers to an antibody-drug conjugate such that the antibody in the antibody-drug conjugate according to the present disclosure is an anti-HER2 antibody.

The anti-HER2 antibody is preferably an antibody comprising a heavy chain comprising CDRH1 consisting of an amino acid sequence consisting of amino acid residues 26 to 33 of SEQ ID NO: 1, CDRH2 consisting of an amino acid sequence consisting of amino acid residues 51 to 58 of SEQ ID NO: 1 and CDRH3 consisting of an amino acid sequence consisting of amino acid residues 97 to 109 of SEQ ID NO: 1, and a light chain comprising CDRL1 consisting of an amino acid sequence consisting of amino acid residues 27 to 32 of SEQ ID NO: 2, CDRL2 consisting of an amino acid sequence consisting of amino acid residues 50 to 52 of SEQ ID NO: 2 and CDRL3 consisting of an amino acid sequence consisting of amino acid residues 89 to 97 of SEQ ID NO: 2, and more preferably an antibody comprising a heavy chain comprising a heavy chain variable region consisting of an amino acid sequence consisting of amino acid residues 1 to 120 of SEQ ID NO: 1 and a light chain comprising a light chain variable region consisting of an amino acid sequence consisting of amino acid residues 1 to 107 of SEQ ID NO: 2, and even more preferably an antibody comprising a heavy chain consisting of an amino acid sequence represented by SEQ ID NO: 1 and a light chain consisting of the amino acid sequence represented by SEQ ID NO: 2, or an antibody comprising a heavy chain consisting of amino acid residues 1 to 449 of SEQ ID NO: 1 and a light chain consisting of an amino acid sequence consisting of all amino acid residues 1 to 214 of SEQ ID NO: 2.

The average number of units of the drug-linker conjugated per antibody molecule in the anti-HER2 antibody-drug conjugate is preferably 2 to 8, more preferably 3 to 8, even more preferably 7 to 8, even more preferably 7.5 to 8, and even more preferably about 8.

The anti-HER2 antibody-drug conjugate used in the present disclosure can be produced with reference to descriptions in WO2015/115091 and so on.

In preferred embodiments, the anti-HER2 antibody-drug conjugate is trastuzumab deruxtecan (DS-8201).

### 4. ATM Inhibitor

In the present disclosure, the term “ATM inhibitor” refers to an agent that inhibits ATM (ataxia telangiectasia mutated kinase). The ATM inhibitor in the present disclosure may selectively inhibit the kinase ATM, or may non-selectively inhibit ATM and inhibit also kinase(s) other than ATM. Preferably, the ATM inhibitor in the present disclosure inhibits ATM selectively. The ATM inhibitor in the present disclosure is not particularly limited as long as it is an agent that has the described characteristics, and preferred examples thereof can include those disclosed in WO2017/046216, WO2015/170081, WO2018/167203, WO2017/153578, WO2017/162611, WO2017/162605, WO2017/174446, WO2017/076895, WO2017/076898, WO2017/194632, WO2019/057757.

In other embodiments of the ATM inhibitor used in the present disclosure, the ATM inhibitor is a compound selected from:

### AZD1390:

7-fluoro-1-isopropyl-3-methyl-8-[6-[3-(1-piperidyl)propoxy]-3-pyridyl]imidazo[4,5-c]quinolin-2-one (WO2017/046216: Example 2), and its deuterated forms: 4,6-Dideutero-7-fluoro-1-isopropyl-3-methyl-8-[6-[3-(1-piperidyl)propoxy]-3-pyridyl]imidazo[4,5-c]quinolin-2-one and
4-Deutero-7-fluoro-1-isopropyl-3-methyl-8-[6-[3-(1-piperidyl)propoxy]-3-pyridyl]imidazo[4,5-c]quinolin-2-one;

### AZD0156

8-[6-(3-dimethylaminopropoxy)pyridin-3-yl]-3-methyl-1-(oxan-4-yl)imidazo[5,4-c]quinolin-2-one (WO2015/170081: Example 1);

### M4076

8-(1,3-dimethyl-1H-pyrazol-4-yl)-1-(3-fluoro-5-methoxy-pyridin-4-yl)-7-methoxy-3-methyl-1,3-dihydroimidazo[4,5-c]quinolin-2-one (WO2016/155884);

### M3541

3-fluoro-4-[7-methoxy-3-methyl-8-(1-methyl-1H-pyrazol-4-yl)-2-oxo-2,3-dihydro-1H-imidazo[4,5-c]quinolin-1-yl]benzonitrile (WO2012/028233);

### KU-55933

2-(4-morpholinyl)-6-(1-thianthrenyl)-4H-pyran-4-one (CAS number 587871-26-9; WO2003070726A1);

### KU-60019:

2-[(2R,6S)-2,6-dimethyl-4-morpholinyl]-N-{5-[6-(4-morpholinyl)-4-oxo-4H-pyran-2-yl]-9H-thioxanthen-2-yl}acetamide (CAS number 925701-49-1);

### KU-59403

3-(4-methyl-1-piperazinyl)-N-{6-[6-(4-morpholinyl)-4-oxo-4H-pyran-2-yl]-2-thianthrenyl}propanamide (WO2005016919);

### CP466722

2-(6,7-dimethoxyquinazolin-4-yl)-5-(2-pyridyl)-1,2,4-triazol-3-amine (CAS number 1080622-86-1);

### NVP-BEZ235

2-methyl-2-[4-[3-methyl-2-oxo-8-(3-quinolyl)imidazo[4,5-c]quinolin-1-yl]phenyl]propanenitrile (dactolisib; CAS number 915019-65-7; WO2006/122806);
   6-[6-(methoxymethyl)-3-pyridyl]-4-[[(1S)-1-tetrahydropyran-4-ylethyl]amino]quinoline-3-carboxamide (Degorce et al, J Med chem, 2016, 59, 6281);
   7-fluoro-6-[6-(methoxymethyl)-3-pyridyl]-4-[[(1S)-1-(1-methylpyrazol-3-yl)ethyl]amino]quinoline-3-carboxamide (Degorce et al, J Med chem, 2016, 59, 6281);
6-[6-[3-(dimethylamino)propoxy]-3-pyridyl]-N-methyl-4-[[(1S)-1-tetrahydropyran-4-ylethyl]amino]cinnoline-3-carboxamide (Barlaam et al, Med Chem Lett, 2018, 9, 809-814);
N-methyl-4-(6-phenylimidazo[1,2-a]pyrazin-3-yl)benzamide (Valerie et al, Mol Can Ther, 2018, 1637);
1-isopropyl-3-methyl-8-[6-[3-(1-piperidyl)propoxy]-3-pyridyl]imidazo[4,5-c]cinnolin-2-one (WO2019/057757); and
2-(7-methoxy-9H-thioxanthen-4-yl)-6-morpholino-pyran-4-one (J. Med. Chem, 2019, 62, 2988).

According to preferred embodiments of the ATM inhibitor used in the present disclosure, the ATM inhibitor is a compound represented by the following formula (I):

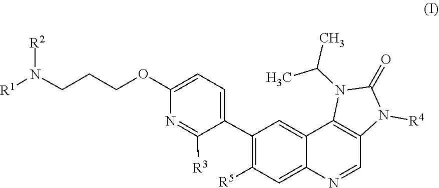

where:

R¹ is methyl;

R² is hydrogen or methyl; or R¹ and R² together with the nitrogen atom to which they are bonded form an azetidinyl, pyrrolidinyl or piperidinyl ring;

R³ is hydrogen or fluoro;

R⁴ is hydrogen or methyl; and

R⁵ is hydrogen or fluoro, or a pharmaceutically acceptable salt thereof.

Where it is mentioned that “R¹ and R² together with the nitrogen atom to which they are bonded form an azetidinyl, pyrrolidinyl or piperidinyl ring”, this means the R¹ and R² groups are joined via a carbon-carbon covalent bond to form an unsubstituted alkylene chain of the appropriate length to form the corresponding ring. For example, when R¹ and R² together with the nitrogen atom to which they are bonded form a pyrrolidinyl ring, R¹ and R² together represent an unsubstituted butylene chain which is attached to the relevant nitrogen atom in formula (I) at both terminal carbons.

Some values of variable groups in formula (I) are as follows. Such values may be used in combination with any of the definitions, claims, or embodiments defined herein to provide further embodiments of compounds of formula (I):
a) R¹ is methyl.
b) R² is methyl.
c) R² is hydrogen.
d) R¹ is methyl and R² is hydrogen or methyl.
e) R¹ and R² are both methyl.
f) R¹ and R² are both methyl; or R¹ and R² together with the nitrogen atom to which they are bonded form an azetidinyl, pyrrolidinyl or piperidinyl ring.
g) R¹ and R² are both methyl; or R¹ and R² together with the nitrogen atom to which they are bonded form an azetidinyl ring.
h) R¹ and R² are both methyl; or R¹ and R² together with the nitrogen atom to which they are bonded form a pyrrolidinyl ring.
i) R¹ and R² are both methyl; or R¹ and R² together with the nitrogen atom to which they are bonded form a piperidinyl ring.
j) R¹ and R² are both methyl.
k) R¹ and R² together with the nitrogen atom to which they are bonded form an azetidinyl, pyrrolidinyl or piperidinyl ring.
l) R¹ and R² together with the nitrogen atom to which they are bonded form an azetidinyl ring.
m) R¹ and R² together with the nitrogen atom to which they are bonded form a pyrrolidinyl ring.
n) R¹ and R² together with the nitrogen atom to which they are bonded form a piperidinyl ring.
o) R³ and R⁵ are both hydrogen.
p) R³ and R⁵ are both fluoro.
q) R³ is hydrogen.
r) R³ is fluoro.
s) R⁴ is hydrogen.
t) R⁴ is methyl.
u) R⁵ is hydrogen.
v) R⁵ is fluoro.

In one embodiment, the compound is represented by formula (I), or a pharmaceutically acceptable salt thereof, where:

R¹ is methyl;

R² is hydrogen or methyl; or R¹ and R² together with the nitrogen atom to which they are bonded form an azetidinyl, pyrrolidinyl or piperidinyl ring;

R³ is hydrogen or fluoro;

R⁴ is hydrogen or methyl; and

R⁵ is hydrogen or fluoro.

In another embodiment, the compound is represented by formula (I), or a pharmaceutically acceptable salt thereof, where:

R¹ is methyl;

R² is hydrogen or methyl;

R³ is hydrogen;

R⁴ is hydrogen or methyl; and

R⁵ is hydrogen.

In another embodiment, the compound is represented by formula (I), or a pharmaceutically acceptable salt thereof, where the compound is selected from the group consisting of:
8-[6-[3-(Dimethylamino)propoxy]-3-pyridyl]-7-fluoro-1-isopropyl-3-methyl-imidazo[4,5-c]quinolin-2-one;
7-Fluoro-1-isopropyl-3-methyl-8-[6-[3-(1-piperidyl)propoxy]-3-pyridyl]imidazo[4,5-c]quinolin-2-one;
7-Fluoro-1-isopropyl-3-methyl-8-[6-(3-pyrrolidin-1-ylpropoxy)-3-pyridyl]imidazo[4,5-c]quinolin-2-one;
8-[6-[3-(azetidin-1-yl)propoxy]-3-pyridyl]-7-fluoro-1-isopropyl-3-methyl-imidazo[4,5-c]quinolin-2-one;
1-Isopropyl-3-methyl-8-[6-[3-(1-piperidyl)propoxy]-3-pyridyl]imidazo[4,5-c]quinolin-2-one;
8-[6-[3-(dimethylamino)propoxy]-3-pyridyl]-1-isopropyl-3-methyl-imidazo[4,5-c]quinolin-2-one;
1-isopropyl-3-methyl-8-[6-(3-pyrrolidin-1-ylpropoxy)-3-pyridyl]imidazo[4,5-c]quinolin-2-one;
8-[6-[3-(Azetidin-1-yl)propoxy]-3-pyridyl]-1-isopropyl-3-methyl-imidazo[4,5-c]quinolin-2-one;
8-[2-Fluoro-6-[3-(1-piperidyl)propoxy]-3-pyridyl]-1-isopropyl-3-methyl-imidazo[4,5-c]quinolin-2-one;
8-[6-[3-(dimethylamino)propoxy]-2-fluoro-3-pyridyl]-1-isopropyl-3-methyl-imidazo[4,5-c]quinolin-2-one;
8-[6-[3-(dimethylamino)propoxy]-2-fluoro-3-pyridyl]-7-fluoro-1-isopropyl-3-methyl-imidazo[4,5-c]quinolin-2-one;
8-[2-fluoro-6-(3-pyrrolidin-1-ylpropoxy)-3-pyridyl]-1-isopropyl-3-methyl-imidazo[4,5-c]quinolin-2-one;
7-fluoro-8-[2-fluoro-6-(3-pyrrolidin-1-ylpropoxy)-3-pyridyl]-1-isopropyl-3-methyl-imidazo[4,5-c]quinolin-2-one;
7-fluoro-8-[2-fluoro-6-[3-(1-piperidyl)propoxy]-3-pyridyl]-1-isopropyl-3-methyl-imidazo[4,5-c]quinolin-2-one;
8-[6-[3-(azetidin-1-yl)propoxy]-2-fluoro-3-pyridyl]-1-isopropyl-3-methyl-imidazo[4,5-c]quinolin-2-one;
8-[6-[3-(azetidin-1-yl)propoxy]-2-fluoro-3-pyridyl]-7-fluoro-1-isopropyl-3-methyl-imidazo[4,5-c]quinolin-2-one;
8-[6-[3-(Dimethylamino)propoxy]-3-pyridyl]-7-fluoro-1-isopropyl-3H-imidazo[4,5-c]quinolin-2-one; and
7-Fluoro-1-isopropyl-8-[6-[3-(1-piperidyl)propoxy]-3-pyridyl]-3H-imidazo[4,5-c]quinolin-2-one.

In another embodiment, the compound represented by formula (I) is 7-fluoro-1-isopropyl-3-methyl-8-[6-[3-(1-piperidyl)propoxy]-3-pyridyl]imidazo[4,5-c]quinolin-2-one, or a pharmaceutically acceptable salt thereof.

In another embodiment, the compound represented by formula (I) is 7-fluoro-1-isopropyl-3-methyl-8-[6-[3-(1-piperidyl)propoxy]-3-pyridyl]imidazo[4,5-c]quinolin-2-one.

In another embodiment, the compound according to formula (I) is a pharmaceutically acceptable salt of 7-fluoro-1-isopropyl-3-methyl-8-[6-[3-(1-piperidyl)propoxy]-3-pyridyl]imidazo[4,5-c]quinolin-2-one.

In another embodiment, the compound represented by formula (I) is 8-[6-[3-(azetidin-1-yl)propoxy]-3-pyridyl]-1-isopropyl-3-methyl-imidazo[4,5-c]quinolin-2-one or a pharmaceutically acceptable salt thereof.

In another embodiment, the compound represented by formula (I) is 8-[6-[3-(azetidin-1-yl)propoxy]-3-pyridyl]-1-isopropyl-3-methyl-imidazo[4,5-c]quinolin-2-one.

In another embodiment, the compound according to formula (I) is a pharmaceutically acceptable salt of 8-[6-[3-(azetidin-1-yl)propoxy]-3-pyridyl]-1-isopropyl-3-methyl-imidazo[4,5-c]quinolin-2-one.

In another embodiment, the compound represented by formula (I) is 8-[6-[3-(dimethylamino)propoxy]-3-pyridyl]-7-fluoro-1-isopropyl-3-methyl-imidazo[4,5-c]quinolin-2-one, or a pharmaceutically acceptable salt thereof.

In another embodiment, the compound represented by formula (I) is 8-[6-[3-(dimethylamino)propoxy]-3-pyridyl]-7-fluoro-1-isopropyl-3-methyl-imidazo[4,5-c]quinolin-2-one.

In another embodiment, the compound according to formula (I) is a pharmaceutically acceptable salt of 8-[6-[3-(dimethylamino)propoxy]-3-pyridyl]-7-fluoro-1-isopropyl-3-methyl-imidazo[4,5-c]quinolin-2-one.

In another embodiment, the compound represented by formula (I) is 7-fluoro-1-isopropyl-3-methyl-8-[6-[3-(1-oxidopiperidin-1-ium-1-yl)propoxy]-3-pyridyl]imidazo[4,5-c]quinolin-2-one, or a pharmaceutically acceptable salt thereof.

In another embodiment, the compound represented by formula (I) is 7-fluoro-1-isopropyl-3-methyl-8-[6-[3-(1-oxidopiperidin-1-ium-1-yl)propoxy]-3-pyridyl]imidazo[4,5-c]quinolin-2-one.

In another embodiment, the compound according to formula (I) is a pharmaceutically acceptable salt of 7-fluoro-1-isopropyl-3-methyl-8-[6-[3-(1-oxidopiperidin-1-ium-1-yl)propoxy]-3-pyridyl]imidazo[4,5-c]quinolin-2-one.

According to other preferred embodiments of the ATM inhibitor used in the present disclosure, the ATM inhibitor is a compound represented by the following formula (II):

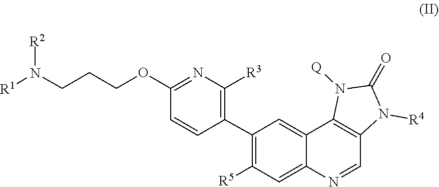

where:
Q is a cyclobutyl or cyclopentyl ring, each of which is optionally substituted by one hydroxy or methoxy group, or Q is an oxetanyl, tetrahydrofuranyl or oxanyl ring, each of which is optionally substituted by one methyl group;
R¹ is methyl;
R² is hydrogen or methyl; or R¹ and R² together form an azetidinyl, pyrrolidinyl or piperidinyl ring;
R³ is hydrogen or fluoro;
R⁴ is hydrogen or methyl; and
R⁵ is hydrogen or fluoro, or a pharmaceutically acceptable salt thereof.

The terms “cyclobutyl ring” and “cyclopentyl ring” refer to carbocyclic rings containing no heteroatoms. 1-methoxycyclobut-3-yl groups and 3-methoxycyclobut-1-yl groups have the same structure, as shown below.

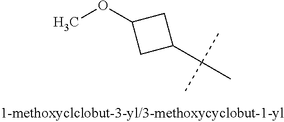

A cis-1-methoxy-cyclobut-3-yl group is equivalent to a cis-3-methoxy-cyclobut-1-yl and has the following structure:

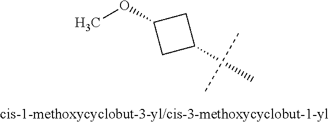

The same conventions apply to other cyclobutyl groups, for example 1-hydroxycyclobut-3-yl groups and 3-hydroxycyclobut-1-yl groups.

In a similar fashion, 1-methoxycyclopent-3-yl groups and 3-methoxycyclopent-1-yl groups have the same structure, as shown below.

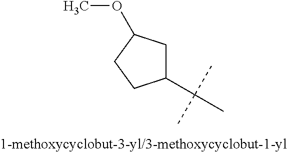

The term “oxetanyl ring” includes oxetan-2-yl and oxetan-3-yl groups, the structures of which are shown below.

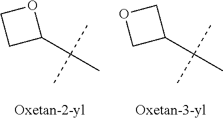

The term “tetrahydrofuranyl ring” includes tetrahydrofuran-2-yl and tetrahydrofuran-3-yl groups, the structures of which are shown below.

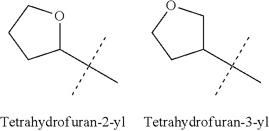

The term “oxanyl ring” includes oxan-2-yl, oxan-3-yl, and oxan-4-yl groups, the structures of which are shown below.

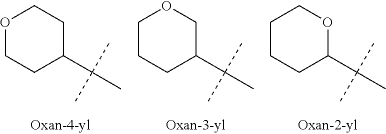

In the above structures the dashed line indicates the bonding position of the relevant group.

An oxanyl ring may also be referred to as a tetrahydropyranyl ring. Similarly, an oxan-4-yl ring may be referred to as a tetrahydropyran-4-yl ring; an oxan-3-yl ring may be referred to as a tetrahydropyran-3-yl ring, and an oxan-2-yl ring may be referred to as a tetrahydropyran-2-yl ring.

Where it is mentioned that “R¹ and R² together form an azetidinyl, pyrrolidinyl or piperidinyl ring”, this means the R¹ and R² groups are joined via a carbon-carbon covalent bond to form an unsubstituted alkylene chain of the appropriate length for the corresponding ring.

Some values of variable groups in formula (II) are as follows. Such values may be used in combination with any of the definitions, claims, or embodiments defined herein to provide further embodiments of compounds of formula (II):
a) Q is a cyclobutyl or cyclopentyl ring, each of which is substituted by one hydroxy or methoxy group, or Q is an oxetanyl, tetrahydrofuranyl or oxanyl ring, each of which is optionally substituted by one methyl group.
b) Q is a cyclobutyl ring substituted by one hydroxy or methoxy group, or Q is an oxetanyl or oxanyl ring, each of which is optionally substituted by one methyl group.
c) Q is a cyclobutyl ring substituted by one hydroxy or methoxy group, or Q is an oxetanyl or oxanyl ring.
d) Q is cyclobutyl, 1-methoxy-cyclobut-3-yl, 1-hydroxy-cyclobut-3-yl, 3-methoxycyclopent-1-yl, oxetan-3-yl, tetrahydrofuran-3-yl, oxan-3-yl, oxan-4-yl or 4-methyloxan-4-yl.
e) Q is 1-methoxy-cyclobut-3-yl, 1-hydroxy-cyclobut-3-yl, 3-methoxycyclopent-1-yl, oxetan-3-yl, oxan-3-yl, oxan-4-yl or 4-methyloxan-4-yl.
f) Q is 1-methoxy-cyclobut-3-yl, 1-hydroxy-cyclobut-3-yl or oxan-4-yl.
g) Q is cis-1-methoxy-cyclobut-3-yl or oxan-4-yl.
h) Q is a cyclobutyl or cyclopentyl ring, each of which is optionally substituted by one hydroxy or methoxy group.
i) Q is a cyclobutyl or cyclopentyl ring, each of which is optionally substituted by one methoxy group.
j) Q is a cyclobutyl ring substituted by one hydroxy or methoxy group.
k) Q is cyclobutyl, 1-hydroxy-cyclobut-3-yl or 1-methoxy-cyclobut-3-yl.
l) Q is cyclobutyl.
m) Q is a cyclopentyl ring substituted by one hydroxy or methoxy group.
n) Q is a cyclopentyl ring substituted by one methoxy group.
o) Q is 3-methoxycyclopent-1-yl.
p) Q is 1-hydroxy-cyclobut-3-yl or 1-methoxy-cyclobut-3-yl.
q) Q is cis-1-hydroxy-cyclobut-3-yl or cis-1-methoxy-cyclobut-3-yl.
r) Q is cis-1-methoxy-cyclobut-3-yl.
s) Q is an oxetanyl, tetrahydrofuranyl or oxanyl ring, each of which is optionally substituted by one methyl group.
t) Q is an oxetanyl or oxanyl ring, each of which is optionally substituted by one methyl group.
u) Q is an oxetanyl or tetrahydrofuranyl ring.
v) Q is an oxetanyl ring.
w) Q is oxetan-3-yl.
x) Q is a tetrahydrofuranyl ring.
y) Q is tetrahydrofuran-3-yl.
z) Q is an oxanyl ring optionally substituted by one methyl group.
aa) Q is an oxanyl ring.
bb) Q is oxan-4-yl.
cc) R¹ is methyl.
dd) R² is methyl.
ee) R² is hydrogen.
ff) R¹ is methyl and R² is hydrogen or methyl.
gg) R¹ and R² are both methyl; or R¹ and R² together form a pyrrolidinyl ring.
hh) R¹ and R² are both methyl.
ii) R¹ and R² together form an azetidinyl, pyrrolidinyl or piperidinyl ring.
jj) R¹ and R² together form an azetidinyl ring.
kk) R¹ and R² together form a pyrrolidinyl ring.
ll) R¹ and R² together form a piperidinyl ring.
mm) R³ and R⁵ are both hydrogen.
nn) R³ is hydrogen.
oo) R³ is fluoro.
pp) R⁴ is hydrogen.
qq) R⁴ is methyl.
rr) R⁵ is hydrogen.
ss) R⁵ is fluoro.

In one embodiment, the compound is represented by formula (II), or a pharmaceutically acceptable salt thereof, where:
Q is cyclobutyl, 1-methoxy-cyclobut-3-yl, 1-hydroxy-cyclobut-3-yl, 3-methoxycyclopent-1-yl, oxetan-3-yl, tetrahydrofuran-3-yl, oxan-3-yl, oxan-4-yl or 4-methyloxan-4-yl;
R¹ is methyl;
R² is hydrogen or methyl; or R¹ and R² together form an azetidinyl, pyrrolidinyl or piperidinyl ring;
R³ is hydrogen or fluoro;
R⁴ is hydrogen or methyl; and
R⁵ is hydrogen or fluoro.

In another embodiment, the compound is represented by formula (II), or a pharmaceutically acceptable salt thereof, where:
Q is 1-methoxy-cyclobut-3-yl, 1-hydroxy-cyclobut-3-yl, 3-methoxycyclopent-1-yl, oxetan-3-yl, oxan-3-yl, oxan-4-yl or 4-methyloxan-4-yl;
R¹ is methyl;
R² is hydrogen or methyl; or R¹ and R² together form an azetidinyl, pyrrolidinyl or piperidinyl ring;
R³ is hydrogen or fluoro;
R⁴ is hydrogen or methyl; and
R⁵ is hydrogen or fluoro.

In another embodiment, the compound is represented by formula (II), or a pharmaceutically acceptable salt thereof, where:
Q is 1-methoxy-cyclobut-3-yl, 1-hydroxy-cyclobut-3-yl or 3-methoxycyclopent-1-yl;
R¹ is methyl;
R² is hydrogen or methyl; or R¹ and R² together form an azetidinyl, pyrrolidinyl or piperidinyl ring;
R³ is hydrogen or fluoro;
R⁴ is hydrogen or methyl; and
R⁵ is hydrogen or fluoro.

In another embodiment, the compound is represented by formula (II), or a pharmaceutically acceptable salt thereof, where:
Q is oxetan-3-yl, oxan-3-yl, oxan-4-yl or 4-methyloxan-4-yl;
R¹ is methyl;
R² is hydrogen or methyl; or R¹ and R² together form an azetidinyl, pyrrolidinyl or piperidinyl ring;
R³ is hydrogen or fluoro;
R⁴ is hydrogen or methyl; and
R⁵ is hydrogen or fluoro.

In another embodiment, the compound is represented by formula (II), or a pharmaceutically acceptable salt thereof, where:
Q is cis-1-methoxy-cyclobut-3-yl or oxan-4-yl;
R¹ is methyl;
R² is methyl or hydrogen;
R³ is hydrogen;
R⁴ is methyl or hydrogen; and
R⁵ is hydrogen.

In another embodiment, the compound is represented by formula (II), or a pharmaceutically acceptable salt thereof, where the compound is selected from the group consisting of:
8-[6-(3-Dimethylaminopropoxy)pyridin-3-yl]-3-methyl-1-(oxan-4-yl)imidazo[5,4-c]quinolin-2-one;
8-[6-(3-Dimethylaminopropoxy)pyridin-3-yl]-1-(cis-3-methoxycyclobutyl)-3-methylimidazo[4,5-c]quinolin-2-one;
8-[6-(3-Dimethylaminopropoxy)pyridin-3-yl]-7-fluoro-3-methyl-1-(4-methyloxan-4-yl)imidazo[5,4-c]quinolin-2-one;
8-[6-(3-Dimethylaminopropoxy)pyridin-3-yl]-3-methyl-1-(oxetan-3-yl)imidazo[5,4-c]quinolin-2-one;
8-[6-(3-Dimethylaminopropoxy)pyridin-3-yl]-1-(cis-3-hydroxycyclobutyl)-3-methylimidazo[4,5-c]quinolin-2-one;
8-[6-(3-Dimethylaminopropoxy)pyridin-3-yl]-7-fluoro-1-(cis-3-methoxycyclobutyl)-3-methylimidazo[4,5-c]quinolin-2-one;
8-[6-(3-Dimethylaminopropoxy)pyridin-3-yl]-3-methyl-1-(4-methyloxan-4-yl)imidazo[5,4-c]quinolin-2-one;
8-[6-(3-Dimethylaminopropoxy)pyridin-3-yl]-7-fluoro-3-methyl-1-(oxan-4-yl)imidazo[5,4-c]quinolin-2-one;
8-[6-(3-Dimethylaminopropoxy)pyridin-3-yl]-7-fluoro-3-methyl-1-(oxetan-3-yl)imidazo[5,4-c]quinolin-2-one;
8-[6-(3-Dimethylaminopropoxy)pyridin-3-yl]-7-fluoro-3-methyl-1-[(3R)-oxan-3-yl]imidazo[5,4-c]quinolin-2-one;
8-[6-(3-Dimethylaminopropoxy)pyridin-3-yl]-7-fluoro-3-methyl-1-[(3S)-oxan-3-yl]imidazo[5,4-c]quinolin-2-one;
8-[6-(3-Dimethylaminopropoxy)pyridin-3-yl]-1-(cis-3-methoxycyclobutyl)-3H-imidazo[4,5-c]quinolin-2-one;
8-[6-(3-Dimethylaminopropoxy)pyridin-3-yl]-1-(oxan-4-yl)-3H-imidazo[4,5-c]quinolin-2-one;
8-[6-[3-(Azetidin-1-yl)propoxy]pyridin-3-yl]-1-(cis-3-hydroxycyclobutyl)-3-methylimidazo[4,5-c]quinolin-2-one;
1-(3-cis-Hydroxycyclobutyl)-3-methyl-8-[6-(3-pyrrolidin-1-ylpropoxy)pyridin-3-yl]imidazo[4,5-c]quinolin-2-one;
8-[6-(3-Dimethylaminopropoxy)pyridin-3-yl]-3-methyl-1-[(3R)-oxan-3-yl]imidazo[5,4-c]quinolin-2-one;
8-[6-(3-Dimethylaminopropoxy)pyridin-3-yl]-3-methyl-1-[(3S)-oxan-3-yl]imidazo[5,4-c]quinolin-2-one;
8-[6-(3-Dimethylaminopropoxy)-2-fluoropyridin-3-yl]-1-(cis-3-methoxycyclobutyl)-3-methylimidazo[4,5-c]quinolin-2-one;
8-[6-(3-Dimethylaminopropoxy)-2-fluoropyridin-3-yl]-7-fluoro-1-(cis-3-methoxycyclobutyl)-3-methylimidazo[4,5-c]quinolin-2-one;
8-[6-(3-Dimethylaminopropoxy)-2-fluoropyridin-3-yl]-3-methyl-1-[(3S)-oxan-3-yl]imidazo[5,4-c]quinolin-2-one;
8-[6-(3-Dimethylaminopropoxy)-2-fluoropyridin-3-yl]-3-methyl-1-(oxan-4-yl)imidazo[5,4-c]quinolin-2-one;
8-[6-(3-Dimethylaminopropoxy)-2-fluoropyridin-3-yl]-3-methyl-1-[(3R)-oxan-3-yl]imidazo[5,4-c]quinolin-2-one;
7-Fluoro-3-methyl-8-[6-(3-pyrrolidin-1-ylpropoxy)-3-pyridyl]-1-[(3S)-tetrahydropyran-3-yl]imidazo[4,5-c]quinolin-2-one;
7-Fluoro-3-methyl-8-[6-(3-pyrrolidin-1-ylpropoxy)-3-pyridyl]-1-[(3S)-tetrahydropyran-3-yl]imidazo[4,5-c]quinolin-2-one;
8-[6-(3-Dimethylaminopropoxy)pyridin-3-yl]-1-[(1R,3R)-3-methoxycyclopentyl]-3-methylimidazo[4,5-c]quinolin-2-one;
8-[6-(3-Dimethylaminopropoxy)pyridin-3-yl]-1-[(1S,3S)-3-methoxycyclopentyl]-3-methylimidazo[4,5-c]quinolin-2-one;
7-Fluoro-1-(cis-3-methoxycyclobutyl)-3-methyl-8-[6-(3-pyrrolidin-1-ylpropoxy)pyridin-3-yl]imidazo[4,5-c]quinolin-2-one;
1-(cis-3-Methoxycyclobutyl)-3-methyl-8-[6-(3-pyrrolidin-1-ylpropoxy)pyridin-3-yl]imidazo[4,5-c]quinolin-2-one;
3-Methyl-1-[(3S)-oxan-3-yl]-8-[6-(3-pyrrolidin-1-ylpropoxy)pyridin-3-yl]imidazo[5,4-c]quinolin-2-one;
3-Methyl-1-(oxan-4-yl)-8-[6-(3-pyrrolidin-1-ylpropoxy)pyridin-3-yl]imidazo[5,4-c]quinolin-2-one;
3-Methyl-1-[(3S)-oxan-3-yl]-8-[6-(3-piperidin-1-ylpropoxy)pyridin-3-yl]imidazo[5,4-c]quinolin-2-one;
3-Methyl-1-[(3R)-oxan-3-yl]-8-[6-(3-piperidin-1-ylpropoxy)pyridin-3-yl]imidazo[5,4-c]quinolin-2-one;
1-(cis-3-Methoxycyclobutyl)-3-methyl-8-[6-(3-piperidin-1-ylpropoxy)pyridin-3-yl]imidazo[4,5-c]quinolin-2-one;
8-[6-[3-(Azetidin-1-yl)propoxy]pyridin-3-yl]-3-methyl-1-[(3R)-oxan-3-yl]imidazo[5,4-c]quinolin-2-one;
1-(cis-3-Methoxycyclobutyl)-8-[6-(3-pyrrolidin-1-ylpropoxy)pyridin-3-yl]-3H-imidazo[4,5-c]quinolin-2-one;
1-(Oxan-4-yl)-8-[6-(3-piperidin-1-ylpropoxy)pyridin-3-yl]-3H-imidazo[4,5-c]quinolin-2-one;
3-Methyl-1-(oxan-4-yl)-8-[6-(3-piperidin-1-ylpropoxy)pyridin-3-yl]imidazo[5,4-c]quinolin-2-one;
8-[6-[3-(Azetidin-1-yl)propoxy]pyridin-3-yl]-1-(cis-3-methoxycyclobutyl)-3-methylimidazo[4,5-c]quinolin-2-one;
8-[6-[3-(Azetidin-1-yl)propoxy]pyridin-3-yl]-3-methyl-1-(oxan-4-yl)imidazo[5,4-c]quinolin-2-one;
3-Methyl-8-[6-(3-methylaminopropoxy)pyridin-3-yl]-1-(oxan-4-yl)imidazo[5,4-c]quinolin-2-one;
3-Methyl-8-[6-(3-methylaminopropoxy)pyridin-3-yl]-1-[(3S)-oxan-3-yl]imidazo[5,4-c]quinolin-2-one; and
1-(cis-3-Methoxycyclobutyl)-3-methyl-8-[6-(3-methylaminopropoxy)pyridin-3-yl]imidazo[4,5-c]quinolin-2-one;
3-Methyl-8-[6-[3-(methylamino)propoxy]-3-pyridyl]-1-[(3R)-tetrahydropyran-3-yl]imidazo[4,5-c]quinolin-2-one;
8-[6-[3-(Dimethylamino)propoxy]-3-pyridyl]-3-methyl-1-[(3R)-tetrahydrofuran-3-yl]imidazo[4,5-c]quinolin-2-one;
8-[6-[3-(Dimethylamino)propoxy]-3-pyridyl]-3-methyl-1-[(3S)-tetrahydrofuran-3-yl]imidazo[4,5-c]quinolin-2-one;
1-Cyclobutyl-8-[6-[3-(dimethylamino)propoxy]-3-pyridyl]-3-methyl-imidazo[4,5-c]quinolin-2-one;
7-Fluoro-3-methyl-8-[6-[3-(1-piperidyl)propoxy]-3-pyridyl]-1-[(3S)-tetrahydropyran-3-yl]imidazo[4,5-c]quinolin-2-one;
8-[6-[3-(Dimethylamino)propoxy]-2-fluoro-3-pyridyl]-7-fluoro-3-methyl-1-[(3S)-tetrahydropyran-3-yl]imidazo[4,5-c]quinolin-2-one;
8-[6-[3-(Dimethylamino)propoxy]-2-fluoro-3-pyridyl]-3-methyl-1-[(3S)-tetrahydrofuran-3-yl]imidazo[4,5-c]quinolin-2-one;
8-[6-[3-(Dimethylamino)propoxy]-2-fluoro-3-pyridyl]-3-methyl-1-[(3R)-tetrahydrofuran-3-yl]imidazo[4,5-c]quinolin-2-one;
1-Cyclobutyl-8-[6-[3-(dimethylamino)propoxy]-2-fluoro-3-pyridyl]-3-methyl-imidazo[4,5-c]quinolin-2-one;
8-[6-[3-(Dimethylamino)propoxy]-2-fluoro-3-pyridyl]-3-methyl-1-(oxetan-3-yl)imidazo[4,5-c]quinolin-2-one;
7-Fluoro-3-methyl-8-[6-[3-(1-piperidyl)propoxy]-3-pyridyl]-1-tetrahydropyran-4-yl-imidazo[4,5-c]quinolin-2-one
3-Methyl-8-[6-[3-(1-piperidyl)propoxy]-3-pyridyl]-1-[(3R)-tetrahydrofuran-3-yl]imidazo[4,5-c]quinolin-2-one;
3-Methyl-8-[6-[3-(1-piperidyl)propoxy]-3-pyridyl]-1-[(3S)-tetrahydrofuran-3-yl]imidazo[4,5-c]quinolin-2-one;
3-Methyl-1-(oxetan-3-yl)-8-[6-[3-(1-piperidyl)propoxy]-3-pyridyl]imidazo[4,5-c]quinolin-2-one;
1-Cyclobutyl-3-methyl-8-[6-[3-(1-piperidyl)propoxy]-3-pyridyl]imidazo[4,5-c]quinolin-2-one;
1-Cyclobutyl-3-methyl-8-[6-(3-pyrrolidin-1-ylpropoxy)-3-pyridyl]imidazo[4,5-c]quinolin-2-one;
3-Methyl-8-[6-(3-pyrrolidin-1-ylpropoxy)-3-pyridyl]-1-[(3R)-tetrahydropyran-3-yl]imidazo[4,5-c]quinolin-2-one;
8-[6-[3-(Azetidin-1-yl)propoxy]-3-pyridyl]-3-methyl-1-[(3S)-tetrahydropyran-3-yl]imidazo[4,5-c]quinolin-2-one;
8-[2-Fluoro-6-(3-pyrrolidin-1-ylpropoxy)-3-pyridyl]-3-methyl-1-[(3S)-tetrahydropyran-3-yl]imidazo[4,5-c]quinolin-2-one;
8-[6-[3-(Dimethylamino)propoxy]-3-pyridyl]-7-fluoro-1-[(1R,3R)-3-methoxycyclopentyl]-3-methyl-imidazo[4,5-c]quinolin-2-one;
8-[6-[3-(Dimethylamino)propoxy]-3-pyridyl]-7-fluoro-1-[(1S,3S)-3-methoxycyclopentyl]-3-methyl-imidazo[4,5-c]quinolin-2-one;
1-[(1R,3R)-3-Methoxycyclopentyl]-3-methyl-8-[6-[3-(1-piperidyl)propoxy]-3-pyridyl]imidazo[4,5-c]quinolin-2-one;
1-[(1S,3S)-3-Methoxycyclopentyl]-3-methyl-8-[6-[3-(1-piperidyl)propoxy]-3-pyridyl]imidazo[4,5-c]quinolin-2-one;
1-[(1S,3S)-3-Methoxycyclopentyl]-3-methyl-8-[6-(3-pyrrolidin-1-ylpropoxy)-3-pyridyl]imidazo[4,5-c]quinolin-2-one;
1-[(1R,3R)-3-Methoxycyclopentyl]-3-methyl-8-[6-(3-pyrrolidin-1-ylpropoxy)-3-pyridyl]imidazo[4,5-c]quinolin-2-one;
8-[6-[3-(Dimethylamino)propoxy]-2-fluoro-3-pyridyl]-1-[(1R,3R)-3-methoxycyclopentyl]-3-methyl-imidazo[4,5-c]quinolin-2-one;
8-[6-[3-(Dimethylamino)propoxy]-2-fluoro-3-pyridyl]-1-[(1S,3S)-3-methoxycyclopentyl]-3-methyl-imidazo[4,5-c]quinolin-2-one;
8-[6-[3-(Dimethylamino)propoxy]-3-pyridyl]-7-fluoro-1-[(1R,3S)-3-methoxycyclopentyl]-3-methyl-imidazo[4,5-c]quinolin-2-one; and
8-[6-[3-(Dimethylamino)propoxy]-3-pyridyl]-7-fluoro-1-[(1S,3R)-3-methoxycyclopentyl]-3-methyl-imidazo[4,5-c]quinolin-2-one.

In another embodiment, the compound is represented by formula (II), or a pharmaceutically acceptable salt thereof, where the compound is selected from the group consisting of:
8-[6-(3-Dimethylaminopropoxy)pyridin-3-yl]-3-methyl-1-(oxan-4-yl)imidazo[5,4-c]quinolin-2-one;
8-[6-(3-Dimethylaminopropoxy)pyridin-3-yl]-1-(cis-3-methoxycyclobutyl)-3-methylimidazo[4,5-c]quinolin-2-one;
8-[6-(3-Dimethylaminopropoxy)pyridin-3-yl]-7-fluoro-3-methyl-1-(4-methyloxan-4-yl)imidazo[5,4-c]quinolin-2-one;
8-[6-(3-Dimethylaminopropoxy)pyridin-3-yl]-3-methyl-1-(oxetan-3-yl)imidazo[5,4-c]quinolin-2-one;
8-[6-(3-Dimethylaminopropoxy)pyridin-3-yl]-1-(cis-3-hydroxycyclobutyl)-3-methylimidazo[4,5-c]quinolin-2-one;
8-[6-(3-Dimethylaminopropoxy)pyridin-3-yl]-7-fluoro-1-(cis-3-methoxycyclobutyl)-3-methylimidazo[4,5-c]quinolin-2-one;
8-[6-(3-Dimethylaminopropoxy)pyridin-3-yl]-3-methyl-1-(4-methyloxan-4-yl)imidazo[5,4-c]quinolin-2-one;
8-[6-(3-Dimethylaminopropoxy)pyridin-3-yl]-7-fluoro-3-methyl-1-(oxan-4-yl)imidazo[5,4-c]quinolin-2-one;
8-[6-(3-Dimethylaminopropoxy)pyridin-3-yl]-7-fluoro-3-methyl-1-(oxetan-3-yl)imidazo[5,4-c]quinolin-2-one;
8-[6-(3-Dimethylaminopropoxy)pyridin-3-yl]-7-fluoro-3-methyl-1-[(3R)-oxan-3-yl]imidazo[5,4-c]quinolin-2-one;
8-[6-(3-Dimethylaminopropoxy)pyridin-3-yl]-7-fluoro-3-methyl-1-[(3S)-oxan-3-yl]imidazo[5,4-c]quinolin-2-one;
8-[6-(3-Dimethylaminopropoxy)pyridin-3-yl]-1-(cis-3-methoxycyclobutyl)-3H-imidazo[4,5-c]quinolin-2-one;
8-[6-(3-Dimethylaminopropoxy)pyridin-3-yl]-1-(oxan-4-yl)-3H-imidazo[4,5-c]quinolin-2-one;
8-[6-[3-(Azetidin-1-yl)propoxy]pyridin-3-yl]-1-(cis-3-hydroxycyclobutyl)-3-methylimidazo[4,5-c]quinolin-2-one;
1-(3-cis-Hydroxycyclobutyl)-3-methyl-8-[6-(3-pyrrolidin-1-ylpropoxy)pyridin-3-yl]imidazo[4,5-c]quinolin-2-one;
8-[6-(3-Dimethylaminopropoxy)pyridin-3-yl]-3-methyl-1-[(3R)-oxan-3-yl]imidazo[5,4-c]quinolin-2-one;
8-[6-(3-Dimethylaminopropoxy)pyridin-3-yl]-3-methyl-1-[(3S)-oxan-3-yl]imidazo[5,4-c]quinolin-2-one;
8-[6-(3-Dimethylaminopropoxy)-2-fluoropyridin-3-yl]-1-(cis-3-methoxycyclobutyl)-3-methylimidazo[4,5-c]quinolin-2-one;
8-[6-(3-Dimethylaminopropoxy)-2-fluoropyridin-3-yl]-7-fluoro-1-(cis-3-methoxycyclobutyl)-3-methylimidazo[4,5-c]quinolin-2-one;
8-[6-(3-Dimethylaminopropoxy)-2-fluoropyridin-3-yl]-3-methyl-1-[(3S)-oxan-3-yl]imidazo[5,4-c]quinolin-2-one;
8-[6-(3-Dimethylaminopropoxy)-2-fluoropyridin-3-yl]-3-methyl-1-(oxan-4-yl)imidazo[5,4-c]quinolin-2-one;
8-[6-(3-Dimethylaminopropoxy)-2-fluoropyridin-3-yl]-3-methyl-1-[(3R)-oxan-3-yl]imidazo[5,4-c]quinolin-2-one;
3-Methyl-1-[(3R)-oxan-3-yl]-8-[6-(3-pyrrolidin-1-ylpropoxy)pyridin-3-yl]imidazo[5,4-c]quinolin-2-one;
8-[6-(3-Dimethylaminopropoxy)pyridin-3-yl]-1-[(1R,3R)-3-methoxycyclopentyl]-3-methylimidazo[4,5-c]quinolin-2-one;
8-[6-(3-Dimethylaminopropoxy)pyridin-3-yl]-1-[(1S,3S)-3-methoxycyclopentyl]-3-methylimidazo[4,5-c]quinolin-2-one;
7-Fluoro-1-(cis-3-methoxycyclobutyl)-3-methyl-8-[6-(3-pyrrolidin-1-ylpropoxy)pyridin-3-yl]imidazo[4,5-c]quinolin-2-one;
1-(cis-3-Methoxycyclobutyl)-3-methyl-8-[6-(3-pyrrolidin-1-ylpropoxy)pyridin-3-yl]imidazo[4,5-c]quinolin-2-one;
3-Methyl-1-[(3S)-oxan-3-yl]-8-[6-(3-pyrrolidin-1-ylpropoxy)pyridin-3-yl]imidazo[5,4-c]quinolin-2-one;
3-Methyl-1-(oxan-4-yl)-8-[6-(3-pyrrolidin-1-ylpropoxy)pyridin-3-yl]imidazo[5,4-c]quinolin-2-one;
3-Methyl-1-[(3S)-oxan-3-yl]-8-[6-(3-piperidin-1-ylpropoxy)pyridin-3-yl]imidazo[5,4-c]quinolin-2-one;
3-Methyl-1-[(3R)-oxan-3-yl]-8-[6-(3-piperidin-1-ylpropoxy)pyridin-3-yl]imidazo[5,4-c]quinolin-2-one;
1-(cis-3-Methoxycyclobutyl)-3-methyl-8-[6-(3-piperidin-1-ylpropoxy)pyridin-3-yl]imidazo[4,5-c]quinolin-2-one;
8-[6-[3-(Azetidin-1-yl)propoxy]pyridin-3-yl]-3-methyl-1-[(3R)-oxan-3-yl]imidazo[5,4-c]quinolin-2-one;
1-(cis-3-Methoxycyclobutyl)-8-[6-(3-pyrrolidin-1-ylpropoxy)pyridin-3-yl]-3H-imidazo[4,5-c]quinolin-2-one;
1-(Oxan-4-yl)-8-[6-(3-piperidin-1-ylpropoxy)pyridin-3-yl]-3H-imidazo[4,5-c]quinolin-2-one;
3-Methyl-1-(oxan-4-yl)-8-[6-(3-piperidin-1-ylpropoxy)pyridin-3-yl]imidazo[5,4-c]quinolin-2-one;
8-[6-[3-(Azetidin-1-yl)propoxy]pyridin-3-yl]-1-(cis-3-methoxycyclobutyl)-3-methylimidazo[4,5-c]quinolin-2-one;
8-[6-[3-(Azetidin-1-yl)propoxy]pyridin-3-yl]-3-methyl-1-(oxan-4-yl)imidazo[5,4-c]quinolin-2-one;
3-Methyl-8-[6-(3-methylaminopropoxy)pyridin-3-yl]-1-(oxan-4-yl)imidazo[5,4-c]quinolin-2-one;
3-Methyl-8-[6-(3-methylaminopropoxy)pyridin-3-yl]-1-[(3S)-oxan-3-yl]imidazo[5,4-c]quinolin-2-one; and
1-(cis-3-Methoxycyclobutyl)-3-methyl-8-[6-(3-methylaminopropoxy)pyridin-3-yl]imidazo[4,5-c]quinolin-2-one.

In another embodiment, the compound is represented by formula (II), or a pharmaceutically acceptable salt thereof, where the compound is selected from the group consisting of:
8-[6-(3-Dimethylaminopropoxy)pyridin-3-yl]-3-methyl-1-(oxan-4-yl)imidazo[5,4-c]quinolin-2-one;
8-[6-(3-Dimethylaminopropoxy)pyridin-3-yl]-1-(cis-3-methoxycyclobutyl)-3-methylimidazo[4,5-c]quinolin-2-one;
8-[6-(3-Dimethylaminopropoxy)pyridin-3-yl]-7-fluoro-3-methyl-1-(4-methyloxan-4-yl)imidazo[5,4-c]quinolin-2-one;
8-[6-(3-Dimethylaminopropoxy)pyridin-3-yl]-3-methyl-1-(oxetan-3-yl)imidazo[5,4-c]quinolin-2-one;
8-[6-(3-Dimethylaminopropoxy)pyridin-3-yl]-1-(cis-3-hydroxycyclobutyl)-3-methylimidazo[4,5-c]quinolin-2-one;
8-[6-(3-Dimethylaminopropoxy)pyridin-3-yl]-7-fluoro-1-(cis-3-methoxycyclobutyl)-3-methylimidazo[4,5-c]quinolin-2-one;
8-[6-(3-Dimethylaminopropoxy)pyridin-3-yl]-3-methyl-1-(4-methyloxan-4-yl)imidazo[5,4-c]quinolin-2-one;
8-[6-(3-Dimethylaminopropoxy)pyridin-3-yl]-7-fluoro-3-methyl-1-(oxan-4-yl)imidazo[5,4-c]quinolin-2-one;
8-[6-(3-Dimethylaminopropoxy)pyridin-3-yl]-7-fluoro-3-methyl-1-(oxetan-3-yl)imidazo[5,4-c]quinolin-2-one;
8-[6-(3-Dimethylaminopropoxy)pyridin-3-yl]-7-fluoro-3-methyl-1-[(3R)-oxan-3-yl]imidazo[5,4-c]quinolin-2-one;
8-[6-(3-Dimethylaminopropoxy)pyridin-3-yl]-7-fluoro-3-methyl-1-[(3S)-oxan-3-yl]imidazo[5,4-c]quinolin-2-one;
8-[6-(3-Dimethylaminopropoxy)pyridin-3-yl]-1-(cis-3-methoxycyclobutyl)-3H-imidazo[4,5-c]quinolin-2-one;
8-[6-(3-Dimethylaminopropoxy)pyridin-3-yl]-1-(oxan-4-yl)-3H-imidazo[4,5-c]quinolin-2-one;
8-[6-[3-(Azetidin-1-yl)propoxy]pyridin-3-yl]-1-(cis-3-hydroxycyclobutyl)-3-methylimidazo[4,5-c]quinolin-2-one;
1-(3-cis-Hydroxycyclobutyl)-3-methyl-8-[6-(3-pyrrolidin-1-ylpropoxy)pyridin-3-yl]imidazo[4,5-c]quinolin-2-one;
8-[6-(3-Dimethylaminopropoxy)pyridin-3-yl]-3-methyl-1-[(3R)-oxan-3-yl]imidazo[5,4-c]quinolin-2-one;
8-[6-(3-Dimethylaminopropoxy)pyridin-3-yl]-3-methyl-1-[(3S)-oxan-3-yl]imidazo[5,4-c]quinolin-2-one;
8-[6-(3-Dimethylaminopropoxy)-2-fluoropyridin-3-yl]-1-(cis-3-methoxycyclobutyl)-3-methylimidazo[4,5-c]quinolin-2-one;
8-[6-(3-Dimethylaminopropoxy)-2-fluoropyridin-3-yl]-7-fluoro-1-(cis-3-methoxycyclobutyl)-3-methylimidazo[4,5-c]quinolin-2-one;
8-[6-(3-Dimethylaminopropoxy)-2-fluoropyridin-3-yl]-3-methyl-1-[(3S)-oxan-3-yl]imidazo[5,4-c]quinolin-2-one;
8-[6-(3-Dimethylaminopropoxy)-2-fluoropyridin-3-yl]-3-methyl-1-(oxan-4-yl)imidazo[5,4-c]quinolin-2-one;
8-[6-(3-Dimethylaminopropoxy)-2-fluoropyridin-3-yl]-3-methyl-1-[(3R)-oxan-3-yl]imidazo[5,4-c]quinolin-2-one;
3-Methyl-1-[(3R)-oxan-3-yl]-8-[6-(3-pyrrolidin-1-ylpropoxy)pyridin-3-yl]imidazo[5,4-c]quinolin-2-one;
7-Fluoro-1-(cis-3-methoxycyclobutyl)-3-methyl-8-[6-(3-pyrrolidin-1-ylpropoxy)pyridin-3-yl]imidazo[4,5-c]quinolin-2-one;
1-(cis-3-Methoxycyclobutyl)-3-methyl-8-[6-(3-pyrrolidin-1-ylpropoxy)pyridin-3-yl]imidazo[4,5-c]quinolin-2-one;
3-Methyl-1-[(3S)-oxan-3-yl]-8-[6-(3-pyrrolidin-1-ylpropoxy)pyridin-3-yl]imidazo[5,4-c]quinolin-2-one;
3-Methyl-1-(oxan-4-yl)-8-[6-(3-pyrrolidin-1-ylpropoxy)pyridin-3-yl]imidazo[5,4-c]quinolin-2-one;
3-Methyl-1-[(3S)-oxan-3-yl]-8-[6-(3-piperidin-1-ylpropoxy)pyridin-3-yl]imidazo[5,4-c]quinolin-2-one;
3-Methyl-1-[(3R)-oxan-3-yl]-8-[6-(3-piperidin-1-ylpropoxy)pyridin-3-yl]imidazo[5,4-c]quinolin-2-one;
1-(cis-3-Methoxycyclobutyl)-3-methyl-8-[6-(3-piperidin-1-ylpropoxy)pyridin-3-yl]imidazo[4,5-c]quinolin-2-one;
8-[6-[3-(Azetidin-1-yl)propoxy]pyridin-3-yl]-3-methyl-1-[(3R)-oxan-3-yl]imidazo[5,4-c]quinolin-2-one;
1-(cis-3-Methoxycyclobutyl)-8-[6-(3-pyrrolidin-1-ylpropoxy)pyridin-3-yl]-3H-imidazo[4,5-c]quinolin-2-one;
1-(Oxan-4-yl)-8-[6-(3-piperidin-1-ylpropoxy)pyridin-3-yl]-3H-imidazo[4,5-c]quinolin-2-one;
3-Methyl-1-(oxan-4-yl)-8-[6-(3-piperidin-1-ylpropoxy)pyridin-3-yl]imidazo[5,4-c]quinolin-2-one;
8-[6-[3-(Azetidin-1-yl)propoxy]pyridin-3-yl]-1-(cis-3-methoxycyclobutyl)-3-methylimidazo[4,5-c]quinolin-2-one;
8-[6-[3-(Azetidin-1-yl)propoxy]pyridin-3-yl]-3-methyl-1-(oxan-4-yl)imidazo[5,4-c]quinolin-2-one;
3-Methyl-8-[6-(3-methylaminopropoxy)pyridin-3-yl]-1-(oxan-4-yl)imidazo[5,4-c]quinolin-2-one;
3-Methyl-8-[6-(3-methylaminopropoxy)pyridin-3-yl]-1-[(3S)-oxan-3-yl]imidazo[5,4-c]quinolin-2-one; and
1-(cis-3-Methoxycyclobutyl)-3-methyl-8-[6-(3-methylaminopropoxy)pyridin-3-yl]imidazo[4,5-c]quinolin-2-one.

In another embodiment, the compound is represented by formula (II), or a pharmaceutically acceptable salt thereof, where the compound is selected from the group consisting of:
7-Fluoro-3-methyl-8-[6-(3-pyrrolidin-1-ylpropoxy)-3-pyridyl]-1-[(3S)-tetrahydropyran-3-yl]imidazo[4,5-c]quinolin-2-one;
7-Fluoro-3-methyl-8-[6-(3-pyrrolidin-1-ylpropoxy)-3-pyridyl]-1-[(3R)-tetrahydropyran-3-yl]imidazo[4,5-c]quinolin-2-one;
3-Methyl-8-[6-[3-(methylamino)propoxy]-3-pyridyl]-1-[(3R)-tetrahydropyran-3-yl]imidazo[4,5-c]quinolin-2-one;
8-[6-[3-(Dimethylamino)propoxy]-3-pyridyl]-3-methyl-1-[(3R)-tetrahydrofuran-3-yl]imidazo[4,5-c]quinolin-2-one;
8-[6-[3-(Dimethylamino)propoxy]-3-pyridyl]-3-methyl-1-[(3S)-tetrahydrofuran-3-yl]imidazo[4,5-c]quinolin-2-one;
1-Cyclobutyl-8-[6-[3-(dimethylamino)propoxy]-3-pyridyl]-3-methyl-imidazo[4,5-c]quinolin-2-one;
7-Fluoro-3-methyl-8-[6-[3-(1-piperidyl)propoxy]-3-pyridyl]-1-[(3S)-tetrahydropyran-3-yl]imidazo[4,5-c]quinolin-2-one;
8-[6-[3-(Dimethylamino)propoxy]-2-fluoro-3-pyridyl]-7-fluoro-3-methyl-1-[(3S)-tetrahydropyran-3-yl]imidazo[4,5-c]quinolin-2-one;
8-[6-[3-(Dimethylamino)propoxy]-2-fluoro-3-pyridyl]-3-methyl-1-[(3S)-tetrahydrofuran-3-yl]imidazo[4,5-c]quinolin-2-one;
8-[6-[3-(Dimethylamino)propoxy]-2-fluoro-3-pyridyl]-3-methyl-1-[(3R)-tetrahydrofuran-3-yl]imidazo[4,5-c]quinolin-2-one;
1-Cyclobutyl-8-[6-[3-(dimethylamino)propoxy]-2-fluoro-3-pyridyl]-3-methyl-imidazo[4,5-c]quinolin-2-one;
8-[6-[3-(Dimethylamino)propoxy]-2-fluoro-3-pyridyl]-3-methyl-1-(oxetan-3-yl)imidazo[4,5-c]quinolin-2-one;
7-Fluoro-3-methyl-8-[6-[3-(1-piperidyl)propoxy]-3-pyridyl]-1-tetrahydropyran-4-yl-imidazo[4,5-c]quinolin-2-one
13-Methyl-8-[6-[3-(1-piperidyl)propoxy]-3-pyridyl]-1-[(3R)-tetrahydrofuran-3-yl]imidazo[4,5-c]quinolin-2-one;
3-Methyl-8-[6-[3-(1-piperidyl)propoxy]-3-pyridyl]-1-[(3S)-tetrahydrofuran-3-yl]imidazo[4,5-c]quinolin-2-one;
3-Methyl-1-(oxetan-3-yl)-8-[6-[3-(1-piperidyl)propoxy]-3-pyridyl]imidazo[4,5-c]quinolin-2-one;
1-Cyclobutyl-3-methyl-8-[6-[3-(1-piperidyl)propoxy]-3-pyridyl]imidazo[4,5-c]quinolin-2-one;
1-Cyclobutyl-3-methyl-8-[6-(3-pyrrolidin-1-ylpropoxy)-3-pyridyl]imidazo[4,5-c]quinolin-2-one;
3-Methyl-8-[6-(3-pyrrolidin-1-ylpropoxy)-3-pyridyl]-1-[(3R)-tetrahydropyran-3-yl]imidazo[4,5-c]quinolin-2-one;
8-[6-[3-(Azetidin-1-yl)propoxy]-3-pyridyl]-3-methyl-1-[(3S)-tetrahydropyran-3-yl]imidazo[4,5-c]quinolin-2-one;
8-[2-Fluoro-6-(3-pyrrolidin-1-ylpropoxy)-3-pyridyl]-3-methyl-1-[(3S)-tetrahydropyran-3-yl]imidazo[4,5-c]quinolin-2-one;
8-[6-[3-(Dimethylamino)propoxy]-3-pyridyl]-7-fluoro-1-[(1R,3R)-3-methoxycyclopentyl]-3-methyl-imidazo[4,5-c]quinolin-2-one;
8-[6-[3-(Dimethylamino)propoxy]-3-pyridyl]-7-fluoro-1-[(1S,3S)-3-methoxycyclopentyl]-3-methyl-imidazo[4,5-c]quinolin-2-one;
1-[(1R,3R)-3-Methoxycyclopentyl]-3-methyl-8-[6-[3-(1-piperidyl)propoxy]-3-pyridyl]imidazo[4,5-c]quinolin-2-one;
1-[(1S,3S)-3-Methoxycyclopentyl]-3-methyl-8-[6-[3-(1-piperidyl)propoxy]-3-pyridyl]imidazo[4,5-c]quinolin-2-one;
1-[(1S,3S)-3-Methoxycyclopentyl]-3-methyl-8-[6-(3-pyrrolidin-1-ylpropoxy)-3-pyridyl]imidazo[4,5-c]quinolin-2-one;
1-[(1R,3R)-3-Methoxycyclopentyl]-3-methyl-8-[6-(3-pyrrolidin-1-ylpropoxy)-3-pyridyl]imidazo[4,5-c]quinolin-2-one;
8-[6-[3-(Dimethylamino)propoxy]-2-fluoro-3-pyridyl]-1-[(1R,3R)-3-methoxycyclopentyl]-3-methyl-imidazo[4,5-c]quinolin-2-one;
8-[6-[3-(Dimethylamino)propoxy]-2-fluoro-3-pyridyl]-1-[(1S,3S)-3-methoxycyclopentyl]-3-methyl-imidazo[4,5-c]quinolin-2-one;
8-[6-[3-(Dimethylamino)propoxy]-3-pyridyl]-7-fluoro-1-[(1R,3S)-3-methoxycyclopentyl]-3-methyl-imidazo[4,5-c]quinolin-2-one; and
8-[6-[3-(Dimethylamino)propoxy]-3-pyridyl]-7-fluoro-1-[(1S,3R)-3-methoxycyclopentyl]-3-methyl-imidazo[4,5-c]quinolin-2-one.

In another embodiment, the compound is represented by formula (II), or a pharmaceutically acceptable salt thereof, where the compound is selected from the group consisting of:
8-[6-(3-Dimethylaminopropoxy)pyridin-3-yl]-1-[(1R,3R)-3-methoxycyclopentyl]-3-methylimidazo[4,5-c]quinolin-2-one; and
8-[6-(3-Dimethylaminopropoxy)pyridin-3-yl]-1-[(1S,3S)-3-methoxycyclopentyl]-3-methylimidazo[4,5-c]quinolin-2-one.
   In one embodiment there is provided 8-[6-(3-dimethylaminopropoxy)pyridin-3-yl]-1-(cis-3-methoxycyclobutyl)-3-methylimidazo[4,5-c]quinolin-2-one, or a pharmaceutically acceptable salt thereof.
   In one embodiment there is provided 8-[6-(3-dimethylaminopropoxy)pyridin-3-yl]-1-(cis-3-methoxycyclobutyl)-3-methylimidazo[4,5-c]quinolin-2-one.
   In one embodiment there is provided a pharmaceutically acceptable salt of 8-[6-(3-dimethylaminopropoxy)pyridin-3-yl]-1-(cis-3-methoxycyclobutyl)-3-methylimidazo[4,5-c]quinolin-2-one.

In another embodiment, the compound represented by formula (II) is 8-[6-(3-dimethylaminopropoxy)pyridin-3-yl]-3-methyl-1-(oxan-4-yl)imidazo[5,4-c]quinolin-2-one, or a pharmaceutically acceptable salt thereof.

In another embodiment, the compound represented by formula (II) is 8-[6-(3-dimethylaminopropoxy)pyridin-3-yl]-3-methyl-1-(oxan-4-yl)imidazo[5,4-c]quinolin-2-one.

In another embodiment, the compound according to formula (II) is a pharmaceutically acceptable salt of 8-[6-(3-dimethylaminopropoxy)pyridin-3-yl]-3-methyl-1-(oxan-4-yl)imidazo[5,4-c]quinolin-2-one.

In another embodiment, the compound represented by formula (II) is 1-(cis-3-methoxycyclobutyl)-3-methyl-8-{6-[3-(pyrrolidin-1-yl)propoxy]pyridin-3-yl}-1,3-dihydro-2H-imidazo[4,5-c]quinolin-2-one, or a pharmaceutically acceptable salt thereof.

In another embodiment, the compound represented by formula (II) is 1-(cis-3-methoxycyclobutyl)-3-methyl-8-{6-[3-(pyrrolidin-1-yl)propoxy]pyridin-3-yl}-1,3-dihydro-2H-imidazo[4,5-c]quinolin-2-one.

In another embodiment, the compound according to formula (II) is a pharmaceutically acceptable salt of 1-(cis-3-methoxycyclobutyl)-3-methyl-8-{6-[3-(pyrrolidin-1-yl)propoxy]pyridin-3-yl}-1,3-dihydro-2H-imidazo[4,5-c]quinolin-2-one.

In a preferred embodiment, the ATM inhibitor used in the disclosure is a compound of formula (I), wherein the the compound is AZD1390 represented by the following formula:

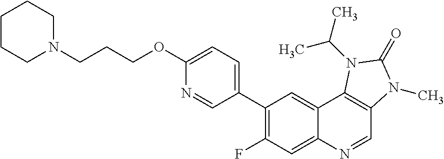 [SMILES: CC(C)n1c(=O)n(C)c2cnc3cc(F)c(-c4ccc(OCCCN5CCCCC5)nc4)cc3c21]

or a pharmaceutically acceptable salt thereof.

In another preferred embodiment, the ATM inhibitor used in the disclosure is a compound of formula (II), wherein the compound is AZD0156 represented by the following formula:

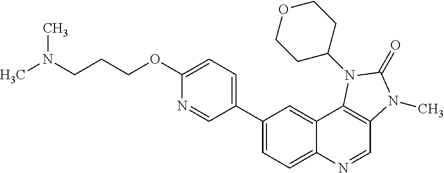

or a pharmaceutically acceptable salts thereof.

ATM inhibitors such as compounds of formula (I) and formula (II) may be prepared by methods known in the art such as disclosed in WO2017/046216 and WO2015/170081.

It will be understood that compounds of formula (I) or (II), and other ATM inhibitors mentioned herein, may encompass compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

In particular, as described in WO2018/167203, 7-fluoro-1-isopropyl-3-methyl-8-[6-[3-(1-piperidyl)propoxy]-3-pyridyl]imidazo[4,5-c]quinolin-2-one (AZD1390) may exist as 4,6-dideutero-7-fluoro-1-isopropyl-3-methyl-8-[6-[3-(1-piperidyl)propoxy]-3-pyridyl]imidazo[4,5-c]quinolin-2-one, with the chemical structure:

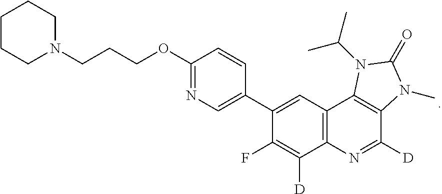 [SMILES: [2H]c1c(F)c(-c2ccc(OCCCN3CCCCC3)nc2)cc2c1nc([2H])c1c2n(C(C)C)c(=O)n1C]

Alternatively, as also described in WO2018/167203, AZD1390 may exist as 4-deutero-7-fluoro-1-isopropyl-3-methyl-8-[6-[3-(1-piperidyl)propoxy]-3-pyridyl]imidazo[4,5-c]quinolin-2-one, with the chemical structure:

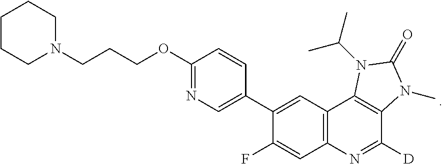 [SMILES: [2H]c1nc2cc(F)c(-c3ccc(OCCCN4CCCCC4)nc3)cc2c2c1n(C)c(=O)n2C(C)C]

Other examples of ATM inhibitors which may be used according to the present disclosure are:
8-[4-[3-(Dimethylamino)propoxy]phenyl]-1-isopropyl-3-methyl-imidazo[4,5-c]quinolin-2-one;
1-Isopropyl-3-methyl-8-[4-(3-pyrrolidin-1-ylpropoxy)phenyl]imidazo[4,5-c]quinolin-2-one;
8-[4-[3-(Azetidin-1-yl)propoxy]phenyl]-1-isopropyl-3-methyl-imidazo[4,5-c]quinolin-2-one;
8-[4-[3-(Azetidin-1-yl)propoxy]phenyl]-7-fluoro-1-isopropyl-3-methyl-imidazo[4,5-c]quinolin-2-one;
8-[4-[2-(Dimethylamino)ethoxy]phenyl]-1-isopropyl-3-methyl-imidazo[4,5-c]quinolin-2-one;
8-[4-[3-(Dimethylamino)propoxy]phenyl]-1-[(1S,3S)-3-methoxycyclopentyl]-3-methyl-imidazo[4,5-c]quinolin-2-one;
8-[4-[3-(Dimethylamino)propoxy]phenyl]-1-[(1R,3R)-3-methoxycyclopentyl]-3-methyl-imidazo[4,5-c]quinolin-2-one;
8-[4-[3-(Dimethylamino)propoxy]phenyl]-3-methyl-1-[(3R)-tetrahydropyran-3-yl]imidazo[4,5-c]quinolin-2-one;
8-[4-[3-(Dimethylamino)propoxy]phenyl]-3-methyl-1-[(3S)-tetrahydropyran-3-yl]imidazo[4,5-c]quinolin-2-one;
8-[4-[3-(Dimethylamino)propoxy]phenyl]-7-fluoro-3-methyl-1-[(3S)-tetrahydropyran-3-yl]imidazo[4,5-c]quinolin-2-one;
8-[4-[3-(Dimethylamino)propoxy]phenyl]-1-(cis-3-methoxycyclobutyl)-3-methyl-imidazo[4,5-c]quinolin-2-one;
8-[4-[3-(Dimethylamino)propoxy]phenyl]-7-fluoro-1-(cis-3-methoxycyclobutyl)-3-methyl-imidazo[4,5-c]quinolin-2-one;
8-[4-[3-(Dimethylamino)propoxy]phenyl]-7-fluoro-1-[trans-3-methoxycyclopentyl]-3-methyl-imidazo[4,5-c]quinolin-2-one;
3-Methyl-8-[4-(3-pyrrolidin-1-ylpropoxy)phenyl]-1-[(3S)-tetrahydropyran-3-yl]imidazo[4,5-c]quinolin-2-one;
3-Methyl-8-[4-(3-pyrrolidin-1-ylpropoxy)phenyl]-1-[(3R)-tetrahydropyran-3-yl]imidazo[4,5-c]quinolin-2-one;
3-Methyl-8-[4-[3-(1-piperidyl)propoxy]phenyl]-1-[(3S)-tetrahydrofuran-3-yl]imidazo[4,5-c]quinolin-2-one;
3-Methyl-8-[4-[3-(1-piperidyl)propoxy]phenyl]-1-tetrahydropyran-4-yl-imidazo[4,5-c]quinolin-2-one;
1-[trans-3-Methoxycyclopentyl]-3-methyl-8-[4-(3-pyrrolidin-1-ylpropoxy)phenyl]imidazo[4,5-c]quinolin-2-one;
8-[4-[3-(Dimethylamino)propoxy]phenyl]-1-(trans-3-methoxycyclobutyl)-3-methyl-imidazo[4,5-c]quinolin-2-one;
1-(trans-4-Methoxycyclohexyl)-3-methyl-8-[4-(3-pyrrolidin-1-ylpropoxy)phenyl]imidazo[4,5-c]quinolin-2-one;
8-[4-[3-(Azetidin-1-yl)propoxy]phenyl]-7-fluoro-1-[trans-3-methoxycyclopentyl]-3-methyl-imidazo[4,5-c]quinolin-2-one;
8-[4-[3-(Dimethylamino)propoxy]phenyl]-1-(cis-4-methoxycyclohexyl)-3-methyl-imidazo[4,5-c]quinolin-2-one;
8-[4-[3-(Dimethylamino)propoxy]phenyl]-1-(cis-4-methoxycyclohexyl)-3-methyl-imidazo[4,5-c]quinolin-2-one;
1-(cis-4-Methoxycyclohexyl)-3-methyl-8-[4-(3-pyrrolidin-1-ylpropoxy)phenyl]imidazo[4,5-c]quinolin-2-one;
8-[4-[3-(Dimethylamino)propoxy]phenyl]-1-[trans-3-methoxycyclohexyl]-3-methyl-imidazo[4,5-c]quinolin-2-one;
8-[4-[3-(Dimethylamino)propoxy]phenyl]-1-[trans-3-methoxycyclohexyl]-3-methyl-imidazo[4,5-c]quinolin-2-one;
1-[trans-3-Methoxycyclohexyl]-3-methyl-8-[4-(3-pyrrolidin-1-ylpropoxy)phenyl]imidazo[4,5-c]quinolin-2-one;
8-[4-[3-(Dimethylamino)propoxy]phenyl]-1-[cis-3-methoxycyclohexyl]-3-methyl-imidazo[4,5-c]quinolin-2-one;
8-[4-[3-(Dimethylamino)propoxy]phenyl]-1-[cis-3-methoxycyclohexyl]-3-methyl-imidazo[4,5-c]quinolin-2-one;
8-[4-[3-(Dimethylamino)propoxy]phenyl]-1-[cis-3-methoxycyclopentyl]-3-methyl-imidazo[4,5-c]quinolin-2-one;
8-[4-[3-(Dimethylamino)propoxy]phenyl]-7-fluoro-1-[cis-3-methoxycyclopentyl]-3-methyl-imidazo[4,5-c]quinolin-2-one;
1-[cis-3-Methoxycyclohexyl]-3-methyl-8-[4-(3-pyrrolidin-1-ylpropoxy)phenyl]imidazo[4,5-c]quinolin-2-one;
1-[cis-3-Methoxycyclohexyl]-3-methyl-8-[4-(3-pyrrolidin-1-ylpropoxy)phenyl]imidazo[4,5-c]quinolin-2-one;
1-[(1S,3S)-3-Methoxycyclopentyl]-3-methyl-8-[4-[3-(1-piperidyl)propoxy]phenyl]imidazo[4,5-c]quinolin-2-one;
1-(cis-3-Methoxycyclobutyl)-3-methyl-8-[4-(3-pyrrolidin-1-ylpropoxy)phenyl]imidazo[4,5-c]quinolin-2-one;
1-(trans-3-Methoxycyclobutyl)-3-methyl-8-[4-(3-pyrrolidin-1-ylpropoxy)phenyl]imidazo[4,5-c]quinolin-2-one;
1-(cis-3-Methoxycyclobutyl)-3-methyl-8-[4-[3-(1-piperidyl)propoxy]phenyl]imidazo[4,5-c]quinolin-2-one;
8-[4-[3-(Dimethylamino)propoxy]phenyl]-3-methyl-1-tetrahydropyran-4-yl-imidazo[4,5-c]quinolin-2-one;
8-[4-[3-(Dimethylamino)propoxy]phenyl]-7-fluoro-3-methyl-1-tetrahydropyran-4-yl-imidazo[4,5-c]quinolin-2-one;
7-Fluoro-3-methyl-8-[4-(3-pyrrolidin-1-ylpropoxy)phenyl]-1-tetrahydropyran-4-yl-imidazo[4,5-c]quinolin-2-one;
8-[4-[2-(Dimethylamino)ethoxy]phenyl]-3-methyl-1-[(3S)-tetrahydropyran-3-yl]imidazo[4,5-c]quinolin-2-one;
8-[4-[2-(Dimethylamino)ethoxy]phenyl]-3-methyl-1-[(3R)-tetrahydropyran-3-yl]imidazo[4,5-c]quinolin-2-one;
1-(3-(cis)Methoxycyclobutyl)-3-methyl-8-[4-(2-pyrrolidin-1-ylethoxy)phenyl]imidazo[4,5-c]quinolin-2-one;
3-Methyl-8-[4-(2-pyrrolidin-1-ylethoxy)phenyl]-1-[(3R)-tetrahydropyran-3-yl]imidazo[4,5-c]quinolin-2-one;
3-Methyl-8-[4-(2-pyrrolidin-1-ylethoxy)phenyl]-1-[(3S)-tetrahydropyran-3-yl]imidazo[4,5-c]quinolin-2-one;
8-[4-[2-(Dimethylamino)ethoxy]phenyl]-1-[(1S,3S)-3-methoxycyclopentyl]-3-methyl-imidazo[4,5-c]quinolin-2-one;
1-Cyclobutyl-8-[4-[2-(dimethylamino)ethoxy]phenyl]-3-methyl-imidazo[4,5-c]quinolin-2-one;
8-[4-[2-(Dimethylamino)ethoxy]phenyl]-3-methyl-1-tetrahydropyran-4-yl-imidazo[4,5-c]quinolin-2-one;
8-[4-[2-(Dimethylamino)ethoxy]phenyl]-1-(3-(cis)methoxycyclobutyl)-3-methyl-imidazo[4,5-c]quinolin-2-one;
8-[4-[2-(Dimethylamino)ethoxy]phenyl]-7-fluoro-3-methyl-1-tetrahydropyran-4-yl-imidazo[4,5-c]quinolin-2-one;
8-[6-[(3R)-3-(Dimethylamino)pyrrolidin-1-yl]-3-pyridyl]-1-isopropyl-3-methyl-imidazo[4,5-c]quinolin-2-one;
8-[6-[(3S)-3-(dimethylamino)pyrrolidin-1-yl]-3-pyridyl]-1-isopropyl-3-methyl-imidazo[4,5-c]quinolin-2-one;
8-[6-[4-(dimethylamino)-1-piperidyl]-3-pyridyl]-7-fluoro-1-isopropyl-3-methyl-imidazo[4,5-c]quinolin-2-one;
8-[6-[3-(dimethylamino)azetidin-1-yl]-3-pyridyl]-1-isopropyl-3-methyl-imidazo[4,5-c]quinolin-2-one;
8-[6-[(3R)-3-(Dimethylamino)pyrrolidin-1-yl]-3-pyridyl]-1-[(1S,3S)-3-methoxycyclopentyl]-3-methyl-imidazo[4,5-c]quinolin-2-one;
8-[6-[(3R)-3-(Dimethylamino)pyrrolidin-1-yl]-3-pyridyl]-7-fluoro-1-(cis-3-methoxycyclobutyl)-3-methyl-imidazo[4,5-c]quinolin-2-one;
8-[6-[(3R)-3-(dimethylamino)pyrrolidin-1-yl]-3-pyridyl]-3-methyl-1-[(3R)-tetrahydropyran-3-yl]imidazo[4,5-c]quinolin-2-one;
8-[6-[(3R)-3-(dimethylamino)pyrrolidin-1-yl]-3-pyridyl]-3-methyl-1-[(3S)-tetrahydrofuran-3-yl]imidazo[4,5-c]quinolin-2-one;
8-[6-[(3R)-3-(dimethylamino)pyrrolidin-1-yl]-3-pyridyl]-3-methyl-1-[(3S)-tetrahydropyran-3-yl]imidazo[4,5-c]quinolin-2-one;
8-[6-[(3R)-3-(dimethylamino)pyrrolidin-1-yl]-3-pyridyl]-1-(cis-3-methoxycyclobutyl)-3-methyl-imidazo[4,5-c]quinolin-2-one;
8-[6-[(3R)-3-(dimethylamino)pyrrolidin-1-yl]-3-pyridyl]-1-(trans-3-methoxycyclobutyl)-3-methyl-imidazo[4,5-c]quinolin-2-one;
8-[6-[(3R)-3-(dimethylamino)pyrrolidin-1-yl]-3-pyridyl]-1-(trans-4-methoxycyclohexyl)-3-methyl-imidazo[4,5-c]quinolin-2-one;
8-[6-[(3R)-3-(dimethylamino)pyrrolidin-1-yl]-3-pyridyl]-3-methyl-1-tetrahydropyran-4-yl-imidazo[4,5-c]quinolin-2-one;
8-[6-[(3R)-3-(dimethylamino)pyrrolidin-1-yl]-3-pyridyl]-7-fluoro-3-methyl-1-[(3R)-tetrahydropyran-3-yl]imidazo[4,5-c]quinolin-2-one;
8-[6-[(3R)-3-(dimethylamino)pyrrolidin-1-yl]-3-pyridyl]-7-fluoro-3-methyl-1-[(3S)-tetrahydropyran-3-yl]imidazo[4,5-c]quinolin-2-one;
8-[6-[(3S)-3-(dimethylamino)pyrrolidin-1-yl]-3-pyridyl]-3-methyl-1-[(3R)-tetrahydropyran-3-yl]imidazo[4,5-c]quinolin-2-one;
8-[6-[(3S)-3-(dimethylamino)pyrrolidin-1-yl]-3-pyridyl]-3-methyl-1-[(3S)-tetrahydropyran-3-yl]imidazo[4,5-c]quinolin-2-one;
8-[6-[(3S)-3-(dimethylamino)pyrrolidin-1-yl]-3-pyridyl]-1-(cis-3-methoxycyclobutyl)-3-methyl-imidazo[4,5-c]quinolin-2-one;
8-[6-[(3S)-3-(dimethylamino)pyrrolidin-1-yl]-3-pyridyl]-7-fluoro-3-methyl-1-[(3R)-tetrahydropyran-3-yl]imidazo[4,5-c]quinolin-2-one;
8-[6-[(3S)-3-(dimethylamino)pyrrolidin-1-yl]-3-pyridyl]-7-fluoro-3-methyl-1-[(3S)-tetrahydropyran-3-yl]imidazo[4,5-c]quinolin-2-one;
8-[6-[(3S)-3-(dimethylamino)pyrrolidin-1-yl]-3-pyridyl]-7-fluoro-1-(cis-3-methoxycyclobutyl)-3-methyl-imidazo[4,5-c]quinolin-2-one;
8-[6-[3-(dimethylamino)azetidin-1-yl]-3-pyridyl]-1-[trans-3-methoxycyclopentyl]-3-methyl-imidazo[4,5-c]quinolin-2-one;
8-[6-[3-(dimethylamino)azetidin-1-yl]-3-pyridyl]-3-methyl-1-[(3S)-tetrahydropyran-3-yl]imidazo[4,5-c]quinolin-2-one;
8-[6-[3-(dimethylamino)azetidin-1-yl]-3-pyridyl]-1-(trans-4-methoxycyclohexyl)-3-methyl-imidazo[4,5-c]quinolin-2-one;
1-cyclobutyl-8-[6-[3-(dimethylamino)azetidin-1-yl]-3-pyridyl]-3-methyl-imidazo[4,5-c]quinolin-2-one;
8-[6-[3-(dimethylamino)azetidin-1-yl]-3-pyridyl]-3-methyl-1-[(3R)-tetrahydropyran-3-yl]imidazo[4,5-c]quinolin-2-one;
8-[6-[3-(dimethylamino)azetidin-1-yl]-3-pyridyl]-3-methyl-1-tetrahydropyran-4-yl-imidazo[4,5-c]quinolin-2-one;
8-[6-[3-(dimethylamino)azetidin-1-yl]-3-pyridyl]-7-fluoro-1-[trans-3-methoxycyclopentyl]-3-methyl-imidazo[4,5-c]quinolin-2-one;
8-[6-[3-(dimethylamino)azetidin-1-yl]-3-pyridyl]-7-fluoro-1-(cis-3-methoxycyclobutyl)-3-methyl-imidazo[4,5-c]quinolin-2-one;
8-[6-[4-(dimethylamino)-1-piperidyl]-3-pyridyl]-1-[trans-3-methoxycyclopentyl]-3-methyl-imidazo[4,5-c]quinolin-2-one;
8-[6-[4-(dimethylamino)-1-piperidyl]-3-pyridyl]-3-methyl-1-[(3R)-tetrahydropyran-3-yl]imidazo[4,5-c]quinolin-2-one;
8-[6-[4-(dimethylamino)-1-piperidyl]-3-pyridyl]-3-methyl-1-[(3S)-tetrahydropyran-3-yl]imidazo[4,5-c]quinolin-2-one;
8-[6-[4-(dimethylamino)-1-piperidyl]-3-pyridyl]-1-(cis-3-methoxycyclobutyl)-3-methyl-imidazo[4,5-c]quinolin-2-one;
1-cyclobutyl-8-[6-[4-(dimethylamino)-1-piperidyl]-3-pyridyl]-3-methyl-imidazo[4,5-c]quinolin-2-one;
8-[6-[4-(dimethylamino)-1-piperidyl]-3-pyridyl]-3-methyl-1-(oxetan-3-yl)imidazo[4,5-c]quinolin-2-one;
8-[6-[4-(dimethylamino)-1-piperidyl]-3-pyridyl]-3-methyl-1-tetrahydropyran-4-yl-imidazo[4,5-c]quinolin-2-one;
8-[6-[4-(dimethylamino)-1-piperidyl]-3-pyridyl]-7-fluoro-3-methyl-1-[(3R)-tetrahydropyran-3-yl]imidazo[4,5-c]quinolin-2-one;
8-[6-[4-(dimethylamino)-1-piperidyl]-3-pyridyl]-7-fluoro-3-methyl-1-[(3S)-tetrahydropyran-3-yl]imidazo[4,5-c]quinolin-2-one;
8-[6-[4-(dimethylamino)-1-piperidyl]-3-pyridyl]-7-fluoro-1-(cis-3-methoxycyclobutyl)-3-methyl-imidazo[4,5-c]quinolin-2-one;
8-[6-[(3R)-3-(dimethylamino)-1-piperidyl]-3-pyridyl]-7-fluoro-1-(cis-3-methoxycyclobutyl)-3-methyl-imidazo[4,5-c]quinolin-2-one;
8-[6-[(3R)-3-(dimethylamino)pyrrolidin-1-yl]-3-pyridyl]-1-(cis-4-methoxycyclohexyl)-3-methyl-imidazo[4,5-c]quinolin-2-one;
8-[6-[4-(dimethylamino)-1-piperidyl]-3-pyridyl]-1-[(cis-3-methoxycyclohexyl]-3-methyl-imidazo[4,5-c]quinolin-2-one;
8-[6-[(3R)-3-(dimethylamino)pyrrolidin-1-yl]-3-pyridyl]-1-[cis-3-methoxycyclohexyl]-3-methyl-imidazo[4,5-c]quinolin-2-one;
8-[6-[3-(dimethylamino)azetidin-1-yl]-3-pyridyl]-1-[cis-3-methoxycyclohexyl]-3-methyl-imidazo[4,5-c]quinolin-2-one;
8-[6-[(3R)-3-(dimethylamino)pyrrolidin-1-yl]-3-pyridyl]-1-[trans-3-methoxycyclohexyl]-3-methyl-imidazo[4,5-c]quinolin-2-one;
8-[6-[4-(dimethylamino)-1-piperidyl]-3-pyridyl]-1-[trans-3-methoxycyclohexyl]-3-methyl-imidazo[4,5-c]quinolin-2-one;
8-[6-[3-(dimethylamino)azetidin-1-yl]-3-pyridyl]-1-[trans-3-methoxycyclohexyl]-3-methyl-imidazo[4,5-c]quinolin-2-one;
7-Fluoro-1-(cis-3-methoxycyclobutyl)-3-methyl-8-[6-[4-(methylamino)-1-piperidyl]-3-pyridyl]imidazo[4,5-c]quinolin-2-one;
3-methyl-8-[6-[4-(methylamino)-1-piperidyl]-3-pyridyl]-1-[(3R)-tetrahydropyran-3-yl]imidazo[4,5-c]quinolin-2-one;
3-methyl-8-[6-[4-(methylamino)-1-piperidyl]-3-pyridyl]-1-[(3S)-tetrahydropyran-3-yl]imidazo[4,5-c]quinolin-2-one;
1-(cis-3-methoxycyclobutyl)-3-methyl-8-[6-[4-(methylamino)-1-piperidyl]-3-pyridyl]imidazo[4,5-c]quinolin-2-one; and
3-methyl-8-[6-[4-(methylamino)-1-piperidyl]-3-pyridyl]-1-tetrahydropyran-4-yl-imidazo[4,5-c]quinolin-2-one.
(R)-8-(6-(Methoxymethyl)pyridin-3-yl)-3-methyl-1-(tetrahydro-2H-pyran-3-yl)-1H-imidazo[4,5-c]quinolin-2(3H)-one;
(S)-8-(6-(Methoxymethyl)pyridin-3-yl)-3-methyl-1-(tetrahydro-2H-pyran-3-yl)-1H-imidazo[4,5-c]quinolin-2(3H)-one;
(S)-7-Fluoro-8-(6-(methoxymethyl)pyridin-3-yl)-3-methyl-1-(tetrahydro-2H-pyran-3-yl)-1,3-dihydro-2H-imidazo[4,5-c]quinolin-2-one;
(R)-7-Fluoro-8-(2-fluoro-6-(methoxymethyl)pyridin-3-yl)-3-methyl-1-(tetrahydro-2H-pyran-3-yl)-1H-imidazo[4,5-c]quinolin-2(3H)-one;
(S)-7-Fluoro-8-(2-fluoro-6-(methoxymethyl)pyridin-3-yl)-3-methyl-1-(tetrahydro-2H-pyran-3-yl)-1H-imidazo[4,5-c]quinolin-2(3H)-one;
(R)-8-(2-Fluoro-6-(methoxymethyl)pyridin-3-yl)-3-methyl-1-(tetrahydro-2H-pyran-3-yl)-1H-imidazo[4,5-c]quinolin-2(3H)-one;
(S)-8-(2-Fluoro-6-(methoxymethyl)pyridin-3-yl)-3-methyl-1-(tetrahydro-2H-pyran-3-yl)-1H-imidazo[4,5-c]quinolin-2(3H)-one;
(R)-7-Fluoro-8-(6-(methoxymethyl)pyridin-3-yl)-3-methyl-1-(tetrahydro-2H-pyran-3-yl)-1H-imidazo[4,5-c]quinolin-2(3H)-one;
rac-1-(3,3-Dimethyltetrahydro-2H-pyran-4-yl)-8-(6-(methoxymethyl)pyridin-3-yl)-3-methyl-1,3-dihydro-2H-imidazo[4,5-c]quinolin-2-one;
(R)-1-(3,3-Dimethyltetrahydro-2H-pyran-4-yl)-8-(6-(methoxymethyl)pyridin-3-yl)-3-methyl-1,3-dihydro-2H-imidazo[4,5-c]quinolin-2-one;
(S)-1-(3,3-Dimethyltetrahydro-2H-pyran-4-yl)-8-(6-(methoxymethyl)pyridin-3-yl)-3-methyl-1,3-dihydro-2H-imidazo[4,5-c]quinolin-2-one;
(S)-8-(6-(Methoxymethyl)pyridin-3-yl)-1-(tetrahydro-2H-pyran-3-yl)-1,3-dihydro-2H-imidazo[4,5-c]quinolin-2-one;
7-fluoro-6-[6-(methoxymethyl)pyridin-3-yl]-4-{[(1S)-1-(1-methyl-1H-pyrazol-3-yl)ethyl]amino}quinoline-3-carboxamide;
7-fluoro-6-[6-(methoxymethyl)pyridin-3-yl]-4-{[(1R)-1-(1-methyl-1H-pyrazol-3-yl)ethyl]amino}quinoline-3-carboxamide;
7-fluoro-6-[2-fluoro-6-(methoxymethyl)pyridin-3-yl]-4-{[(1S)-1-(1-methyl-1H-pyrazol-3-yl)ethyl]amino}quinoline-3-carboxamide;
4-{[(1S)-1-(1,4-dimethyl-1H-pyrazol-3-yl)ethyl]amino}-7-fluoro-6-[2-fluoro-6-(methoxymethyl)pyridin-3-yl]quinoline-3-carboxamide;
4-{[(1S)-1-(1,4-dimethyl-1H-pyrazol-3-yl)ethyl]amino}-7-fluoro-6-[2-fluoro-6-(hydroxymethyl)pyridin-3-yl]quinoline-3-carboxamide;
4-{[(1S)-1-(1,4-dimethyl-1H-pyrazol-3-yl)ethyl]amino}-7-fluoro-6-[6-(methoxymethyl)pyridin-3-yl]quinoline-3-carboxamide;
4-{[(1R)-1-(1,4-dimethyl-1H-pyrazol-3-yl)ethyl]amino}-7-fluoro-6-[6-(methoxymethyl)pyridin-3-yl]quinoline-3-carboxamide;
7-fluoro-6-[6-(methoxymethyl)pyridin-3-yl]-4-{[(1S)-1-(1H-pyrazol-3-yl)ethyl]amino}quinoline-3-carboxamide;
7-fluoro-6-[6-(methoxymethyl)pyridin-3-yl]-4-{[(1R)-1-(1H-pyrazol-3-yl)ethyl]amino}quinoline-3-carboxamide;
7-fluoro-4-{[(1S)-1-(4-fluoro-1-methyl-1H-pyrazol-3-yl)ethyl]amino}-6-[6-(methoxymethyl)pyridin-3-yl]quinoline-3-carboxamide;
7-fluoro-4-{[(1R)-1-(4-fluoro-1-methyl-1H-pyrazol-3-yl)ethyl]amino}-6-[6-(methoxymethyl)pyridin-3-yl]quinoline-3-carboxamide;
7-fluoro-6-[6-(methoxymethyl)pyridin-3-yl]-4-{[(1S)-1-(4-methyl-1H-pyrazol-3-yl)ethyl]amino}quinoline-3-carboxamide;
7-fluoro-6-[6-(methoxymethyl)pyridin-3-yl]-4-{[(1R)-1-(4-methyl-1H-pyrazol-3-yl)ethyl]amino}quinoline-3-carboxamide;
7-fluoro-6-[6-(hydroxymethyl)pyridin-3-yl]-4-{[(1S)-1-(1-methyl-1H-pyrazol-3-yl)ethyl]amino}quinoline-3-carboxamide;
4-{[(1S)-1-(1,4-dimethyl-1H-pyrazol-3-yl)ethyl]amino}-7-fluoro-6-[6-(hydroxymethyl)pyridin-3-yl]quinoline-3-carboxamide;
4-{[(1R)-1-(1,4-dimethyl-1H-pyrazol-3-yl)ethyl]amino}-7-fluoro-6-[6-(hydroxymethyl)pyridin-3-yl]quinoline-3-carboxamide;
6-[2-fluoro-6-(methoxymethyl)pyridin-3-yl]-4-{[(1S)-1-(1-methyl-1H-pyrazol-3-yl)ethyl]amino}quinoline-3-carboxamide;
4-{[(1S)-1-(1,4-dimethyl-1H-pyrazol-3-yl)ethyl]amino}-6-[2-fluoro-6-(methoxymethyl)pyridin-3-yl]quinoline-3-carboxamide;
4-{[(1S)-1-(1,4-dimethyl-1H-pyrazol-3-yl)ethyl]amino}-6-[6-(methoxymethyl)pyridin-3-yl]quinoline-3-carboxamide;
4-{[(1R)-1-(1,4-dimethyl-1H-pyrazol-3-yl)ethyl]amino}-6-[6-(methoxymethyl)pyridin-3-yl]quinoline-3-carboxamide;
4-{[(1S)-1-(1,4-dimethyl-1H-pyrazol-3-yl)ethyl]amino}-6-[2-fluoro-6-(hydroxymethyl)pyridin-3-yl]quinoline-3-carboxamide;
6-[6-(hydroxymethyl)pyridin-3-yl]-4-{[(1S)-1-(1-methyl-1H-pyrazol-3-yl)ethyl]amino}quinoline-3-carboxamide;
4-{[(1S)-1-(1,4-dimethyl-1H-pyrazol-3-yl)ethyl]amino}-6-[6-(hydroxymethyl)pyridin-3-yl]quinoline-3-carboxamide;
4-{[(1R)-1-(1,4-dimethyl-1H-pyrazol-3-yl)ethyl]amino}-6-[6-(hydroxymethyl)pyridin-3-yl]quinoline-3-carboxamide;
4-{[(1S)-1-(4-fluoro-1-methyl-1H-pyrazol-3-yl)ethyl]amino}-6-[6-(methoxymethyl)pyridin-3-yl]quinoline-3-carboxamide;
6-[6-(methoxymethyl)pyridin-3-yl]-4-{[(1S)-1-(1-methyl-1H-pyrazol-3-yl)ethyl]amino}quinoline-3-carboxamide;
6-[6-(methoxymethyl)pyridin-3-yl]-4-{[(1R)-1-(1-methyl-1H-pyrazol-3-yl)ethyl]amino}quinoline-3-carboxamide;
6-[2-fluoro-6-(methoxymethyl)-3-pyridyl]-4-[[(1S)-1-(4-fluoro-1-methyl-pyrazol-3-yl)ethyl]amino]quinoline-3-carboxamide;
6-[6-(3-Dimethylaminopropoxy)pyridin-3-yl]-N-methyl-4-[[(1S)-1-(oxan-4-yl)ethyl]amino]cinnoline-3-carboxamide;
6-[6-(3-Dimethylaminopropoxy)pyridin-3-yl]-4-[[(1S)-1-(oxan-4-yl)ethyl]amino]cinnoline-3-carboxamide;
4-[[(1S)-1-(Oxan-4-yl)ethyl]amino]-6-[6-(3-pyrrolidin-1-ylpropoxy)pyridin-3-yl]cinnoline-3-carboxamide;
6-[6-(3-Methylaminopropoxy)pyridin-3-yl]-4-[[(1S)-1-(oxan-4-yl)ethyl]amino]cinnoline-3-carboxamide;
N-Methyl-6-[6-(3-methylaminopropoxy)pyridin-3-yl]-4-[[(1S)-1-(oxan-4-yl)ethyl]amino]cinnoline-3-carboxamide;
6-[6-(3-Dimethylaminopropoxy)pyridin-3-yl]-N-methyl-4-[[(1R)-1-(oxan-4-yl)ethyl]amino]cinnoline-3-carboxamide;
N,N-Dimethyl-3-[[5-(3-methyl-2-oxo-1-tetrahydropyran-4-yl-imidazo[4,5-c]quinolin-8-yl)-2-pyridyl]oxy]propan-1-amine oxide;
8-(6-(3-(4-Fluoropiperidin-1-yl)propoxy)pyridin-3-yl)-1-isopropyl-3-methyl-1,3-dihydro-2H-imidazo[4,5-c]quinolin-2-one;
(S)-8-(6-(3-(3-Fluoropyrrolidin-1-yl)propoxy)pyridin-3-yl)-1-isopropyl-3-methyl-1,3-dihydro-2H-imidazo[4,5-c]quinolin-2-one;
(R)-8-(6-(3-(3-Fluoropyrrolidin-1-yl)propoxy)pyridin-3-yl)-1-isopropyl-3-methyl-1,3-dihydro-2H-imidazo[4,5-c]quinolin-2-one;
8-[6-[3-(Dimethylamino)propoxy]-3-pyridyl]-3-methyl-1-tetrahydropyran-4-yl-imidazo[4,5-c]cinnolin-2-one;
8-[6-[3-(dimethylamino)propoxy]-3-pyridyl]-1-(cis-3-methoxycyclobutyl)-3-methyl-imidazo[4,5-c]cinnolin-2-one;
8-[6-[3-(Dimethylamino)propoxy]-3-pyridyl]-3-methyl-1-[(3S)-tetrahydropyran-3-yl]imidazo[4,5-c]cinnolin-2-one;
8-[6-[3-(Dimethylamino)propoxy]-3-pyridyl]-3-methyl-1-[(3R)-tetrahydropyran-3-yl]imidazo[4,5-c]cinnolin-2-one;
3-Methyl-8-[6-[3-(1-piperidyl)propoxy]-3-pyridyl]-1-tetrahydropyran-4-yl-imidazo[4,5-c]cinnolin-2-one;
8-[4-[3-(dimethylamino)propoxy]phenyl]-1-isopropyl-3-methyl-imidazo[4,5-c]cinnolin-2-one;
1-Isopropyl-3-methyl-8-[6-[3-(1-piperidyl)propoxy]-3-pyridyl]imidazo[4,5-c]cinnolin-2-one;
8-[6-[3-(dimethylamino)propoxy]-3-pyridyl]-1-isopropyl-3-methyl-imidazo[4,5-c]cinnolin-2-one;
1-Isopropyl-3-methyl-8-[4-(3-pyrrolidin-1-ylpropoxy)phenyl]imidazo[4,5-c]cinnolin-2-one;
1-isopropyl-3-methyl-8-(4-(3-(piperidin-1-yl)propoxy)phenyl)-1,3-dihydro-2H-imidazo[4,5-c]cinnolin-2-one;
8-[6-[3-(dimethylamino)propoxy]-3-pyridyl]-1-(trans-3-methoxycyclobutyl)-3-methyl-imidazo[4,5-c]cinnolin-2-one;
8-[6-[3-(4-Fluoro-1-piperidyl)propoxy]-3-pyridyl]-1-isopropyl-3-methyl-imidazo[4,5-c]cinnolin-2-one;
8-[6-[3-[(3R)-3-Fluoropyrrolidin-1-yl]propoxy]-3-pyridyl]-1-isopropyl-3-methyl-imidazo[4,5-c]cinnolin-2-one;
1-isopropyl-3-methyl-8-[2-methyl-6-[3-(1-piperidyl)propoxy]-3-pyridyl]imidazo[4,5-c]cinnolin-2-one;
1-(trans-3-Methoxycyclobutyl)-3-methyl-8-[4-[3-(1-piperidyl)propoxy]phenyl]imidazo[4,5-c]cinnolin-2-one;
8-[4-[3-(Dimethylamino)propoxy]phenyl]-1-(trans-3-methoxycyclobutyl)-3-methyl-imidazo[4,5-c]cinnolin-2-one;
8-[6-[3-(4-Fluoro-1-piperidyl)propoxy]-3-pyridyl]-1-(trans-3-methoxycyclobutyl)-3-methyl-imidazo[4,5-c]cinnolin-2-one;
8-[6-[3-(4-Fluoro-1-piperidyl)propoxy]-3-pyridyl]-1-(cis-3-methoxycyclobutyl)-3-methyl-imidazo[4,5-c]cinnolin-2-one;
1-(cis-3-methoxycyclobutyl)-3-methyl-8-[4-[3-(1-piperidyl)propoxy]phenyl]imidazo[4,5-c]cinnolin-2-one;
8-[6-[3-[(3R)-3-Fluoropyrrolidin-1-yl]propoxy]-3-pyridyl]-1-(cis-3-methoxycyclobutyl)-3-methyl-imidazo[4,5-c]cinnolin-2-one;
8-[4-[3-(Dimethylamino)propoxy]phenyl]-3-methyl-1-[(3R)-tetrahydropyran-3-yl]imidazo[4,5-c]cinnolin-2-one;
3-Methyl-8-[4-[3-(1-piperidyl)propoxy]phenyl]-1-[(3R)-tetrahydropyran-3-yl]imidazo[4,5-c]cinnolin-2-one;
8-[6-[3-(4-Fluoro-1-piperidyl)propoxy]-3-pyridyl]-3-methyl-1-[(3R)-tetrahydropyran-3-yl]imidazo[4,5-c]cinnolin-2-one;
8-[4-[3-(Dimethylamino)propoxy]phenyl]-3-methyl-1-(1-methylcyclopropyl)imidazo[4,5-c]cinnolin-2-one;
3-Methyl-1-(1-methylcyclopropyl)-8-[4-[3-(1-piperidyl)propoxy]phenyl]imidazo[4,5-c]cinnolin-2-one;
8-[6-[3-(4-Fluoro-1-piperidyl)propoxy]-3-pyridyl]-3-methyl-1-(1-methylcyclopropyl)imidazo[4,5-c]cinnolin-2-one;
3-Methyl-1-(1-methylcyclopropyl)-8-[6-[3-(1-piperidyl)propoxy]-3-pyridyl]imidazo[4,5-c]cinnolin-2-one;
8-[4-[3-(dimethylamino)propoxy]phenyl]-1-[(1R,3R)-3-methoxycyclopentyl]-3-methyl-imidazo[4,5-c]cinnolin-2-one;
1-[(1S,3S)-3-methoxycyclopentyl]-3-methyl-8-[4-[3-(1-piperidyl)propoxy]phenyl]imidazo[4,5-c]cinnolin-2-one;
8-[4-[3-(Dimethylamino)propoxy]phenyl]-1-[(1S,3S)-3-methoxycyclopentyl]-3-methyl-imidazo[4,5-c]cinnolin-2-one;
1-[(1R,3R)-3-Methoxycyclopentyl]-3-methyl-8-[6-[3-(1-piperidyl)propoxy]-3-pyridyl]imidazo[4,5-c]cinnolin-2-one;
8-[6-[3-[(3S)-3-Fluoropyrrolidin-1-yl]propoxy]-3-pyridyl]-1-[(1R,3R)-3-methoxycyclopentyl]-3-methyl-imidazo[4,5-c]cinnolin-2-one;
1-[(1S,3S)-3-Methoxycyclopentyl]-3-methyl-8-[6-[3-(1-piperidyl)propoxy]-3-pyridyl]imidazo[4,5-c]cinnolin-2-one;
8-[4-[3-(Dimethylamino)propoxy]phenyl]-1-(cis-3-methoxycyclobutyl)-3-methyl-imidazo[4,5-c]cinnolin-2-one;
1-(cis-3-Methoxycyclobutyl)-3-methyl-8-[6-[3-(1-piperidyl)propoxy]-3-pyridyl]imidazo[4,5-c]cinnolin-2-one;
8-[6-[3-[(3S)-3-Fluoropyrrolidin-1-yl]propoxy]-3-pyridyl]-1-(trans-3-methoxycyclobutyl)-3-methyl-imidazo[4,5-c]cinnolin-2-one;
8-[6-[3-(dimethylamino)propoxy]-3-pyridyl]-1-[(4S)-3,3-dimethyltetrahydropyran-4-yl]-3-methyl-imidazo[4,5-c]cinnolin-2-one;
8-[6-[3-(dimethylamino)propoxy]-3-pyridyl]-1-[(4R)-3,3-dimethyltetrahydropyran-4-yl]-3-methyl-imidazo[4,5-c]cinnolin-2-one
1-[3,3-dimethyltetrahydropyran-4-yl]-3-methyl-8-{4-[3-(1-piperidinyl)propoxy]phenyl}-1,3-dihydro-2H-imidazo[4,5-c]cinnolin-2-one;
8-[4-[3-(dimethylamino)propoxy]phenyl]-1-[(4S)-3,3-dimethyltetrahydropyran-4-yl]-3-methyl-imidazo[4,5-c]cinnolin-2-one;
8-[4-[3-(dimethylamino)propoxy]phenyl]-1-[(4R)-3,3-dimethyltetrahydropyran-4-yl]-3-methyl-imidazo[4,5-c]cinnolin-2-one;
8-[4-[3-(dimethylamino)propoxy]phenyl]-7-fluoro-1-isopropyl-3-methyl-imidazo[4,5-c]cinnolin-2-one;
8-[6-[3-(Dimethylamino)propoxy]-3-pyridyl]-7-fluoro-1-isopropyl-3-methyl-imidazo[4,5-c]cinnolin-2-one;
7-Fluoro-1-isopropyl-3-methyl-8-[6-[3-(1-piperidyl)propoxy]-3-pyridyl]imidazo[4,5-c]cinnolin-2-one;
7-fluoro-1-isopropyl-3-methyl-8-[4-[3-(1-piperidyl)propoxy]phenyl]imidazo[4,5-c]cinnolin-2-one;
8-[4-[3-(dimethylamino)propoxy]phenyl]-1-isopropyl-3,7-dimethyl-imidazo[4,5-c]cinnolin-2-one;
1-isopropyl-3,7-dimethyl-8-[6-[3-(1-piperidyl)propoxy]-3-pyridyl]imidazo[4,5-c]cinnolin-2-one;
8-[6-[3-(dimethylamino)propoxy]-3-pyridyl]-1-isopropyl-3,7-dimethyl-imidazo[4,5-c]cinnolin-2-one;
1-Isopropyl-3,7-dimethyl-8-[4-[3-(1-piperidyl)propoxy]phenyl]imidazo[4,5-c]cinnolin-2-one;
3-(difluoromethyl)-8-[6-[3-(dimethylamino)propoxy]-3-pyridyl]-1-isopropyl-imidazo[4,5-c]cinnolin-2-one;
8-[6-[(3R)-3-(Dimethylamino)pyrrolidin-1-yl]-3-pyridyl]-1-isopropyl-3-methyl-imidazo[4,5-c]cinnolin-2-one;
8-[6-[4-(Dimethylamino)-1-piperidyl]-3-pyridyl]-1-isopropyl-3-methyl-imidazo[4,5-c]cinnolin-2-one;
8-[6-[(3R)-3-(dimethylamino)pyrrolidin-1-yl]-3-pyridyl]-3-methyl-1-[(3R)-tetrahydropyran-3-yl]imidazo[4,5-c]cinnolin-2-one;
8-[6-[4-(dimethylamino)-1-piperidyl]-3-pyridyl]-1-[3,3-dimethyltetrahydropyran-4-yl]-3-methyl-imidazo[4,5-c]cinnolin-2-one;
8-[6-[(3R)-3-(dimethylamino)pyrrolidin-1-yl]-3-pyridyl]-1-[3,3-dimethyltetrahydropyran-4-yl]-3-methyl-imidazo[4,5-c]cinnolin-2-one;
8-[6-[(3R)-3-(Dimethylamino)pyrrolidin-1-yl]-3-pyridyl]-1-(trans-3-methoxycyclobutyl)-3-methyl-imidazo[4,5-c]cinnolin-2-one;
8-[6-[(3R)-3-(Dimethylamino)pyrrolidin-1-yl]-3-pyridyl]-1-(cis-3-methoxycyclobutyl)-3-methyl-imidazo[4,5-c]cinnolin-2-one;
8-[6-[3-(dimethylamino)pyrrolidin-1-yl]-3-pyridyl]-3-methyl-1-tetrahydropyran-4-yl-imidazo[4,5-c]cinnolin-2-one;
8-[6-[(3R)-3-(Dimethylamino)pyrrolidin-1-yl]-3-pyridyl]-1-[(1R,3R)-3-methoxycyclopentyl]-3-methyl-imidazo[4,5-c]cinnolin-2-one;
8-[6-[(3R)-3-(Dimethylamino)pyrrolidin-1-yl]-3-pyridyl]-1-[(1S,3S)-3-methoxycyclopentyl]-3-methyl-imidazo[4,5-c]cinnolin-2-one;
8-[6-[4-(Dimethylamino)-1-piperidyl]-3-pyridyl]-1-(cis-3-methoxycyclobutyl)-3-methyl-imidazo[4,5-c]cinnolin-2-one;
8-[4-[4-(Dimethylamino)-1-piperidyl]phenyl]-1-isopropyl-3-methyl-imidazo[4,5-c]cinnolin-2-one;
8-[4-[4-(dimethylamino)-1-piperidyl]phenyl]-1-[3,3-dimethyltetrahydropyran-4-yl]-3-methyl-imidazo[4,5-c]cinnolin-2-one;
8-[4-[4-(Dimethylamino)-1-piperidyl]phenyl]-1-[(1R,3R)-3-methoxycyclopentyl]-3-methyl-imidazo[4,5-c]cinnolin-2-one;
8-[4-[4-(Dimethylamino)-1-piperidyl]phenyl]-1-[(1S,3S)-3-methoxycyclopentyl]-3-methyl-imidazo[4,5-c]cinnolin-2-one;
8-[4-[4-(Dimethylamino)-1-piperidyl]phenyl]-3-methyl-1-[(3R)-tetrahydropyran-3-yl]imidazo[4,5-c]cinnolin-2-one;
8-[6-[(3R)-3-(Dimethylamino)pyrrolidin-1-yl]-3-pyridyl]-7-fluoro-1-isopropyl-3-methyl-imidazo[4,5-c]cinnolin-2-one;
8-[4-[4-(Dimethylamino)-1-piperidyl]phenyl]-7-fluoro-1-isopropyl-3-methyl-imidazo[4,5-c]cinnolin-2-one;
8-[6-[(3R)-3-(dimethylamino)pyrrolidin-1-yl]-3-pyridyl]-1-isopropyl-3,7-dimethyl-imidazo[4,5-c]cinnolin-2-one;
8-[4-[4-(dimethylamino)-1-piperidyl]phenyl]-1-isopropyl-3,7-dimethyl-imidazo[4,5-c]cinnolin-2-one; and
8-[6-[3-(dimethylamino)propoxy]-2-fluoro-3-pyridyl]-1-isopropyl-3-methyl-imidazo[4,5-c]cinnolin-2-one.

### 5. Combination of Antibody-Drug Conjugate and ATM Inhibitor

In a first combination embodiment of the disclosure, the anti-HER2 antibody-drug conjugate which is combined with the ATM inhibitor is an antibody-drug conjugate in which a drug-linker represented by the following formula:

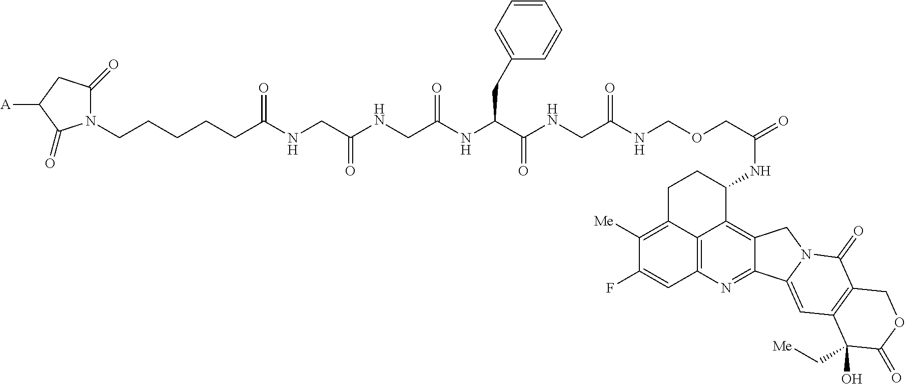

wherein A represents the connecting position to an antibody, is conjugated to an anti-HER2 antibody via a thioether bond.

In another combination embodiment, the anti-HER2 antibody-drug conjugate as defined above for the first combination embodiment is combined with an ATM inhibitor which is a compound represented by the following formula (I):

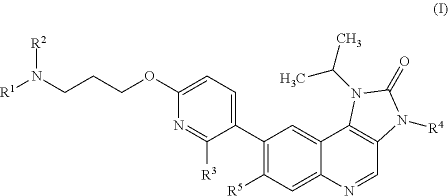

wherein:

R¹ is methyl;

R² is hydrogen or methyl; or R¹ and R² together with the nitrogen atom to which they are bonded form an azetidinyl, pyrrolidinyl or piperidinyl ring;

R³ is hydrogen or fluoro;

R⁴ is hydrogen or methyl; and

R⁵ is hydrogen or fluoro, or a pharmaceutically acceptable salt thereof;

In another combination embodiment, the anti-HER2 antibody-drug conjugate as defined above is combined with an ATM inhibitor which is a compound represented by formula (I) as defined above wherein, in formula (I), R¹ and R² are both methyl; or R¹ and R² together with the nitrogen atom to which they are bonded form an azetidinyl, pyrrolidinyl or piperidinyl ring;

In another combination embodiment, the anti-HER2 antibody-drug conjugate as defined above is combined with an ATM inhibitor as defined above wherein, in formula (I), R¹ and R² together with the nitrogen atom to which they are bonded form an azetidinyl, pyrrolidinyl or piperidinyl ring;

In another combination embodiment, the anti-HER2 antibody-drug conjugate as defined above is combined with an ATM inhibitor as defined above wherein, in formula (I), R³ is hydrogen;

In another combination embodiment, the anti-HER2 antibody-drug conjugate as defined above is combined with an ATM inhibitor as defined above wherein, in formula (I), R⁴ is methyl;

In another combination embodiment, the anti-HER2 antibody-drug conjugate as defined above is combined with an ATM inhibitor as defined above wherein, in formula (I), R⁵ is fluoro;

In another combination embodiment, the anti-HER2 antibody-drug conjugate as defined above is combined with an ATM inhibitor as defined above, wherein, in formula (I):

R¹ is methyl;

R² is methyl; or R¹ and R² together with the nitrogen atom to which they are bonded form an azetidinyl, pyrrolidinyl or piperidinyl ring;

R³ is hydrogen or fluoro;

R⁴ is methyl; and

R⁵ is hydrogen or fluoro;

In another combination embodiment, the anti-HER2 antibody-drug conjugate as defined above is combined with an ATM inhibitor as defined above, wherein the ATM inhibitor is AZD1390 represented by the following formula:

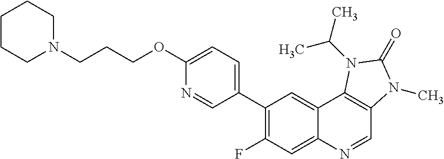 [SMILES: CC(C)n1c(=O)n(C)c2cnc3cc(F)c(-c4ccc(OCCCN5CCCCC5)nc4)cc3c21]

or a pharmaceutically acceptable salt thereof.

In an embodiment of each of the combination embodiments described above, the anti-HER2 antibody comprises a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence consisting of amino acid residues 1 to 3 of SEQ ID NO: 7 and CDRL3 consisting of an amino acid sequence represented by SEQ ID NO: 8. In another embodiment of each of the combination embodiments described above, the anti-HER2 antibody comprises a heavy chain comprising a heavy chain variable region consisting of an amino acid sequence represented by SEQ ID NO: 9 and a light chain comprising a light chain variable region consisting of an amino acid sequence represented by SEQ ID NO: 10. In another embodiment of each of the combination embodiments described above, the anti-HER2 antibody comprises a heavy chain consisting of an amino acid sequence represented by SEQ ID NO: 1 and a light chain consisting of an amino acid sequence represented by SEQ ID NO: 2. In another embodiment of each of the combination embodiments described above, the anti-HER2 antibody comprises a heavy chain consisting of an amino acid sequence represented by SEQ ID NO: 11 and a light chain consisting of an amino acid sequence represented by SEQ ID NO: 2.

In a particularly preferred combination embodiment of the disclosure, the anti-HER2 antibody-drug conjugate is trastuzumab deruxtecan (DS-8201) and the ATM inhibitor is the compound represented by the following formula:

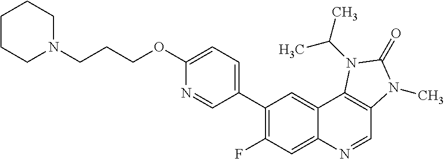 [SMILES: CC(C)n1c(=O)n(C)c2cnc3cc(F)c(-c4ccc(OCCCN5CCCCC5)nc4)cc3c21]

also identified as AZD1390.

### 6. Therapeutic Combined Use and Method

Described in the following are a pharmaceutical product and a therapeutic use and method wherein the anti-HER2 antibody-drug conjugate according to the present disclosure and an ATM inhibitor are administered in combination.

The pharmaceutical product and therapeutic use and method of the present disclosure may be characterized in that the anti-HER2 antibody-drug conjugate and the ATM inhibitor are separately contained as active components in different formulations, and are administered simultaneously or at different times, or characterized in that the antibody-drug conjugate and the ATM inhibitor are contained as active components in a single formulation and administered.

In the pharmaceutical product and therapeutic method of the present disclosure, a single ATM inhibitor used in the present disclosure can be administered in combination with the anti-HER2 antibody-drug conjugate, or two or more different ATM inhibitors can be administered in combination with the antibody-drug conjugate.

The pharmaceutical product and therapeutic method of the present disclosure can be used for treating cancer, and can be preferably used for treating at least one cancer selected from the group consisting of breast cancer (including triple negative breast cancer and luminal breast cancer), gastric cancer (also called gastric adenocarcinoma), colorectal cancer (also called colon and rectal cancer, and including colon cancer and rectal cancer), lung cancer (including small cell lung cancer and non-small cell lung cancer), esophageal cancer, head-and-neck cancer (including salivary gland cancer and pharyngeal cancer), esophagogastric junction adenocarcinoma, biliary tract cancer (including bile duct cancer), Paget's disease, pancreatic cancer, ovarian cancer, uterine carcinosarcoma, urothelial cancer, prostate cancer, bladder cancer, gastrointestinal stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioma, glioblastoma multiforme, osteosarcoma, sarcoma, and melanoma, and can be more preferably used for treating at least one cancer selected from the group consisting of breast cancer, gastric cancer, colorectal cancer, lung cancer (preferably non-small cell lung cancer), pancreatic cancer, ovarian cancer, prostate cancer, and kidney cancer.

The presence or absence of HER2 tumor markers can be determined, for example, by collecting tumor tissue from a cancer patient to prepare a formalin-fixed, paraffin-embedded (FFPE) specimen and subjecting the specimen to a test for gene products (proteins), for example, with an immunohistochemical (IHC) method, a flow cytometer, or Western blotting, or to a test for gene transcription, for example, with an in situ hybridization (ISH) method, a quantitative PCR method (q-PCR), or microarray analysis, or by collecting cell-free circulating tumor DNA (ctDNA) from a cancer patient and subjecting the ctDNA to a test with a method such as next-generation sequencing (NGS).

The pharmaceutical product and therapeutic method of the present disclosure can be used for cancer, which may be HER2-overexpressing cancer (high or moderate) or may be HER2 low-expressing cancer.

In the present disclosure, the term “HER2-overexpressing cancer” is not particularly limited as long as it is recognized as HER2-overexpressing cancer by those skilled in the art. Preferred examples of the HER2-overexpressing cancer can include cancer given a score of 3+ for the expression of HER2 in an IHC method, and cancer given a score of 2+ for the expression of HER2 in an IHC method and determined as positive for the expression of HER2 in an in situ hybridization method (ISH). The in situ hybridization method of the present disclosure includes a fluorescence in situ hybridization method (FISH) and a dual color in situ hybridization method (DISH).

In the present disclosure, the term “HER2 low-expressing cancer” is not particularly limited as long as it is recognized as HER2 low-expressing cancer by those skilled in the art. Preferred examples of the HER2 low-expressing cancer can include cancer given a score of 2+ for the expression of HER2 in an IHC method and determined as negative for the expression of HER2 in an in situ hybridization method, and cancer given a score of 1+ for the expression of HER2 in an IHC method.

The method for scoring the degree of HER2 expression by the IHC method, or the method for determining positivity or negativity to HER2 expression by the in situ hybridization method is not particularly limited as long as it is recognized by those skilled in the art. Examples of the method can include a method described in the 4th edition of the guidelines for HER2 testing, breast cancer (developed by the Japanese Pathology Board for Optimal Use of HER2 for Breast Cancer).

The cancer, particularly in regard to the treatment of breast cancer, may be HER2-overexpressing (high or moderate) or low-expressing breast cancer, or triple-negative breast cancer, and/or may have a HER2 status score of IHC 3+, IHC 2+, IHC 1+ or IHC >0 and <1+.

The pharmaceutical product and therapeutic method of the present disclosure can be preferably used for a mammal, but are more preferably used for a human.

The antitumor effect of the pharmaceutical product and therapeutic method of the present disclosure can be confirmed by transplanting cancer cells to a test subject animal to prepare a model and measuring reduction in tumor volume or life-prolonging effect by application of the pharmaceutical product and therapeutic method of the present disclosure. And then, the effect of combined use of the antibody-drug conjugate used in the present disclosure and an ATM inhibitor can be confirmed by comparing antitumor effect with single administration of the antibody-drug conjugate used in the present disclosure and that of the ATM inhibitor.

The antitumor effect of the pharmaceutical product and therapeutic method of the present disclosure can be confirmed in a clinical trial using any of an evaluation method with Response Evaluation Criteria in Solid Tumors (RECIST), a WHO evaluation method, a Macdonald evaluation method, body weight measurement, and other approaches, and can be determined on the basis of indexes of complete response (CR), partial response (PR); progressive disease (PD), objective response rate (ORR), duration of response (DoR), progression-free survival (PFS), overall survival (OS), and so on.

By using the above methods, the superiority in antitumor effect of the pharmaceutical product and therapeutic method of the present disclosure to existing pharmaceutical products and therapeutic methods for cancer treatment can be confirmed.

The pharmaceutical product and therapeutic method of the present disclosure can delay development of cancer cells, inhibit growth thereof, and further kill cancer cells. These effects can allow cancer patients to be free from symptoms caused by cancer or achieve improvement in quality of life (QOL) of cancer patients and attain a therapeutic effect by sustaining the lives of the cancer patients. Even if the pharmaceutical product and therapeutic method of the present disclosure do not accomplish killing cancer cells, they can achieve higher QOL of cancer patients while achieving longer-term survival, by inhibiting or controlling the growth of cancer cells.

The pharmaceutical product of the present disclosure can be expected to exert a therapeutic effect by application as systemic therapy to patients, and additionally, by local application to cancer tissues.

The pharmaceutical product of the present disclosure can be administered containing at least one pharmaceutically suitable ingredient. Pharmaceutically suitable ingredients can be suitably selected and applied from formulation additives or the like that are generally used in the art, in accordance with the dosage, administration concentration, or the like of the antibody-drug conjugate used in the present disclosure and an ATM inhibitor. The anti-HER2 antibody-drug conjugate used in the present disclosure can be administered, for example, as a pharmaceutical product containing a buffer such as histidine buffer, a vehicle such as sucrose and trehalose, and a surfactant such as Polysorbates 80 and 20. The pharmaceutical product containing the antibody-drug conjugate used in the present disclosure can be preferably used as an injection, can be more preferably used as an aqueous injection or a lyophilized injection, and can be even more preferably used as a lyophilized injection.

In the case that the pharmaceutical product containing the anti-HER2 antibody-drug conjugate used in the present disclosure is an aqueous injection, the aqueous injection can be preferably diluted with a suitable diluent and then given as an intravenous infusion. Examples of the diluent can include dextrose solution and physiological saline, dextrose solution can be preferably exemplified, and 5% dextrose solution can be more preferably exemplified.

In the case that the pharmaceutical product of the present disclosure is a lyophilized injection, a required amount of the lyophilized injection dissolved in advance in water for injection can be preferably diluted with a suitable diluent and then given as an intravenous infusion. Examples of the diluent can include dextrose solution and physiological saline, dextrose solution can be preferably exemplified, and 5% dextrose solution can be more preferably exemplified.

Examples of the administration route applicable to administration of the pharmaceutical product of the present disclosure can include intravenous, intradermal, subcutaneous, intramuscular, and intraperitoneal routes, and intravenous routes are preferred.

The anti-HER2 antibody-drug conjugate used in the present disclosure can be administered to a human with intervals of 1 to 180 days, can be preferably administered with intervals of a week, two weeks, three weeks, or four weeks, and can be more preferably administered with intervals of three weeks. The anti-HER2 antibody-drug conjugate used in the present disclosure can be administered in a dose of about 0.001 to 100 mg/kg per administration, and can be preferably administered in a dose of 0.8 to 12.4 mg/kg per administration. For example, the anti-HER2 antibody-drug conjugate can be administered once every three weeks at a dose of 0.8 mg/kg, 1.6 mg/kg, 3.2 mg/kg, 5.4 mg/kg, 6.4 mg/kg, 7.4 mg/kg, or 8 mg/kg, and can be preferably administered once every three weeks at a dose of 5.4 mg/kg or 6.4 mg/kg.

The compound of formula (I) or (II) will normally be administered to a warm-blooded animal at a unit dose within the range 2.5-5000 mg/m² body area of the animal, or approximately 0.05-100 mg/kg, and this normally provides a therapeutically-effective dose. A unit dose form such as a tablet or capsule will usually contain, for example 0.1-250 mg of active ingredient. The daily dose will necessarily be varied depending upon the host treated, the particular route of administration, any therapies being co-administered, and the severity of the illness being treated. Accordingly the practitioner who is treating any particular patient may determine the optimum dosage.

The pharmaceutical products may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing), or as a suppository for rectal dosing. The compositions may be obtained by conventional procedures well known in the art. Compositions intended for oral use may contain additional components, for example, one or more colouring, sweetening, flavouring and/or preservative agents.

The size of the dose required for the therapeutic treatment of a particular disease state will necessarily be varied depending on the subject treated, the route of administration and the severity of the illness being treated. A compound of formula (I) or (II) will normally be administered to a warm-blooded animal at a unit dose within the range 2.5-5000 mg/m² body area of the animal, or approximately 0.05-100 mg/kg, and this normally provides a therapeutically-effective dose. A unit dose form such as a tablet or capsule will usually contain, for example 0.1-250 mg of active ingredient.

The pharmaceutical product and therapeutic method of the present disclosure can be used as adjuvant chemotherapy combined with surgery operation. The pharmaceutical product of the present disclosure may be administered for the purpose of reducing tumor size before surgical operation (referred to as preoperative adjuvant chemotherapy or neoadjuvant therapy), or may be administered for the purpose of preventing recurrence of tumor after surgical operation (referred to as postoperative adjuvant chemotherapy or adjuvant therapy).

### EXAMPLES

The present disclosure is specifically described in view of the examples shown below. However, the present disclosure is not limited to these. Further, it is by no means to be interpreted in a limited way.

### Example 1: Production of Antibody-Drug Conjugate

In accordance with a production method described in WO2015/115091 and using an anti-HER2 antibody (an antibody comprising a heavy chain consisting of an amino acid sequence represented by SEQ ID NO: 11 (amino acid residues 1 to 449 of SEQ ID NO: 1) and a light chain consisting of an amino acid sequence consisting of all amino acid residues 1 to 214 of SEQ ID NO: 2), an anti-HER2 antibody-drug conjugate in which a drug-linker represented by the following formula:

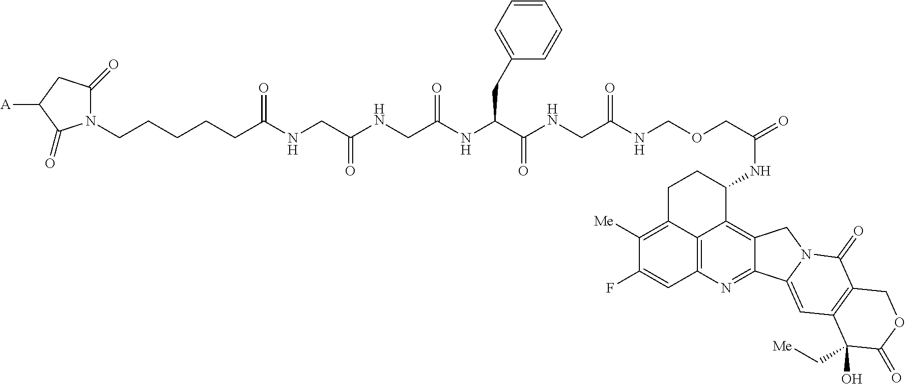

wherein A represents the connecting position to an antibody,

is conjugated to the anti-HER2 antibody via a thioether bond was produced (DS-8201: trastuzumab deruxtecan). The DAR of the antibody-drug conjugate is 7.7 or 7.8.

### Example 2: Production of ATM Inhibitor

In accordance with a production method described in WO2017/046216), an ATM inhibitor of formula (I) is prepared. Specifically, 7-fluoro-1-isopropyl-3-methyl-8-[6-[3-(1-piperidyl)propoxy]-3-pyridyl]imidazo[4,5-c]quinolin-2-one:

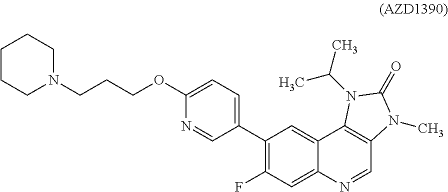 [SMILES: CC(C)n1c(=O)n(C)c2cnc3cc(F)c(-c4ccc(OCCCN5CCCCC5)nc4)cc3c21]

can be prepared according to Example 2 of WO2017/046216.

### Example 3: Antitumor Test (1)

Combination of antibody-drug conjugate DS-8201 (trastuzumab deruxtecan) with ATM inhibitor AZD1390 (7-fluoro-1-isopropyl-3-methyl-8-[6-[3-(1-piperidyl)propoxy]-3-pyridyl]imidazo[4,5-c]quinolin-2-one)

Method:

A high-throughput combination screen was run, in which three breast cancer cell lines with diverse HER2 expression and one gastric cell line with high HER2 expression (Table 1) were treated with combinations of DS-8201 and AZD1390 (ATM inhibitor).

TABLE 1

| Cell line | HER2 expression | Cancer type |
|---|---|---|
| KPL-4 | High | Breast (HER2+) |
| NCI-N87 | High | Gastric |
| MDA-MB-468 | Low | Breast (TNB) |
| T47D | Low | Breast (ER+) |

The readout of the screen was a 7-day cell titer-glo cell viability assay, conducted as a 6×6 dose response matrix for each combination (5-point log serial dilution for DS-8201, and half log serial dilution for partners). In addition, trastuzumab and exatecan (DNA topoisomerase I inhibitor) were also screened in parallel with AZD1390. Combination activity was assessed based on a combination of the ΔEmax and HSA synergy scores.

Results:

Results are shown in FIGS. 12A and 12B, and Table 2.

FIG. 12A shows matrices of measured cell viability signals. X axes represent drug A (DS-8201), and Y axes represent drug B (AZD1390). Values in the box represent the ratio of cells treated with drug A+B compared to DMSO control at day 7. All values are normalised to cell viability values at day 0. Values between 0 and 100 represent % growth inhibition and values above 100 represent cell death.

FIG. 12B shows HSA excess matrices. Values in the box represent excess values calculated by the HSA (Highest Single Agent) model.

Table 2 below shows HSA synergy and Loewe additivity scores:

TABLE 2

| Cell line | KPL4 | NCI-N87 | MDA-MB-468 | T47D |
|---|---|---|---|---|
| HSA synergy score | 47.4 | 50.0 | 11.5 | 4.0 |
| Loewe synergy score | 47.4 | 50.0 | 11.5 | 4.0 |

Loewe Dose Additivity predicts the expected response if the two compounds act on the same molecular target by means of the same mechanism. It calculates additivity based on the assumption of zero interaction between the compounds and it is independent from the nature of the dose-response relationship.
HSA (Highest Single Agent) [Berenbaum 1989] quantifies the higher of the two single compound effects at their corresponding concentrations. The combined effect is compared with the effect of each single agent at the concentration used in the combination. Excess over the highest single agent effect indicates cooperativity. HSA does not require the compounds to affect the same target.
Excess Matrix: For each well in the concentration matrix, the measured or fitted values are compared to the predicted non-synergistic values for each concentration pair. The predicted values are determined by the chosen model. Differences between the predicted and observed values may indicate synergy or antagonism, and are shown in the Excess Matrix. Excess Matrix values are summarized by the combination scores Excess Volume and Synergy Score.

As seen from FIGS. 12A and 12B, and Table 2, AZD1390 (AZ13791971) in combination with DS-8201 showed synergistic activity and increased cell death in HER2+ cell lines KPL4 and NCI-N87 at EMAX (0.3 μM AZD1390 and 10 μg/ml (0.064 μM) DS-8201). Combination activity was also observed at lower concentrations. Single agent AZD1390 was not active within the selected concentration range. In addition, AZD1390 and DS-8201 showed combination activity at DS-8201 Emax in HER2 low cell lines MDA-MB-468 and T47D.

The results demonstrate that ATM inhibition using AZD1390 enhances the antitumor efficacy of DS-8201 in both high and low HER2-expressing cell lines in vitro. AZD1390 showed synergistic combination activity and increased cell death in HER2 high cell lines. Beneficial combination activity was also observed in HER2 low cancer cell lines.

### Example 4: Antitumor Test (2)

Combination of antibody-drug conjugate DS-8201 (trastuzumab deruxtecan) with ATM inhibitor AZD1390 (7-fluoro-1-isopropyl-3-methyl-8-[6-[3-(1-piperidyl)propoxy]-3-pyridyl]imidazo[4,5-c]guinolin-2-one)

DS-8201 or exatecan mesylate was tested alone and in combination with AZD1390 in cancer cell lines with varying HER2 expression levels.

Method:

Cells grown in their respective conditions were plated in 96-well plates at optimal density to allow linear proliferation for the duration of the assay (4 to 8 days; duration of treatment is dependent on the growth rate of each cell line). Immediately after plating, the cells were dosed with the indicated compounds for a total volume of 200 μL/well and placed in the incubator. Combinations were conducted as a 6×8 concentration response matrix for each combination. At the endpoint, the cells were fixed in 2% PFA for 20 minutes at room temperature. In order to obtain the number of cells at the start of treatment, one additional plate was used for each experiment and fixed after cells attached. The cells were then permeabilised in 0.5% Triton-X100 in PBS for 10 minutes. After a PBS wash, the cells were blocked in 5% FBS in PBS 1 h at RT and incubated with primary antibodies in 5% FBS+0.05% triton overnight at 4° C. After 3 washes in PBS cells were incubated with secondary antibodies in 5% FBS+0.05% triton with Hoechst33258 for 1 h at room temperature. After 3 washes in PBS, the cells were scanned with a Cellinsight instrument with a 10×objective and 9 fields/well. Images were analysed using Columbus for cell count based on nuclear Hoechst staining and nuclear intensity of other biomarkers investigated. The total cell count/well was used to calculate the relative growth in each well compared to solvent control. To calculate the synergy scores, the growth inhibition data were analysed using Combenefit software (Di Veroli G Y et al., Bioinformatics 2016, 32(18), 2866-8). The mean/well of the sum of the nuclear intensity of the IF biomarkers was also expressed relative to solvent control.

Results:

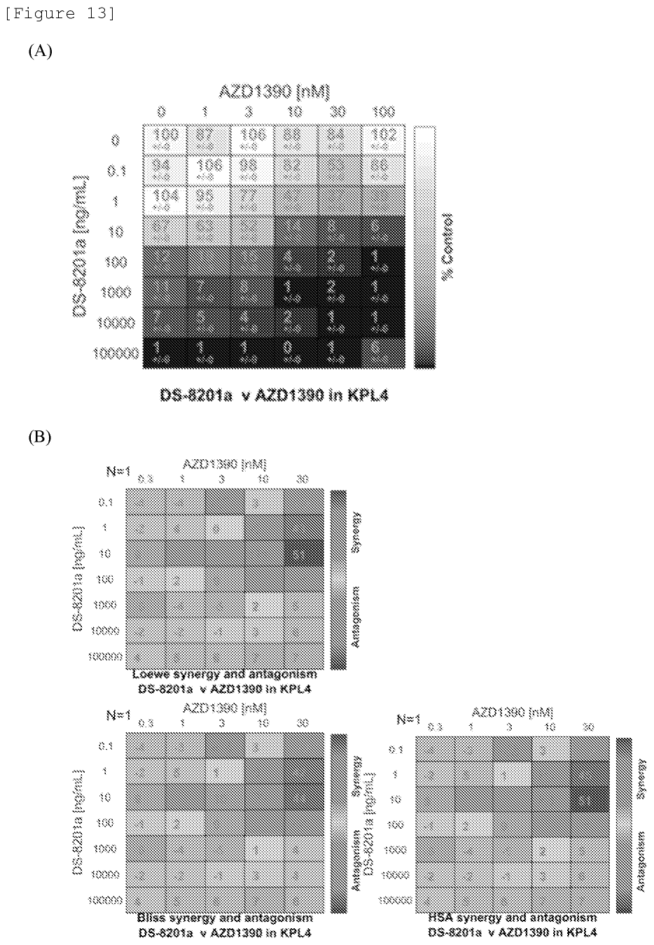

Results are shown in FIGS. 13 to 15 and Tables 3 and 4.

Table 3 below shows the monotherapy activities of DS-8201, exatecan and AZD1390 for cell lines used in the in vitro studies:

TABLE 3

| | | | | Exatecan | |
|---|---|---|---|---|---|
| | Tumour | HER2 | DS-8201 GI₅₀ | mesylate GI₅₀ | AZD1390 GI₅₀ |
| Cell line | type | expression | (ng/ml) | (nM) | (nM) |
| NCI-N87 | Gastric | High | 19 | 0.503 | >100 |
| KPL4 | Breast | High | 44 | 1.181 | >100 |
| MDA-MB-468 | Breast | Low | 3367 | 0.162 | >100 |
| SK-OV-3 | Ovarian | Moderate | 4674 | 0.914 | >100 |
| JIMT-1 | Breast | Expressed | 22609 | 0.745 | >100 |
| DU145 | Prostate | Low | 18361 | 0.564 | Nd |
| DU145-SLFN11 KO | Prostate | Low | 17080 | 0.450 | Nd |
| (CRISPR-Cas9 gene | | | | | |
| knockout of SLFN11) | | | | | |

*Nd: not determined

FIG. 13 shows synergy matrices for combinations with DS-8201 and AZD1390 in HER2-high KPL4 cell line.

In FIG. 13, (A) shows the relative total cell (nuclear) counts as percentage of DMSO vehicle control (control=100%, no cells remaining=0%; dark areas are regions with very low total cell count), and (B) shows calculated synergy matrices for Loewe, Bliss and HSA scores (higher =more synergy; dark areas are regions with high combination synergy).

Table 4 below shows the overall sum of synergy scores (Loewe, Bliss and HSA) for DS-8201 in combination with AZD1390:

TABLE 4

| | DS-8201 + | DS-8201 + | DS-8201 + |
|---|---|---|---|
| | AZD1390 | AZD1390 | AZD1390 |
| Cell line | Loewe | Bliss | HSA |
| KPL4 | 93.97 | 88.33 | 96.93 |
| MDA-MB-468 | 39.21 | 39.17 | 41.03 |
| SK-OV-3 | 56.27 | 57.07 | 64.79 |
| JIMT-1 | 44.68 | 33.67 | 44.72 |

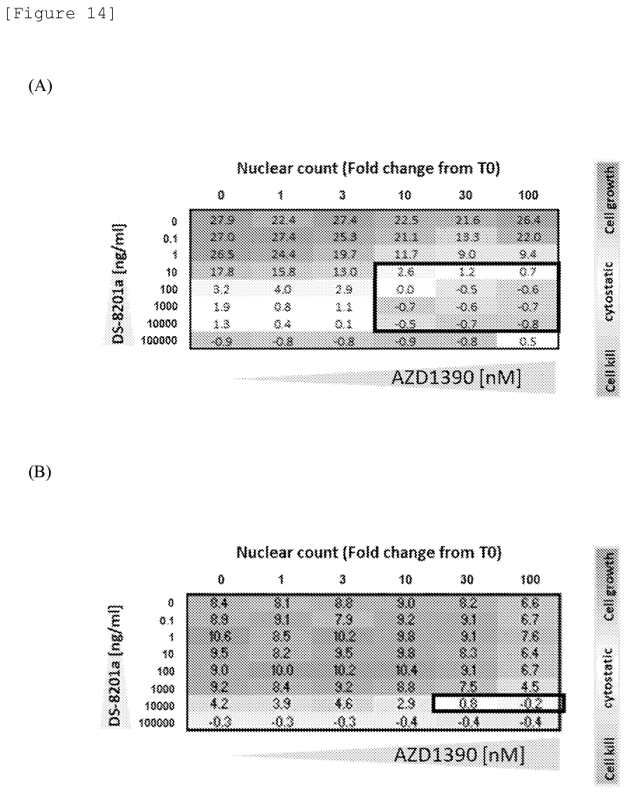

FIG. 14 shows fold change in total cells remaining after 4-8 days treatment compared to time zero for combinations of DS-8201 with AZD1390 in (A) HER2-high KPL4 cell line and (B) HER2-negative MDA-MB-468 cell line. Positive values indicate growth (fold increase), zero value indicates cytostasis and negative values represent net cell loss and surrogate for cell death. Boxed areas show regions of cytostasis or cell loss of combination compared to monotherapies.

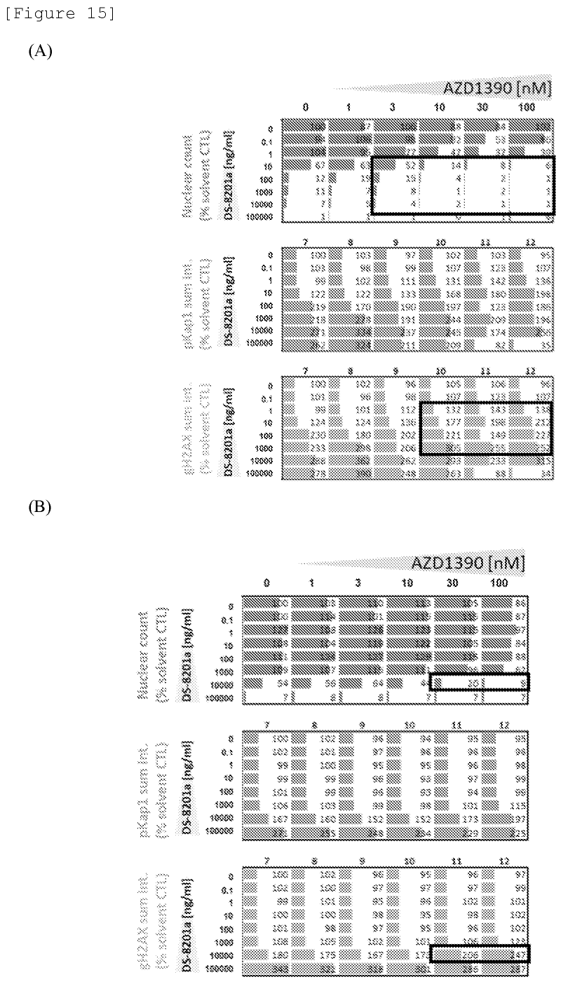

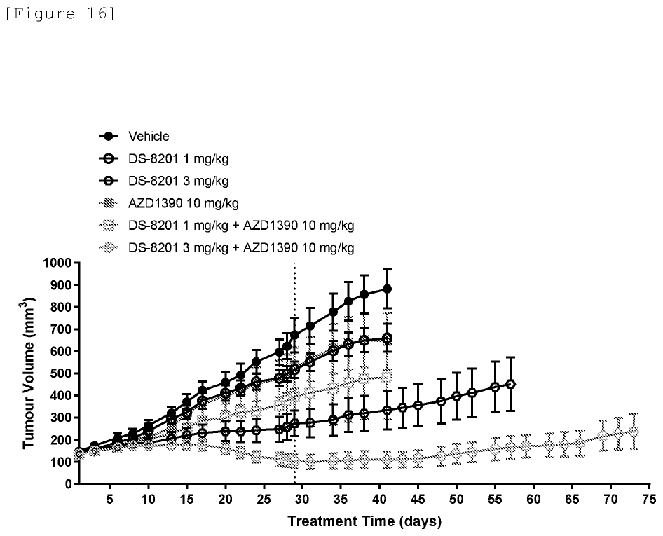

FIG. 15 shows induction of ATM-dependent KAP1 pSer824 signalling, DNA double strand break damage (yH2AX) biomarkers or percentage of cell number (vs solvent control) for combinations of DS-8201 with AZD1390 in (A) HER2-high KPL4 cell line or (B) HER2-low MDA-MB-468 cell line. Boxed areas show regions of increased induction of DNA damage response, DNA damage or cell loss of combination compared to monotherapies.

According to the results above, in the high-HER2 KPL4 breast cancer cell line models, synergistic activity and cell death were observed at clinically relevant concentrations of DS-8201 (and exatecan) in combination with ATM inhibitor AZD1390. In addition DS-8201 (and exatecan) induced in a concentration dependent manner biomarkers of ATM (KAP1 pSer824) activation and DNA strand breaks (yH2AX), which was further augmented in combination with AZD1390. In the HER2-negative MDA-MB-468 breast cancer cell line, weak combination activity and poor DNA damage response pathway activation were observed in combination DS-8201, while exatecan still showed combination activity, supporting the HER2 and tumor targeting dependency of DS-8201 but not with free exatecan. These data show strong potentiation of the activity with DS-8201 when combined with ATM inhibitor AZD1390 which is dependent on tumor HER2 expression and therefore may offer an increased therapeutic index compared to free topoisomerase-I inhibitors.

### Example 5: Antitumor Test (3)—In Vivo

Combination of antibody-drug conjugate DS-8201 (trastuzumab deruxtecan) with ATM inhibitor AZD1390 (7-fluoro-1-isopropyl-3-methyl-8-[6-[3-(1-piperidyl)propoxy]-3-pyridyl]imidazo[4,5-c]quinolin-2-one)

Method:

Female Nude mice (Charles River) aged 5-8 weeks were used, following 7 days acclimatisation before entry into the study. 1×10⁷ NCI-N87 tumour cells (1:1 in Matrigel) were implanted subcutaneously onto the flank of the female Nude mice. When tumours reached approximately 150 mm³, similar-sized tumours were randomly assigned to treatment groups as shown in Table 5:

TABLE 5

| | | Route of | Dosing Schedule |
|---|---|---|---|
| Treatment | Dose | administration | (28 days) |
| Vehicle | — | IV + PO | Single dose + BID |
| DS8201 | 3 mg/kg | IV | Single dose |
| DS8201 | 1 mg/kg | IV | Single dose |
| AZD1390 | 10 mg/kg | PO | QD × 28 |
| DS8201 + | 1 mg/kg or | IV + PO | Single dose + |
| AZD1390 | 3mg/kg + 10 mg/kg | | QD × 27 (24 h delay) |

The dose of compound for each animal was calculated based on the individual body weight on the day of dosing. AZD1390 was administered 24 hours post the DS-8201 dose. Duration of dosing was for 28 days (1 cycle) unless otherwise stated.

Formulation of DS-8201 at 3 mg/kg and 1 mg/kg

The dosing solutions for DS-8201 were prepared on the day of dosing by diluting the DS-8201 stock (20.1 mg/ml) in 25 mM histidine buffer, 9% sucrose (pH5.5) to 0.6 mg/ml, and 0.2 mg/ml for the 3 mg/kg and 1 mg/kg dosing solutions, respectively. Each dosing solution was mixed well using a pipette before administration via IV injection at a dosing volume of 5 ml/kg.

Formulation of AZD1390 at 10 mg/kg

To formulate for a 10 mg/kg dosing solution, a final concentration of 1 mg/ml AZD1390 was prepared which resulted in a dosing volume of 10 ml/kg for PO dosing. Initially a 5 mg/ml dosing solution was prepared in 0.5% HPMC containing 0.1% Tween 80 (vehicle). The compound was left stirring overnight before further dilution to a 1 mg/ml dosing solution on the day of dosing. The dosing solution was protected from light and kept at room temperature for up to 4 days being continually mixed. The final dosing matrix for 10 mg/kg AZD1390 was a white suspension.

Measurements

Tumour growth inhibition (TGI) from the start of the study to the day of tumour measurements was assessed by comparison of the geometric mean change in tumour volume for the control and treated groups. Tumour regressions were calculated as the percentage reduction in tumour volume from baseline (pre-treatment) value:

% Regression=(1−RTV)*100%,

where RTV=Geometric Mean Relative Tumour Volume. Statistical significance was evaluated using one-tailed t-test of (log(relative tumour volume)=log(final vol/start vol)) at the day of final measure, comparing to vehicle control.

Results:

Tumour volumes for treatments with DS-8201 and/or AZD1390 are shown in FIG. 16. Data represents change in tumour volume over time for treatment groups. The dotted line in FIG. 16 represents end of dosing periods. For full dose and schedule information, refer to Table 5 above. Values shown are mean±SEM; n=10 initially for vehicle-treated mice and n=8 for all other treatment groups.

TGI best responses (maximum TGI/regression) following treatment with DS-8201 or AZD1390 alone or with DS-8201 in combination with AZD1390, in NCI-N87 xenograft, are shown in Table 6:

TABLE 6

| | | Best | | |
|---|---|---|---|---|
| | | response | | |
| Treatment | Best response | Days post | p-value vs | |
| group | % TGI/regression | treatment | vehicle | Significance |
| DS-8201 | 84 | 33 | 0.00071 | *** |
| 3 mg/kg | | | | |
| DS-8201 | 22 | 37 | 0.025 | * |
| 1 mg/kg | | | | |
| AZD1390 | 37 | 40 | 0.024 | * |
| 10 mg/kg | | | | |
| DS-8201 | 82 | 40 | 0.019 | * |
| 1 mg/kg + | | | | |
| AZD1390 | | | | |
| 10 mg/kg | | | | |
| DS-8201 | 143 | 33 | <0.0001 | *** |
| 3 mg/kg + | (regression) | | | |
| AZD1390 | | | | |
| 10 mg/kg | | | | |

Monotherapy with DS-8201 at 3 mg/kg showed maximum tumour growth inhibition (TGI) of 84% at day 33 post treatment. At 1 mg/kg DS-8201 showed a maximum TGI of 22% at day 37 post treatment. AZD1390 monotherapy achieved a maximum TGI of 37% at day 40 post treatment. Combination treatment with DS-8201 at 1 mg/kg resulted in a significant reduction in NCI-N87 tumour burden compared to vehicle-treated control mice, with significant effect being observed by DS-8201 1 mg/kg+AZD1390 with a maximum TGI at 82% 40 days post treatment.

Combination therapy using higher DS-8201 3 mg/kg dose with AZD1390 achieved tumour regressions with a maximum TGI of 143% at day 33 post treatment and showed better response than either respective monotherapies.

All treatment groups were tolerated and no consistent differences in average bodyweights were observed between vehicle, monotherapy or combination groups.

### Example 6: Inhibition of ATM Signaling

Combination of DS-8201 with ATM inhibitor AZD1390

Method

Gastric cancer NCI-N87 and breast carcinoma KPL4 cell lines were cultured in RPMI 1640 supplemented with 10% FCS in a humidified incubator at 37° C. with 5% CO2. Cells were plated in 6-well plates at optimal density to allow linear proliferation for the duration of the assay. Two days after plating, cells were dosed with the indicated compounds (AZD1390 alone, or combined with DS-8201 or exatecan mesylate) and placed back in the incubator. 7 h, 24 h or 48 h after dosing, whole-cell extracts were obtained by lysis in 50 mM Tris-HCl pH 7.5, 2% SDS containing protease and phosphatase inhibitors. Lysates were boiled for 5 minutes at 95° C. Protein concentration was measured using a Nanodrop at 240 nm and 50 μg of lysate were loaded in 4-12% Bis Tris gels. Proteins were transferred using iblot2. Primary antibodies (see Table 7) were incubated overnight at 4° C. in 3% milk TBS-tween 0.05% and HRP-conjugated secondary antibodies for 1 h at room temperature. Blots were imaged using a G-box.

TABLE 7

| | Host | Catalog | |
|---|---|---|---|
| Antibody target name | species | number | Supplier |
| ATR (P-Thr1989) | rabbit | GTX128145 | GeneTex |
| CHK1 (P-Ser345) (clone 133D3) | rabbit | 2348 | Cell signalling (NEB) |
| KAP1 (P-Ser824) | rabbit | ab70369 | Abcam |
| Rad50 (P-Ser635) | rabbit | 14223 | Cell signalling (NEB) |
| RPA 32 (P-Ser4/Ser8) | rabbit | A300-245A | Bethyl Laboratories |
| γH2Aχ (P-Ser139) (clone JBW301) | mouse | 05-636 | Merck Millipore |
| CHK2 (P-Thr68) | rabbit | 2661 | Cell signalling (NEB) |
| ATM phosphoSer 1981 monoclonal | mouse | MAB3806 | Millipore |
| p53 (P-Ser15) (clone 16G8) | mouse | 9286 | Cell signalling (NEB) |
| CDC2 (P-Tyr15) | rabbit | 4539 | Cell signalling (NEB) |

Results:

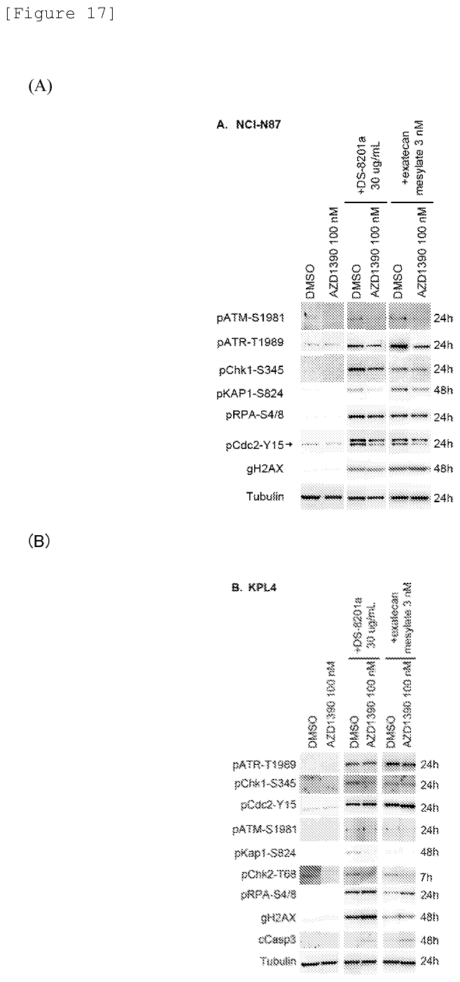

Results are shown in FIG. 17, in the form of antibody blot images obtained using AZD1390 alone, or combined with DS-8201 (or exatecan mesylate), in (A) NCI-N87 (gastric cancer) and (B) KPL4 (breast carcinoma) cell lines.

In both Her2-high NCI-N87 and KPL4, exposure to DS-8201 at 30 μg/mL or the warhead (exatecan mesylate) induced activation of the ATM pathway, as shown by increase in pATM-S1981, pChk2-T68, and pKap1-S824. Combination with AZD1390 at 100 nM inhibited the activation of pATM, pChk2, and pKAP1, while exacerbating the DNA damage (pRPA, gH2AX), ultimately leading to increased cell death (cCasp3).

Thus, it is shown that AZD1390 inhibits DS-8201-induced ATM signaling.

### Example 7: Antitumor Test (4)

Combination of antibody-drug conjugate DS-8201 (trastuzumab deruxtecan) with ATM inhibitor AZD1390 (7-fluoro-1-isopropyl-3-methyl-8-[6-[3-(1-piperidyl)propoxy]-3-pyridyl]imidazo[4,5-c]guinolin-2-one)

Method:

A high-throughput combination screen was run, in which a lung cancer cell line with high HER2 expression (Table 8) was treated with combinations of DS-8201 and AZD1390.

TABLE 8

| Cell line | HER2 expression | Cancer type |
|---|---|---|
| NCI-H2170 | High | NSCLC squamous |

The readout of the screen was a 7-day cell titer-glo cell viability assay, conducted as a 6×6 dose response matrix for each combination (both DS-8201 and AZD1390 were used at half-log serial dilutions).

Combination activity was assessed based on a combination of the ΔEmax and HSA synergy scores.

Results:

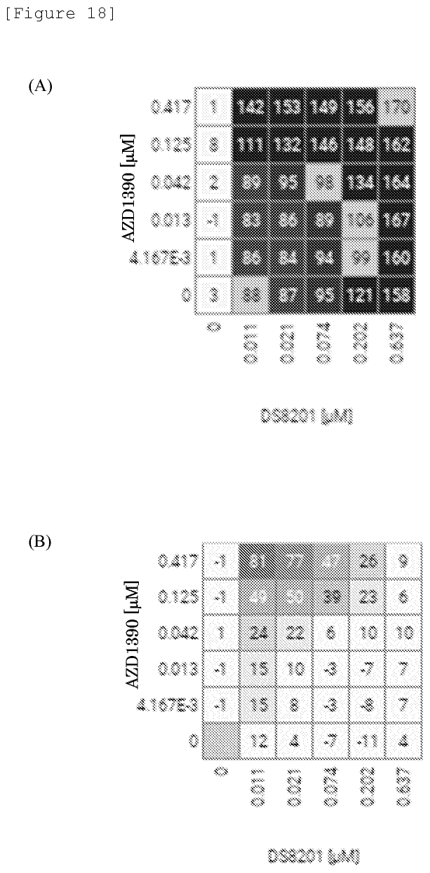

Results are shown in FIGS. 18A and 18B and Table 9.

FIG. 18A shows a matrix of measured cell viability signals. X axis represents drug A (DS-8201), and Y axis represents drug B (AZD1390). Values in the box represent the ratio of cells treated with drug A+B compared to DMSO control at day 7. All values are normalised to cell viability values at day 0. Values between 0 and 100 represent % growth inhibition and values above 100 represent cell death.

FIG. 18B shows an HSA excess matrix. Values in the box represent excess values calculated by the HSA (Highest Single Agent) model.

Table 9 below shows HSA additivity and Loewe synergy scores:

TABLE 9

| Cell line | NCI-H2170 |
|---|---|
| HSA synergy score | 7.68 |
| Loewe synergy score | 7.68 |

As seen from FIGS. 18A and 18B, and Table 9, AZD1390 interacted synergistically with DS-8201 and also increased cell death in a HER2 high lung cell line.

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiments may be practiced in many ways and the claims include any equivalents thereof.

Free Text of Sequence Listing
SEQ ID NO: 1—Amino acid sequence of a heavy chain of an anti-HER2 antibody
SEQ ID NO: 2—Amino acid sequence of a light chain of an anti-HER2 antibody
SEQ ID NO: 3—Amino acid sequence of a heavy chain CDRH1 [=amino acid residues 26 to 33 of SEQ ID NO: 1]
SEQ ID NO: 4—Amino acid sequence of a heavy chain CDRH2 [=amino acid residues 51 to 58 of SEQ ID NO: 1]
SEQ ID NO: 5—Amino acid sequence of a heavy chain CDRH3 [=amino acid residues 97 to 109 of SEQ ID NO: 1]
SEQ ID NO: 6—Amino acid sequence of a light chain CDRL1 [=amino acid residues 27 to 32 of SEQ ID NO: 2]
SEQ ID NO: 7—Amino acid sequence comprising an amino acid sequence of a light chain CDRL2 (SAS) [=amino acid residues 50 to 56 of SEQ ID NO: 2]
SEQ ID NO: 8—Amino acid sequence of a light chain CDRL3 [=amino acid residues 89 to 97 of SEQ ID NO: 2]
SEQ ID NO: 9—Amino acid sequence of a heavy chain variable region [=amino acid residues 1 to 120 of SEQ ID NO: 1]
SEQ ID NO: 10—Amino acid sequence of a light chain variable region [=amino acid residues 1 to 107 of SEQ ID NO: 2]
SEQ ID NO: 11—Amino acid sequence of a heavy chain [=amino acid residues 1 to 449 of SEQ ID NO: 1]

1. A pharmaceutical product comprising an anti-HER2 antibody-drug conjugate and an ATM inhibitor for administration in combination, wherein the anti-HER2 antibody-drug conjugate is an antibody-drug conjugate in which a drug-linker represented by the following formula: 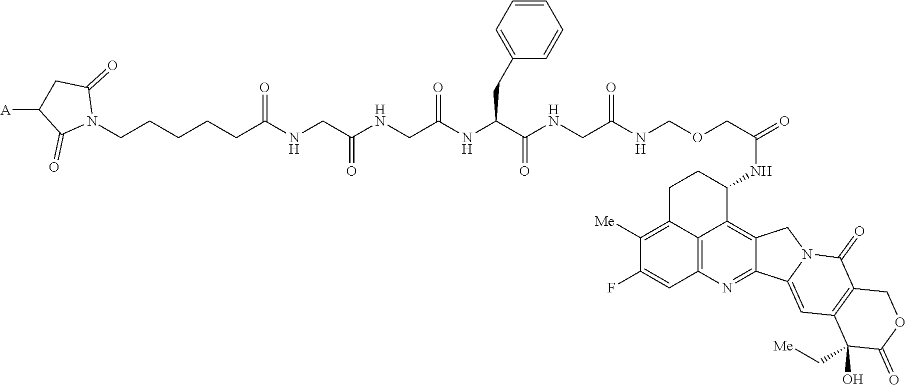
wherein A represents the connecting position to an antibody, is conjugated to an anti-HER2 antibody via a thioether bond.

2. The pharmaceutical product according to claim 1, wherein the ATM inhibitor is a compound represented by the following formula (I): 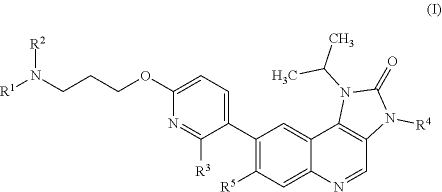
wherein:
R¹ is methyl;
R² is hydrogen or methyl; or R¹ and R² together with the nitrogen atom to which they are bonded form an azetidinyl, pyrrolidinyl or piperidinyl ring;
R³ is hydrogen or fluoro;
R⁴ is hydrogen or methyl; and
R⁵ is hydrogen or fluoro,
or a pharmaceutically acceptable salt thereof.

3. The pharmaceutical product according to claim 2 wherein, in formula (I), R¹ and R² are both methyl; or R¹ and R² together with the nitrogen atom to which they are bonded form an azetidinyl, pyrrolidinyl or piperidinyl ring.

4. The pharmaceutical product according to claim 2 or claim 3 wherein, in formula (I), R¹ and R² together with the nitrogen atom to which they are bonded form an azetidinyl, pyrrolidinyl or piperidinyl ring.

5. The pharmaceutical product according to any one of claims 2 to 4 wherein, in formula (I), R³ is hydrogen.

6. The pharmaceutical product according to any one of claims 2 to 5 wherein, in formula (I), R⁴ is methyl.

7. The pharmaceutical product according to any one of claims 2 to 6 wherein, in formula (I), R⁵ is fluoro.

8. The pharmaceutical product according to claim 2 wherein, in formula (I):
R¹ is methyl;
R² is methyl; or R¹ and R² together with the nitrogen atom to which they are bonded form an azetidinyl, pyrrolidinyl or piperidinyl ring;
R³ is hydrogen or fluoro;
R⁴ is methyl; and
R⁵ is hydrogen or fluoro.

9. The pharmaceutical product according to claim 2, wherein the ATM inhibitor is 7-fluoro-1-isopropyl-3-methyl-8-[6-[3-(1-piperidyl)propoxy]-3-pyridyl]imidazo[4,5-c]quinolin-2-one (AZD1390) represented by the following formula: 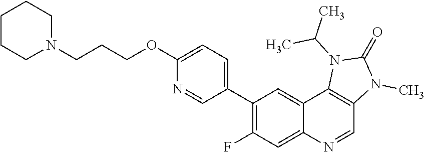 [SMILES: CC(C)n1c(=O)n(C)c2cnc3cc(F)c(-c4ccc(OCCCN5CCCCC5)nc4)cc3c21]
or a pharmaceutically acceptable salt thereof.

10. The pharmaceutical product according to any one of claims 1 to 9, wherein the anti-HER2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence consisting of amino acid residues 1 to 3 of SEQ ID NO: 7 and CDRL3 consisting of an amino acid sequence represented by SEQ ID NO: 8.

11. The pharmaceutical product according to any one of claims 1 to 9, wherein the anti-HER2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10.

12. The pharmaceutical product according to any one of claims 1 to 9, wherein the anti-HER2 antibody is an antibody comprising a heavy chain consisting of an amino acid sequence represented by SEQ ID NO: 1 and a light chain consisting of an amino acid sequence represented by SEQ ID NO: 2.

13. The pharmaceutical product according to any one of claims 1 to 9, wherein the anti-HER2 antibody is an antibody comprising a heavy chain consisting of an amino acid sequence represented by SEQ ID NO: 11 and a light chain consisting of an amino acid sequence represented by SEQ ID NO: 2.

14. The pharmaceutical product according to any one of claims 1 to 13, wherein the anti-HER2 antibody-drug conjugate is represented by the following formula: 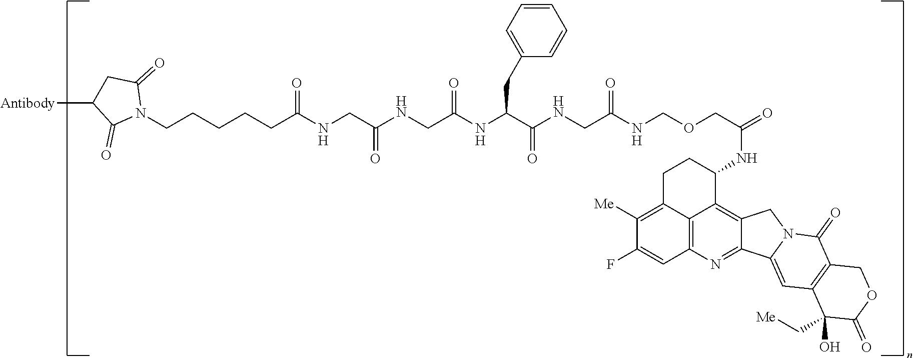
wherein ‘Antibody’ indicates the anti-HER2 antibody conjugated to the drug-linker via a thioether bond, and n indicates an average number of units of the drug-linker conjugated per antibody molecule in the antibody-drug conjugate, wherein n is in the range of from 7 to 8.

15. The pharmaceutical product according to any one of claims 1 to 14, wherein the anti-HER2 antibody-drug conjugate is trastuzumab deruxtecan (DS-8201).

16. The pharmaceutical product according to any one of claims 1 to 15, wherein the product is a composition comprising the anti-HER2 antibody-drug conjugate and the ATM inhibitor, for simultaneous administration.

17. The pharmaceutical product according to any one of claims 1 to 15, wherein the product is a combined preparation comprising the anti-HER2 antibody-drug conjugate and the ATM inhibitor, for sequential or simultaneous administration.

18. The pharmaceutical product according to any one of claims 1 to 17, wherein the product is for treating cancer.

19. The pharmaceutical product according to claim 18, wherein the cancer is at least one selected from the group consisting of breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioma, glioblastoma multiforme, osteosarcoma, sarcoma, and melanoma.

20. The pharmaceutical product according to claim 18, wherein the cancer is breast cancer.

21. The pharmaceutical product according to claim 20, wherein the breast cancer has a HER2 status score of IHC 3+.

22. The pharmaceutical product according to claim 20, wherein the breast cancer is HER2 low-expressing breast cancer.

23. The pharmaceutical product according to claim 20, wherein the breast cancer has a HER2 status score of IHC 2+.

24. The pharmaceutical product according to claim 20, wherein the breast cancer has a HER2 status score of IHC 1+.

25. The pharmaceutical product according to claim 20, wherein the breast cancer has a HER2 status score of IHC >0 and <1+.

26. The pharmaceutical product according to claim 20, wherein the breast cancer is triple-negative breast cancer.

27. The pharmaceutical product according to claim 18, wherein the cancer is gastric cancer.

28. The pharmaceutical product according to claim 18, wherein the cancer is colorectal cancer.

29. The pharmaceutical product according to claim 18, wherein the cancer is lung cancer.

30. The pharmaceutical product according to claim 29, wherein the lung cancer is non-small cell lung cancer.

31. The pharmaceutical product according to claim 18, wherein the cancer is pancreatic cancer.

32. The pharmaceutical product according to claim 18, wherein the cancer is ovarian cancer.

33. The pharmaceutical product according to claim 18, wherein the cancer is prostate cancer.

34. The pharmaceutical product according to claim 18, wherein the cancer is kidney cancer.

35. A pharmaceutical product as defined in any one of claims 1 to 17, for use in treating cancer.

36. The pharmaceutical product for the use according to claim 35, wherein the cancer is at least one selected from the group consisting of breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioma, glioblastoma multiforme, osteosarcoma, sarcoma, and melanoma.

37. The pharmaceutical product for the use according to claim 35, wherein the cancer is breast cancer.

38. The pharmaceutical product for the use according to claim 37, wherein the breast cancer has a HER2 status score of IHC 3+.

39. The pharmaceutical product for the use according to claim 37, wherein the breast cancer is HER2 low-expressing breast cancer.

40. The pharmaceutical product for the use according to claim 37, wherein the breast cancer has a HER2 status score of IHC 2+.

41. The pharmaceutical product for the use according to claim 37, wherein the breast cancer has a HER2 status score of IHC 1+.

42. The pharmaceutical product for the use according to claim 37, wherein the breast cancer has a HER2 status score of IHC >0 and <1+.

43. The pharmaceutical product for the use according to claim 37, wherein the breast cancer is triple-negative breast cancer.

44. The pharmaceutical product for the use according to claim 35, wherein the cancer is gastric cancer.

45. The pharmaceutical product for the use according to claim 35, wherein the cancer is colorectal cancer.

46. The pharmaceutical product for the use according to claim 35, wherein the cancer is lung cancer.

47. The pharmaceutical product for the use according to claim 46, wherein the lung cancer is non-small cell lung cancer.

48. The pharmaceutical product for the use according to claim 35, wherein the cancer is pancreatic cancer.

49. The pharmaceutical product for the use according to claim 35, wherein the cancer is ovarian cancer.

50. The pharmaceutical product for the use according to claim 35, wherein the cancer is prostate cancer.

51. The pharmaceutical product for the use according to claim 35, wherein the cancer is kidney cancer.

52. Use of an anti-HER2 antibody-drug conjugate or an ATM inhibitor in the manufacture of a medicament for administration of the anti-HER2 antibody-drug conjugate and the ATM inhibitor in combination, wherein the anti-HER2 antibody-drug conjugate and the ATM inhibitor are as defined in any one of claims 1 to 15, for treating cancer.

53. The use according to claim 52, wherein the cancer is at least one selected from the group consisting of breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioma, glioblastoma multiforme, osteosarcoma, sarcoma, and melanoma.

54. The use according to claim 52, wherein the cancer is breast cancer.

55. The use according to claim 54, wherein the breast cancer has a HER2 status score of IHC 3+.

56. The use according to claim 54, wherein the breast cancer is HER2 low-expressing breast cancer.

57. The use according to claim 54, wherein the breast cancer has a HER2 status score of IHC 2+.

58. The use according to claim 54, wherein the breast cancer has a HER2 status score of IHC 1+.

59. The use according to claim 54, wherein the breast cancer has a HER2 status score of IHC >0 and <1+.

60. The use according to claim 54, wherein the breast cancer is triple-negative breast cancer.

61. The use according to claim 52, wherein the cancer is gastric cancer.

62. The use according to claim 52, wherein the cancer is colorectal cancer.

63. The use according to claim 52, wherein the cancer is lung cancer.

64. The use according to claim 63, wherein the lung cancer is non-small cell lung cancer.

65. The use according to claim 52, wherein the cancer is pancreatic cancer.

66. The use according to claim 52, wherein the cancer is ovarian cancer.

67. The use according to claim 52, wherein the cancer is prostate cancer.

68. The use according to claim 52, wherein the cancer is kidney cancer.

69. The use according to any one of claims 52 to 68 wherein the medicament is a composition comprising the anti-HER2 antibody-drug conjugate and the ATM inhibitor, for simultaneous administration.

70. The use according to any one of claims 52 to 68 wherein the medicament is a combined preparation comprising the anti-HER2 antibody-drug conjugate and the ATM inhibitor, for sequential or simultaneous administration.

71. A method of treating cancer comprising administering an anti-HER2 antibody-drug conjugate and an ATM inhibitor as defined in any one of claims 1 to 15 in combination to a subject in need thereof.

72. The method according to claim 71, wherein the cancer is at least one selected from the group consisting of breast cancer, gastric cancer, colorectal cancer, lung cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioma, glioblastoma multiforme, osteosarcoma, sarcoma, and melanoma.

73. The method according to claim 71, wherein the cancer is breast cancer.

74. The method according to claim 73, wherein the breast cancer has a HER2 status score of IHC 3+.

75. The method according to claim 73, wherein the breast cancer is HER2 low-expressing breast cancer.

76. The method according to claim 73, wherein the breast cancer has a HER2 status score of IHC 2+.

77. The method according to claim 73, wherein the breast cancer has a HER2 status score of IHC 1+.

78. The method according to claim 73, werein the breast cancer has a HER2 status score of IHC >0 and <1+.

79. The method according to claim 73, wherein the breast cancer is triple-negative breast cancer.

80. The method according to claim 71, wherein the cancer is gastric cancer.

81. The method according to claim 71, wherein the cancer is colorectal cancer.

82. The method according to claim 71, wherein the cancer is lung cancer.

83. The method according to claim 82, wherein the lung cancer is non-small cell lung cancer.

84. The method according to claim 71, wherein the cancer is pancreatic cancer.

85. The method according to claim 71, wherein the cancer is ovarian cancer.

86. The method according to claim 71, wherein the cancer is prostate cancer.

87. The method according to claim 71, wherein the cancer is kidney cancer.

88. The method according to any one of claims 71 to 87, wherein the method comprises administering the anti-HER2 antibody-drug conjugate and the ATM inhibitor sequentially.

89. The method according to any one of claims 71 to 87, wherein the method comprises administering the anti-HER2 antibody-drug conjugate and the ATM inhibitor simultaneously.
